(19) United States
(12) Patent Application Publication
(10) Pub. No.: US 2003/0097691 A1
(43) Pub. Date: May 22, 2003

(54) Nucleic acid-based marker for tree phenotype prediction and method thereof

(76) Inventors: Simon Potter, New Westminster (CA); Paul A. Watson, Vancouver (CA)

Correspondence Address: OGILVY RENAULT, 1981 MCGILL COLLEGE AVENUE, SUITE 1600, MONTREAL, QC, H3A2Y3, CA

(21) Appl. No.: 09/995,813

(22) Filed: Nov. 29, 2001

Related U.S. Application Data
(63) Continuation-in-part of application No. 09/494,501, filed on Jan. 31, 2000.
(60) Provisional application No. 60/118,103, filed on Feb. 1, 1999.

(51) Int. Cl. C12Q 1/68; A01H 7/00

(52) U.S. Cl. 800/295; 435/6; 800/319

(57) ABSTRACT

The present invention relates to a method of identifying tree lineage capable of expressing desired biological and/or biochemical phenotypes and method of producing such trees. It also relates to a method of identifying a genetic marker associated with a genetic locus conferring at least one enhanced property. Also it relates to a stand of clonal enhanced property trees produced by the method of the present invention, the genome of the trees containing the same genetic marker associated with the enhanced property relative to a value characteristic of the average of the genus. It relates also to a method of producing a family of trees wherein at least about half exhibit at least of enhanced property. The present invention also relates to a genetic map of QTLs of trees associated with enhanced properties. The present invention further relates to a genetic marker of fiber length of trees.

This application is a continuation-in-part of the application Ser. No. 09/494,501 filed on Jan. 31, 2000.

### BACKGROUND OF THE INVENTION

1. Field of the Invention

This invention is in the fields of tree improvement, forestry and pulp and paper evaluation technology. This invention allows for an enhanced selection efficiency for given trees from both natural and plantation populations with specific fiber and wood quality properties for value-added pulp and paper product lines.

2. Description of Prior Art

The utilization of species of the Populus genus of forest trees, particularly aspen and cottonwoods, as the cornerstone for the development of short-rotation intensive culture (SRIC) sustainable plantation forestry in the northern hemisphere has been promoted for a number of reasons, including greenhouse gas amelioration and phytoremediation. The primary driving force behind the implementation of SRIC Populus plantations, however, is their potential to alleviate the shortfall in world fiber supplies projected for 2010.

This threat has provided an impetus for the examination of alternative fiber sources. Many non-wood sources have been characterized. “The morphology, ultrastructure and chemistry of wheat straw: a pulping perspective but the most logical and industrially expedient solution to the problem is likely to lie in fast-growing hardwood tree species. In the Southern hemisphere (and some parts of Europe), eucalyptus species are the hardwood of choice being prized for their growth rate, inherent adaptability and excellent papermaking properties.

In the Northern hemisphere members of the genus Populus (including poplars and aspen) represent a similar opportunity having high growth rates—up to 30 m³/ha/yr in cold climates—producing pulps of high natural brightness and a wide range of fiber, pulping and pulp properties.

Advantageously, poplars are unique in the additional potential they offer for genetic improvement of wood quality traits. Hybrid poplars are particularly well suited to genetic mapping studies as they are readily amenable to interspecies crosses, the progeny grow rapidly, and they have a relatively small genome. These advantages imply that the identification and manipulation of genetic control elements in poplars will be at least twice as easy as in rival fast-growing species such as eucalypts, and forty times easier than in radiata pine. Information generated by studies of this kind is extremely valuable for a number of reasons. Genetic control elements can be used to both rapidly and easily identify superior clonal material in natural populations and to screen such material for plantation establishment. Additionally, knowledge of the genetic structure of superior clones will open the door to transgenic manipulation to produce “ideal” trees (ideotypes) for specific end-product applications.

In a previous study on a genetically well-characterized three-generation family of hybrid poplars (Populus trichocarpaX Populus deltoides—Family 331) developed by the University of Washington, this potential was assessed and exploited [PD4145]. Quantitative trait loci (QTL—genomic regions containing genes involved in the control of continuously variable traits*) for wood and fiber quality traits were determined. As an extension of, and complement to, this application, additional phenotypic information has been gathered for the same family grown at three separate sites. In this case, the industrially relevant traits examined were: *[Regions of DNA which contain multiple genes affecting the same physical trait are known as quantitative trait loci (QTLs). These regions are detected using genetic marker technology and their presence or absence can be statistically correlated in tree populations with the magnitude of a particular physiological trait, such as fiber length. This statistical association is based on the technique of multiple simultaneous linear regressions of trait data with genetic marker presence/absence data using computer software. In this way, genetic maps can be “scanned” for groups of markers which correlate with the trait of interest—this group of markers is then classified as bounding a QTL partially controlling that trait (in other words, the markers are not the genes involved in the control of the trait, but those genes exist within the region of DNA bounded by the markers—this method is known as interval mapping). The degree of association between the markers and the trait can be used to estimate the “strength” of the QTL, i.e., the percentage of the trait variance which that particular QTL can account for].

fiber coarseness

microfibril angle

macerated fiber yield

kraft pulp yield

pulp properties including strength and air resistance

kraft pulping H-factor

specific refining energy

lignin content

wood extractive compounds content

calcium salt accumulation

The first four properties examined, fiber coarseness, microfibril angle, pulp yield and lignin content, are all critical pulp and papermaking parameters. The properties of a sheet of paper are dependent on the structural characteristics of the fibers which compose that sheet, the two most important characteristics being the length of the fibers and their coarseness (a weight to length measure). Length is required for strength properties, particularly so for hardwood species as longer-fiberd hardwood pulps can be used to reduce the expensive softwood component of certain papermaking furnishes. In softwoods, increasing fiber length can actually be problematic as excessively long fibers are prone to flocculation. Coarseness is often (but not always, c.f. red and sugar maple) a reasonable indicator of the thickness of the fiber cell wall. Wall thickness determines whether the fibers will collapse to readily form flat ribbons, giving paper sheets a smooth surface, or be less uncollapsible providing sheet bulk and absorbancy. Consequently, coarser, generally thicker-walled, fibers (e.g. Douglas fir) resist collapse and produce open, absorbent, bulky sheets with low burst/tensile strength and high tear strength.

The structural framework of the cell wall of fibers is primarily provided by cellulose microfibrils in the thickest S2 layer, cemeted together with lignin. The lignin binds the microfibrils and prevents their lateral buckling under load. The parameter microfibril angle indicates the angle to the longitudinal axis of the fiber at which the microfibrils are wound around the cell in a spiral formation. The smaller the angle, the steeper the spiral (in general, microfibril angle is at its highest near the pith, decreases through the juvenile wood core and then reaches a stable level in the mature wood). Microfibril angle has a major effect on the physical strength of directed axially along the microfibril. The steeper the angle, the stronger the fiber and the higher the tensile modulus. In this capacity therefore, microfibril angle is a critical strength parameter for both pulp and paper and solid wood applications of forest species.

Pulp yield is a measure of the amount of fiber recovered from an initial charge of wood. A great deal of chemical engineering effort is routinely expended to achieve process improvements in yield of the order of 0.5-1.0%. (e.g. polysulfide process). As wood quality databases become gradually more comprehensive, it is clear that both inter- and intra-species variability for this parameter can vastly outweigh such a change. Indeed, recent research has suggested that choosing one aspen (Populus tremuloides) clone over another of the same species for pulping can result in a yield improvement of 4-6% at a given kappa number. The efficiency of the pulping process, and a number of subsequent papermaking parameters, are critically dependent on the amount and chemical composition of the lignin polymer found in the wood. Normal softwood lignin is mainly composed of guaiacylpropane subunits which are difficult to remove via conventional processes. By contrast, hardwood lignin is composed of both guaiacyl- and syringylpropane units, in which the ratio of the two phenylpropanes varies between species.

If the genetic control of the lignin biosynthetic pathway can be determined, it may be possible to assess softwood populations for clones with hardwood-like lignin or to produce more syringyl residues in softwood lignin. Transgenic manipulation is also possible and, indeed, several research groups are already manipulating some of the control enzymes of the lignin biosynthetic pathway with varying results.

Specific extractives of wood are well known to cause adverse effects on various aspects of pulp and papermaking, specifically pitch deposition and effluent toxicity, particularly for mechanical pulping operations.

It has been estimated that pitch deposition problems (such as dispersed wood resin, metal soaps, wood resin component polymerization and surface active agent foaming) cost the Canadian industry several hundred million dollars annually. These extractive effects in open systems are already disproportionate to their concentration (extractives comprise ˜1-5% of the weight of wood) and it is anticipated that the problems will be exacerbated by progress towards mill system closure. For species used in mechanical pulping, such as aspen and related species, there are additional problems with pulp brightening caused by high extractives content.

A number of research groups have previously noted that certain poplar species have an inherent tendency to accumulate mineral deposits, particularly calcium salt crystals in their wood. Evidence described in these papers suggests that these crystals do not represent abnormalities but rather are consistently present in some Populus lineages (particularly the sections Aigeros and Tacamahaca). The crystals were found to accumulate in the stem, branches, roots and within vessels and fibers frequently occluding them completely. This paper reports the confirmation of these findings using the well-characterized hybrid poplar family and documents the effects of these crystals on the pulp properties of the hybrid family.

It would be highly desirable to be provided with a nucleic acid-based marker for tree phenotype prediction and method thereof.

### SUMMARY OF THE INVENTION

The aim of the present invention is to provide a method for identifying individual trees having a superior phenotype.

A number of previous studies [Bradshaw, H. D., Villar, M., Watson, B. D., Otto, K. G., and Stewart, S. “Molecular genetics of growth and development in Populus III. A genetic linkage map of a hybrid poplar composed of RFLP, STS and RAPD markers,” Theor. Appl. Genet. 89, 551-558 (1994)] have suggested that growth, adaptive and wood quality traits are not controlled by huge numbers of genes with small effects but that they are determined by a few genes with large effects whose influences are tempered by environmental blurring. The method described in this application demonstrates that this situation also holds for fiber and wood quality property determinants. For each trait examined in the application (except certain kraft and APRMP pulping properties), QTL have been found which contribute significantly to the phenotypic variance observed for that trait.

Of greatest significance are certain QTL which have proven to be coincident in their location on the genetic map. The QTL for tensile index and air resistance are in the same genetic location and critically they also overlap the QTL for fiber coarseness and microfibril angle. This adds compelling evidence to the hypothesis that there is a causal relationship between these fiber properties and certain pulping characteristics and that rapid assessment of the former may potentially be an indicator of the latter. Equally significantly, other QTL are not coincident for example, those detected for lignin content, H-factor and pulp yield. The fact that these properties do not appear to be controlled by the same set of genes emphasizes the complexity of the determination of H-factor and yield properties and further indicates that simple alteration of lignin content may not be the key to reliable manipulation of pulping characteristics of trees.

These QTL can be used for the development of marker-assisted breeding or rapid assessment techniques (based on assays or microarray technologies) which could save the pulp and paper industry much time and money in the refinement and development of new and better products based on purpose-grown fiber of known quality. The QTL can be applied to enhance and direct tree-breeding experiments to the improvement of wood quality traits and to the rapid assessment of natural stands on the basis of their fiber and wood quality properties.

In accordance with the present invention there is provided a method of identifying tree lineage capable of expressing desired biological and/or biochemical phenotypes comprising the steps of:

a) obtaining a nucleic acid sample from the trees of pure species and/or hybrids thereof;

b) obtaining either a restriction pattern (RFLP) or PCR-fingerprint by subjecting the nucleic acid of step (a) to at least one restriction enzyme and/or standard PCR conditions with at least one specific primer;

c) correlating the PCR-fingerprint or restriction pattern of step (b) to at least one selected biological and/or biochemical phenotype of the tree wherein the phenotype is associated with a genetic locus identified by and/or associated with the PCR fingerprint or restriction pattern.

The method in accordance with a preferred embodiment of the present invention, wherein the PCR-fingerprint is selected from the group consisting of RAPD, AFLP, CAP and SCAR.

The method in accordance with a preferred embodiment of the present invention, wherein the correlating of step (c) further comprises the sequencing of polymorphic DNA products associated with the genetic locus associated with the phenotype.

The method in accordance with a preferred embodiment of the present invention, wherein DNA sequences represent candidate genes or are highly linked to candidate genes for use as DNA markers as in step (c).

The method in accordance with a preferred embodiment of the present invention, wherein the DNA sequences are physically and/or genetically linked to candidate genes.

The method in accordance with a preferred embodiment of the present invention, wherein the tree of pure species and/or hybrid thereof is naturally or artificially produced.

The method in accordance with a preferred embodiment of the present invention, wherein the sample of step (a) is obtained from a leaf, cambium, root, bud, stem, cork, phloem, flower, seed, seeds pods or xylem.

The method in accordance with a preferred embodiment of the present invention, wherein the tree is of the genus selected from the group consisting of: Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

In accordance with the present invention, there is provided a method of identifying a genetic marker associated with a genetic locus conferring at least one enhanced property selected from the group consisting of fiber length, fiber coarseness, DBII (diameter at breast height), microfibril angle, density, pulp strength, pulp yield, lignin content, pitch propensity and calcium accumulation in a family of trees, which comprises the steps of:

a) obtaining a sexually mature parent tree exhibiting enhanced properties;

b) obtaining a plurality of progeny trees of the parent tree by performing self or cross-pollination;

c) assessing multiple progeny trees for each of a plurality of genetic markers;

d) identifying genetic markers segregating in an essentially Mendelian ratio and showing linkage with at least some other of the plurality of genetic markers;

e) measuring at least one of the properties in multiple progeny trees; and

f) correlating the presence of enhanced property with a least one marker identified in step d) as segregating in an essentially Mendelian ratio and showing linkage with at least some of the other markers, the correlation of the presence of enhanced properties with a marker indicating that the marker is associated with a genetic locus conferring enhanced; wherein the family of trees comprises a parent tree and its progeny.

The method in accordance with a preferred embodiment of the present invention, further comprising constructing a genetic linkage map of the parent tree using the plurality of genetic markers.

The method in accordance with a preferred embodiment of the present invention, wherein the genetic linkage map is a QTL map.

The method in accordance with a preferred embodiment of the present invention, wherein the genetic marker loci are restriction fragment length polymorphism (RFLPs) or PCR-fingerprint.

The method in accordance with a preferred embodiment of the present invention, wherein the restriction fragment length polymorphism (RFLPs) or PCR-fingerprint are correlated with a locus or with a quantitative traits loci (QTLs).

The method in accordance with a preferred embodiment of the present invention, wherein the parent tree is the seed parent tree to each of the progeny trees, root, leaf or cambium tissue from the progeny trees is assessed for the presence or absence of genetic markers in step c).

The method in accordance with a preferred embodiment of the present invention, wherein the parent tree is a species of Populus trichocarpa, Populus deltoides, Populus tremuloides or a hybrid thereof.

In accordance the present invention, there is provided a method of producing a plurality of clonal trees that have at least one enhanced property selected from the group consisting of fiber length, fiber coarseness, DBII (diameter at breast height), microfibril angle, density, pulp strength, pulp yield, lignin content, pitch propensity and calcium accumulation, which comprises the steps of:

a) obtaining a sexually mature parent tree exhibiting enhanced property relative to a value characteristic of the average of the genus;

b) obtaining a plurality of progeny trees of the parent tree by performing self or cross-pollination;

c) assessing multiple progeny tress for each of a plurality of genetic markers;

d) identifying genetic markers segregating in an essentially Mendelian ratio and showing linkage with at least some other of the plurality of genetic markers;

e) measuring at least one of the properties in multiple progeny trees;

f) correlating the presence of enhanced property with a least one marker identified in step d) as segregating in an essentially Mendelian ratio and showing linkage with at least some of the other markers;

g) selecting a progeny tree containing a marker identified in step f) as associated with a genetic locus conferring enhanced property; and

h) vegetatively propagating the progeny tree selected in step g) to produce a plurality of clonal trees, essentially all of the clonal trees exhibiting enhanced fiber length.

In accordance with the present invention, there is provided a stand of clonal enhanced property trees produced by the method of the present invention, the genome of the trees containing the same genetic marker associated with the enhanced property relative to a value characteristic of the average of the genus.

In accordance with the present invention, there is provided a method of producing a family of trees wherein at least about half exhibit at least of enhanced property selected from the group consisting of fiber length, fiber coarseness, DBII (diameter at breast height), microfibril angle, density, pulp strength, pulp yield, lignin content, pitch propensity and calcium accumulation, which comprises the steps of:

a) obtaining a sexually mature parent tree exhibiting enhanced property relative to a value characteristic of the average of the genus;

b) obtaining a plurality of progeny trees of the parent tree by performing self or cross-pollination;

c) assessing multiple progeny tress for each of a plurality of genetic markers;

d) identifying genetic markers segregating in an essentially Mendelian ratio and showing linkage with at least some other of the plurality of genetic markers;

e) measuring at least one of the properties in multiple progeny trees;

f) correlating the presence of enhanced fiber length with a least one marker identified in step d) as segregating in an essentially Mendelian ratio and showing linkage with at least some of the other markers;

g) selecting a progeny tree containing a marker identified in step f) as associated with a genetic locus conferring enhanced property; and

h) sexually propagating the progeny tree selected in step g) to produce a family of trees, at least about half of the family of trees containing a genetic locus conferring enhanced property and the family of trees exhibiting enhanced property.

In accordance with the present invention, there is provided a genetic map of QTLs of trees associated with enhanced properties as set forth in FIG. 30.

The genetic map in accordance with a preferred embodiment of the present invention, wherein the enhanced properties are selected from the group consisting of fiber length, fiber coarseness, DBII (diameter at breast height), microfibril angle, density, pulp strength, pulp yield, lignin content, pitch propensity and calcium accumulation.

In accordance with the present invention, there is provided a genetic marker of fiber length of trees, which comprises a 800 bp amplification product, wherein presence of the product in an amplified DNA sample from the trees is indicative of a short fiber length<0.92 mm and absence of the product is indicative of long fiber length>0.92 mm.

For the purpose of the present invention the following terms are defined below.

The term “Quantitative Trait locus (QTL)” is intended to mean the position(s) occupied on the chromosome by the gene(s) representing a particular trait. The various alternate forms of the gene—that is the alleles used in mapping—all reside at the same location.

The term “restriction fragment linked polymorphism (RFLP)” as used herein means a digestive enzymatic method for detecting localized differences in DNA sequence.

The term “random amplified polymorphic DNA (RAPD)” as used herein means a PCR based method for detecting localised differences in DNA sequence.

The term “polymerase chain reaction (PCR)” as used herein means a cyclical enzyme-mediated method for making large numbers of identical copies of a stretch of DNA using specific primers.

The term “hybrid thereof” as used herein means a progeny issued from the interbreeding of trees of different breeds, varieties or species especially as produced through tree-breeding for specific genetic and phenotypic characteristics. A hybrid thereof is derived by cross-breeding two different tree species.

The term “candidate gene” as used herein means a sequence of DNA representing a potential gene (an open reading frame, ORF) located within a QTL whose predicted functionality may partially or totally be causal to the given phenotypic trait associated with the QTL.

### BRIEF DESCRIPTION OF THE DRAWINGS

FIG. 1 illustrates SilviScan-2 analysis of hybrid poplar core 331-1062. Data indicate the expected increase in MFA from bark (mature wood zone) to pith (juvenile wood zone). Three scans were performed at resolutions of 1 mm, 2 mm and 5 mm.

FIG. 2A illustrates GC spectrum for acetone extractives from Populus tremuloides (quacking aspen);

FIG. 2B illustrates GC spectrum for hybrid poplar 331-1016 (F2 TD×TD cross);

FIG. 3 illustrates accept chips % vs. wood density for selected clones which is indicating no correlation;

FIG. 4 illustrates bulk density vs. chip density for hybrid poplar chips showing the expected strong correlation

FIG. 5 illustrates Kappa number vs. H-factor: clone 331-1136 which proved difficult to pulp is clearly distinct from the others;

FIG. 6 illustrates pulp yield vs. kappa number;

FIG. 7 illustrates Yield at kappa 17 vs. H-factor to kappa 17;

FIG. 8 illustrates chip density vs. H-factor to kappa 17;

FIG. 9 illustrates fiber coarseness vs. fiber length;

FIG. 10 illustrates chip density vs. fiber length;

FIG. 11 illustrates tensile index vs. bulk;

FIG. 12 illustrates histogram of tensile strength and bulk properties for the examined genotypes;

FIG. 13 illustrates tensile index development by PFI beating;

FIG. 14 illustrates tensile index vs. Canadian standard freeness;

FIG. 15 illustrates air resistance (Gurley) vs. sheet density;

FIG. 16 illustrates sheet density vs. Sheffield smoothness;

FIG. 17 illustrates scattering coefficient vs. Canadian standard freeness shows very poor correlation;

FIG. 18 illustrates handsheet deformations caused by calcium deposition;

FIG. 19 illustrates EDS characterization of vessel element mineral deposits;

FIG. 20 illustrates Electron micrograph of vessel element mineral deposition;

FIG. 21 illustrates unscreened Canadian standard freeness vs. specific refining energy exhibits low, medium and high refining energy demand envelopes at a given freeness value;

FIG. 22 illustrates uptake of NaOH and H₂O₂ vs. specific refining energy;

FIG. 23 illustrates mean chemical uptake vs. chip density;

FIG. 24 illustrates mean chemical uptake vs. tensile index at 200 mL;

FIG. 25 illustrates uptake vs. wood chip density;

FIG. 26 illustrates fines content vs. scattering coefficient indicating high levels of intraclonal variability;

FIG. 27 illustrates mean chemical uptake vs. scattering coefficient;

FIG. 28 illustrates roughness vs. freeness;

FIG. 29 illustrates Sheffield smoothness vs. tensile index; and

FIG. 30 illustrates genetic map of the hybrid poplar population produced using Mapmaker 3.0 and Mapmaker/QTL 1.1.

### DETAILED DESCRIPTION OF THE INVENTION

In accordance with the present invention, there is provided nucleic acid-based marker for tree phenotype prediction and method thereof.

Materials and Methods

Sample Sites

Sampling was conducted at the Washington State University Farm plantation site in Puyallup, Washington and at two commercial plantation sites in Northern Oregon at Clatskanie and Boardman. The pedigree sampled was founded in 1981 by interspecific hybridization between Populus trichocarpa (clone 93-968) and P. deltoides (clone ILL-129). Two siblings from the first hybrid generation (F1 family 53), 53-246 and 53-242, were crossed in 1988 to give rise to a family of second generation hybrids used for genetic mapping studies (F2 family 331). Unrooted cuttings of the P, F1 and 55 F2 clones were planted at the sites in a modified randomized complete block design at a 2×2 m spacing. At the time of sampling, the trees were seven (Puyallup) and five (Clatskanie, Boardman) years old.

Tree Sampling

Ten millimeters diameter increment cores were obtained at approximately breast height from 350 surviving trees (90 genotypes) within the pedigree. All cores were removed through the pith from bark to bark. For pilot Kraft pulping analyses, 25 stems were selected—based on the fiber properties and wood density phenotypic data—and harvested from the Puyallup site. The entire stem to a 1″ top size was recovered in each case. Genotyping experiments were performed on DNA extracted from 30 g of live tissue (leaf samples) obtained from each of the 90 sampled genotypes spanning the three growth sites.

Fiber Coarseness and Macerated Pulp Yield

Fibers for analysis were obtained from hand-chipped 10 mm increment cores using an acetic acid/hydrogen peroxide maceration technique whereby a known oven-dried (o.d.) weight of chips was first placed in a test tube, saturated with water then covered in maceration solution [1:1 mixture of glacial acetic acid: hydrogen peroxide (30% from stock bottle)]. These samples were then incubated in a dry heating block for 48 hrs at 60° C. The maceration solution was washed from the chips extensively using distilled water and the pulps disintegrated in a small Hamilton Beach mixer. A dilution series was then used to obtain representative samples of 10,000-20,000 fibers (corresponding to approximately 5 mg of macerated pulp) which were analyzed for length and coarseness values using a Kajaani FS-200 instrument and/or an OpTest Fiber Quality Analyzer. Maceration yields were calculated from oven-dried recovered pulps after fiber analysis.

Microfibril Angle

Microfibril angle (MFA) was measured on 45 whole increment core samples from the family 331 hybrid poplars. The cores were selected on the basis of sufficient size (>20 mm) and soundness of the wood. Prior to analysis, the cores were extracted in denatured ethanol for three days and dried. MFA was determined by SilviScan-2 analysis using scanning X-ray diffractometry [Evans, R. a variance approach to the X-ray diffractometric estimation of microfibril angle in wood. appita J. 52(4), 283-289 (1999)]. Acquisition time was set for 30 seconds to optimize signal to noise ratio and a single diffraction pattern was obtained for each sample to ensure that the entire length of the sample was represented. MFA was estimated from the standard deviation (S) of the 002 azimuthal diffraction profile where:

MFA=1.28(S²-36)^{1/2}

S and MFA are both measured in degrees.

Chemical Analyses—Lignin, Extractives (GCIMS)

1. Lignin

Lignin contents were determined for 90 genotypes sampled at the Puyallup growth site. The determinations were performed at the Paprican Pointe Claire facility according to TAPPI standard methods (T13 wd 74).

2. Extractives Preparation

The samples were ground in a Wiley Mill at 40 mesh and a 5-6 g o.d. aliquot of the ground wood was placed in a soxhlet thimble and continuously extracted with acetone for 6 hours. The resulting filtrate was concentrated by rotary evaporation and filtered through a pasteur pipette with glass wool, in order to remove any large particulates. The filtrate was then freeze dried, accurately weighed and the resulting crystals re-suspended in acetone to give a concentration of 5,000 ppm based on the total extractives yield. The internal standards, cholesterol palmitate and heptadeptanoic acid (C-17), were added to every one of the extracted samples, at a concentration of 200 ppm. The samples were then transferred to GC vials for analysis of fatty acids by GCMS, using a 10 m DB-XLB column (J&W). The set temperature program started out at 50° C. for 3 minutes, before ramping the temperature up to 340° C. at a rate of 10° C. per minute. This was then followed by maintaining the temperature at 340° C. for 30 minutes and again ramping up to 360° C. at a rate of 10° C. per minute. The injector temperature was held at 320° C. and a constant flow rate of 1.6 mL/minute was maintained. A solvent delay of 5 minutes was set up and data acquisition began at that point. In order for ion detection to occur, a compound table of known retention times was built. Peaks were detected by quantions (RIC) and integrated. Area ratios were determined relative to the internal standard, cholesterol palmitate.

The peaks were identified and integrated via the compound table that was constructed as a part of the MS data calculations [Fernandez, MP, Watson, PA, Breuil, C. Gas Chromatography-mass extractive compounds in quaking aspen. Journal of Chromatography A, 922(ER1-2): 225-233 (2001). The resulting area integrations from each spectrum were divided into the internal standard, cholesterol palmitate, to give a ratio. This relative number was then used on a peak specific basis (peak identification by retention time) as phenotypic data for genetic mapping experiments. The area of particular interest falls between 25 to 40 minutes and contains the waxes, sterols and steryl esters, the major components of pitch in wood.

Pulps Preparation

1. Wood Chip Preparation

Selected wood logs from the 25 hybrid poplar clones from the base up to a 1″ top diameter were debarked, slabbed (if necessary to reduce the diameter) on a portable Woodmizer LT-15 sawmill and chipped using a 36″ CM&E 10-knife industrial disc chipper. A portion of the chips were air-dried and later screened in a Wennberg chip classifier to obtain chips in the thickness range of 2-6 mm for chemical pulping. These accept chips were used in the kraft cooks. The remaining green chips were screened on a BM&H vibratory screen to remove over sized chips and fines prior to mechanical pulping.

2. Kraft Pulping

Three representative aliquots of air-dried accept chips from each of the samples were kraft pulped in bombs [45 g oven-dried (o.d.) charge] within a B-K micro-digester assembly. The cooking conditions were as follows:

Time to maximum temperature: 135 min

Maximum cooking temperature: 170° C.

Effective alkali, % OD weight of wood: 13%

% Sulphidity: 25%

Liquor to wood ratio: 5:1

H factor: 700-1400

All of the pulps produced were washed, oven-dried and weighed to determine pulp yield. Kappa number and black liquor residual effective alkali were determined by TAPPI standard procedures (T236 cm 85 and T625 respectively). From these results the optimum cooking conditions required to produce pulps at 17 Kappa number were estimated by fitting regression lines through each set of data (r²≧0.95). Large quantities of kraft pulp were subsequently produced in a 28 L Weverk laboratory digester. The pulps produced were disintegrated, washed and screened through an 8-cut screen plate.

A PFI mill was used to prepare 5-point beating curves for each pulp sample by refining at: 0, 1000, 3000, 6000 revolutions (CPPA Standard C.7). A disintegrator (CPPA Standard C.9P) and a stainless steel sheet machine were used for testing and forming all sets of handsheets (CPPA Standard C.4 and C.5). All physical and optical testing was performed in a constant temperature and humidity room, using CPPA standard methods.

3. Alkaline Peroxide Refiner Mechanical Pulping (APRMP)

Two-stage impregnation of twenty-four hybrid poplar chips samples was carried out using a Sunds Defibrator Prex impregnator with a 3:1 compression ratio.

Stage One

Chips were steamed at atmospheric pressure for 10 min to expel entrapped air from the chips and replace it with water vapour. Impregnation with a solution containing 0.25% DTPA (diethylenetriamine pentaacetic acid) was carried out in the Prex impregnator. This provided a chemical charge of 0.26% to 0.66% DTPA on o.d. wood.

Stage Two

First-stage impregnated chips were further impregnated with a solution containing 0.25% MgSO₄, 2.0% Na₂SiO₃, 2.35% NaOH and 1.5% H₂O₂. This resulted in chemical charges as follows:

| | |
|---|---|
| MgSO₄ applied, % o.d. wood: | 0.36 to 0.69 |
| Na₂SiO₃ applied, % o.d. wood: | 2.29 to 5.45 |
| NaOH applied, % o.d. wood: | 3.69 to 5.89 |
| H₂O₂ applied, % o.d. wood: | 1.72 to 3.76 |

After 60 min retention at 60° C. the side port of the preheater was opened to remove the impregnated chips for open-discharge refining in a 30.5 cm single-disc Sprout Waldron laboratory refiner to prepare alkaline peroxide refiner mechanical pulps (APRMP). Each chip sample was refined at three energy levels to give 72 APRMP pulps in the freeness range from 144 to 402 mL Csf. Immediately after first pass open-discharge refining the pulp stock was neutralized to pH 4.5-4.8. Wood chip density and chemical uptake of hybrid poplar chip samples are shown in Table XIX.

Other pertinent refining conditions are shown below:

| | |
|---|---|
| Plates | D2A507 |
| Number of passes | 2 to 4 depending upon freeness level |
| Nominal plate gap | 0.38 mm (first pass) |
| | 0.03 to 0.2 mm (subsequent passes) |
| Refining consistency | 18 to 23% o.d. pulp |

After latency removal, each pulp was screened on a 6-cut laboratory flat screen to determine screen rejects. Bauer-McNett fiber classifications on screened pulps were determined. Representative samples from each of the 72 pulp samples were analyzed for fiber length using a Kajaani FS-200 instrument. Handsheets were prepared with white water recirculation to minimize the loss of fines and tested for bulk, mechanical, and optical properties using CPPA standard methods. Handsheet roughness was measured in Sheffield units (SU).

Assessment of Calcium Accumulation

The nature of the observed kraft pulp handsheet deformations was explored by both light and electron microscopy and by energy-dispersive X-ray analysis. Wood chip deposits were characterized in similar fashion. The methodologies used have been described fully in a previous report.

Genetic Map Construction and QTL Mapping

The Populus genetic map used in this application, previously constructed using the same family 331 pedigree, consists of 342 RFLP, STS and RAPD markers and is described in [Bradshaw, H. D., Villar, M., Watson, B. D., Otto, K. G., and Stewart, S. “Molecular genetics of growth and development in Populus III. A genetic linkage map of a hybrid poplar composed of RFLP, STS and RAPD markers,” Theor. Appl. Genet. 89, 551-558 (1994)]. The 19 large linkage groups, corresponding closely to the 19 Populus chromosomes, were scanned for the phenotypic data obtained using the program MAPMAKER-QTL 1.1. Based on the scanned genome length and the distance between genetic markers, a logarithmic odds (LOD) significance threshold level of 2.9 was chosen (this ensures that the chance of a false positive QTL being detected is at most 5%). For more details on the QTL mapping procedure employed.

RAPD Analysis, Polymerase Chain Reaction (PCR) and Product Cloning

For each trait examined, QTL-associated markers were identified from the genetic map and were employed to generate polymorphic products from phenotyptically selected F2 generation individuals. Random Amplified Polymorphic DNA (RAPD) markers were purchased from Operon Technologies Inc. (Alameda, Calif., U.S.A.) and Restriction Fragment Linked Polymorphism (RFLP) markers were constructed from published sequence data by the Biotechnology Laboratory at the University of British Columbia.

Both types of markers were used in standard PCR reactions to generate polymorphic amplified product bands corresponding to the QTL-linked markers identified on the genetic map. PCR conditions were standard for RAPD analyses (H. D. Bradshaw, personal communication) and performed using rTaq polymerase (Amersham-Pharmacia) and a Techne Genius thermal cycler. Cycle conditions were as follows:

step

1: 94° C., 3 min

2: 94° C., 5 sec

3: 36° C., 30 sec

4: 72° C., 1 min

5: Repeat 2-4, 34×

6: 4° C., hold

PCR products from the phenotypically selected F2 generation individuals were separated on 1% agarose gels according to standard methods and polymorphic bands of the appropriate size were excised from the gels. Products were purified from the agarose using the Amersham-Pharmacia GFX PCR gel band purification kit and cloned into the Promega pGEM-T vector system (with supplied competent cells) according to manufacturers' protocols and standard blue/white selection cloning procedures on ampicillin agar. Cloned PCR products were prepared from transformed cells using the Promega Wizard Plus miniprep kit, again according to the manufacturers protocols, and were then sequenced at the Biotechnology Laboratory, University of British Columbia.

Results and Discussion

Fiber Coarseness and Macerated Pulp Yield

Fiber length and coarseness and macerated pulp yield data were obtained on core samples for each of the 350 trees sampled in the study using the pulp maceration technique and either the Kajaani FS-200 or the automated OpTest FQA instruments and are presented in Table I.

TABLE 1
Fiber length, coarseness and macerated pulp yield data

| Clone ID | Yield | Fiber Length | Coarseness | Site |
|---|---|---|---|---|
| 14-129 | 46.2 | 0.84 | 0.065 | Puyallup |
| 14-129 | 44.1 | 0.76 | 0.085 | |
| 93-968 | 50.8 | 0.99 | 0.095 | |
| 93-968 | 49.9 | 0.97 | 0.112 | |
| 93-968 | 50.5 | 0.98 | 0.102 | |
| 53-242 | 50.9 | 0.85 | 0.082 | |
| 53-242 | 47 | 0.83 | 0.069 | |
| 53-242 | 52.4 | 0.79 | 0.076 | |
| 53-246 | 50.9 | 0.83 | 0.065 | |
| 53-246 | 46.8 | 0.84 | 0.082 | |
| 331-1059 | 55.9 | 0.83 | 0.083 | |
| 331-1059 | 47.6 | 0.74 | 0.075 | |
| 331-1059 | 56.1 | 0.8 | 0.079 | |
| 331-1061 | 51.8 | 0.96 | 0.095 | |
| 331-1061 | 43.4 | 0.89 | 0.086 | |
| 331-1061 | 50.7 | 0.93 | 0.098 | |
| 331-1062 | 49.3 | 1.01 | 0.102 | |
| 331-1062 | 45.5 | 1 | 0.118 | |
| 331-1062 | 39.7 | 0.97 | 0.095 | |
| 331-1065 | 45.8 | 0.78 | 0.088 | |
| 331-1065 | 50.8 | 0.82 | 0.076 | |
| 331-1065 | 55.8 | 0.81 | 0.055 | |
| 331-1060 | 47.8 | 0.85 | 0.1037 | |
| 331-1060 | 51.8 | 0.81 | 0.089 | |
| 331-1064 | 55.9 | 0.98 | 0.064 | |
| 331-1064 | 48.9 | 0.96 | 0.083 | |
| 331-1067 | 52.3 | 0.82 | 0.064 | |
| 331-1067 | 47.6 | 0.87 | 0.063 | |
| 331-1067 | 53.2 | 0.87 | 0.066 | |
| 331-1069 | 49.6 | 0.89 | 0.095 | |
| 331-1069 | 56.1 | 0.99 | 0.1131 | |
| 331-1072 | 49.6 | 0.84 | 0.054 | |
| 331-1073 | 54.4 | 0.69 | 0.061 | |
| 331-1075 | 51.8 | 0.91 | 0.092 | |
| 331-1075 | 51 | 0.88 | 0.097 | |
| 331-1075 | 54.7 | 0.91 | 0.085 | |
| 331-1076 | 43.4 | 0.74 | 0.068 | |
| 331-1076 | 53.6 | 0.76 | 0.085 | |
| 331-1077 | 54.2 | 0.77 | 0.038 | |
| 331-1078 | 50.7 | 0.87 | 0.085 | |
| 331-1078 | 51 | 0.85 | 0.08 | |
| 331-1079 | 55.6 | 0.98 | 0.085 | |
| 331-1079 | 49.3 | 0.89 | 0.1 | |
| 331-1079 | 47.6 | 0.95 | 0.092 | |
| 331-1084 | 51.3 | 0.91 | 0.085 | |
| 331-1084 | 45.5 | 0.85 | 0.074 | |
| 331-1086 | 44.1 | 0.82 | 0.083 | |
| 331-1086 | 48.9 | 0.87 | 0.079 | |
| 331-1087 | 39.7 | 0.86 | 0.079 | |
| 331-1087 | 39.3 | 0.85 | 0.066 | |
| 331-1087 | 54.6 | 0.84 | 0.085 | |
| 331-1090 | 45 | 0.95 | 0.076 | |
| 331-1093 | 44.5 | 0.91 | 0.085 | |
| 331-1093 | 48.5 | 0.75 | 0.085 | |
| 331-1093 | 45.8 | 0.81 | 0.065 | |
| 331-1095 | 49.1 | 0.91 | 0.068 | |
| 331-1095 | 50.8 | 0.77 | 0.091 | |
| 331-1101 | 47.2 | 0.93 | 0.095 | |
| 331-1101 | 55.2 | 0.96 | 0.082# | |
| 331-1101 | 55.8 | 0.92 | 0.076 | |
| 331-1102 | 43.5 | 0.73 | 0.073 | |
| 331-1102 | 47.7 | 0.82 | 0.083 | |
| 331-1103 | 46.2 | 0.81 | 0.085 | |
| 331-1103 | 49.6 | 0.92 | 0.09 | |
| 331-1103 | 50.7 | 0.95 | 0.081 | |
| 331-1104 | 44.1 | 0.81 | 0.066 | |
| 331-1104 | 51.5 | 0.82 | 0.054 | |
| 331-1106 | 52 | 0.68 | 0.075 | |
| 331-1106 | 50.8 | 0.79 | 0.068 | |
| 331-1112 | 48.2 | 0.6 | 0.077 | |
| 331-1112 | 50.8 | 0.75 | 0.078 | |
| 331-1112 | 49.9 | 0.76 | 0.065 | |
| 331-1114 | 51.3 | 0.87 | 0.102 | |
| 331-1114 | 48.1 | 0.98 | 0.099 | |
| 331-1114 | 50.5 | 0.99 | 0.118 | |
| 331-1118 | 46.9 | 0.81 | 0.098 | |
| 331-1118 | 51 | 0.99 | 0.078 | |
| 331-1120 | 50.9 | 0.83 | 0.065 | |
| 331-1121 | 48.2 | 0.54 | 0.055 | |
| 331-1122 | 51.9 | 0.84 | 0.062 | |
| 331-1122 | 47 | 0.78 | 0.077 | |
| 331-1122 | 45.9 | 0.77 | 0.064 | |
| 331-1126 | 52.7 | 1.03 | 0.113 | |
| 331-1126 | 52.4 | 0.98 | 0.099 | |
| 331-1126 | 44 | 0.93 | 0.098 | |
| 331-1127 | 47.2 | 0.87 | 0.102 | |
| 331-1127 | 50.9 | 1.01 | 0.124 | |
| 331-1127 | 48.4 | 1.02 | 0.075 | |
| 331-1128 | 53.2 | 0.9 | 0.085 | |
| 331-1128 | 46.8 | 0.85 | 0.085 | |
| 331-1128 | 39.7 | 0.85 | 0.082 | |
| 331-1130 | 47.8 | 0.85 | 0.083 | |
| 331-1130 | 48.9 | 0.92 | 0.079 | |
| 331-1130 | 51.8 | 0.93 | 0.103 | |
| 331-1131 | 44.1 | 0.99 | 0.098 | |
| 331-1131 | 55.9 | 0.96 | 0.085 | |
| 331-1133 | 45.5 | 0.76 | 0.078 | |
| 331-1133 | 48.9 | 0.69 | 0.069 | |
| 331-1136 | 51.3 | 0.64 | 0.077 | |
| 331-1136 | 52.3 | 0.69 | 0.082 | |
| 331-1140 | 56.1 | 0.8 | 0.081 | |
| 331-1140 | 54.2 | 0.78 | 0.086 | |
| 331-1149 | 51 | 0.91 | 0.123 | |
| 331-1149 | 46.9 | 0.94 | 0.122 | |
| 331-1149 | 55.2 | 0.94 | 0.118 | |
| 331-1151 | 45.8 | 0.77 | 0.042 | |
| 331-1151 | 47.6 | 0.94 | 0.106 | |
| 331-1151 | 54.4 | 0.91 | 0.117 | |
| 331-1158 | 48.1 | 0.75 | 0.064 | |
| 331-1158 | 51 | 0.7 | 0.083 | |
| 331-1158 | 52 | 0.77 | 0.075 | |
| 331-1162 | 50.7 | 0.81 | 0.078 | |
| 331-1162 | 47.2 | 0.89 | 0.087 | |
| 331-1162 | 52.4 | 0.94 | 0.085 | |
| 331-1163 | 50.5 | 0.62 | 0.05 | |
| 331-1163 | 48.3 | 0.65 | 0.054 | |
| 331-1169 | 44.8 | 0.71 | 0.085 | |
| 331-1169 | 47.9 | 0.78 | 0.091 | |
| 331-1169 | 45.9 | 0.8 | 0.121 | |
| 331-1173 | 49.9 | 0.96 | 0.092 | |
| 331-1173 | 49.1 | 0.81 | 0.075 | |
| 331-1173 | 50.8 | 0.91 | 0.092 | |
| 331-1174 | 46.2 | 0.85 | 0.093 | |
| 331-1174 | 51.2 | 0.88 | 0.124 | |
| 331-1182 | 47.6 | 0.88 | 0.091 | |
| 331-1186 | 45.8 | 0.91 | 0.089 | |
| 331-1186 | 52.6 | 0.95 | 0.084 | |
| 331-1186 | 44.9 | 0.99 | 0.077 | |
| 331-1580 | 53.2 | 0.67 | 0.075 | |
| 331-1580 | 51.7 | 0.72 | 0.081 | |
| 331-1580 | 48.1 | 0.79 | 0.066 | |
| 331-1582 | 46.8 | 0.86 | 0.075 | |
| 331-1582 | 51.6 | 1.01 | 0.068 | |
| 331-1582 | 47.3 | 0.94 | 0.075 | |
| 331-1587 | 52.8 | 0.85 | 0.076 | |
| 331-1587 | 50.5 | 0.91 | 0.075 | |
| 14-1292.1 | 34.8 | 0.77 | 0.077 | Boardman - B |
| 14-1293.2 | 39.6 | 0.75 | 0.095 | Clatskanie - C |
| 14-1294.1B | 42.3 | 0.67 | 0.113 | |
| 93-9682.2 | 46.9 | 0.86 | 0.11 | |
| 93-9682.1 | 42.3 | 0.79 | 0.092 | |
| 93-9683.1 | 45.9 | 0.72 | 0.105 | |
| 93-9683.2 | 49 | 0.73 | 0.088 | |
| 93-9684.1B | 55.5 | 0.79 | 0.098 | |
| 93-9684.2B | 58.3 | 0.81 | 0.1 | |
| 53-2422.2 | 53.3 | 0.74 | 0.101 | |
| 53-2423.1 | 51 | 0.71 | 0.087 | |
| 53-2423.2 | 53.2 | 0.7 | 0.12 | |
| 53-2424.1B | 53.2 | 0.71 | 0.08 | |
| 53-2461.1 | 46.2 | 0.65 | 0.18 | |
| 53-2461.2 | 46.3 | 0.66 | 0.097 | |
| 53-2462.1 | 50.6 | 0.64 | 0.087 | |
| 53-2464.1B | 56.7 | 0.68 | 0.073 | |
| 10591.1 | 28 | 0.6 | 0.087 | |
| 10591.2 | 37.2 | 0.6 | 0.146 | |
| 10593.2B | 58.1 | 0.61 | 0.103 | |
| 10594.1B | 46.2 | 0.73 | 0.123 | |
| 10601.1 | 47.5 | 0.6 | 0.094 | |
| 10601.2 | 50 | 0.61 | 0.106 | |
| 10602.1 | 49.5 | 0.66 | 0.119 | |
| 10602.2 | 54.2 | 0.72 | 0.111 | |
| 10603.2B | 48.3 | 0.54 | 0.057 | |
| 10604.2B | 43.3 | 0.56 | 0.099 | |
| 10611.1 | 47.9 | 0.79 | 0.079 | |
| 10611.2 | 47.6 | 0.79 | 0.117 | |
| 10614.1B | 47.7 | 0.79 | 0.097 | |
| 10614.2B | 50 | 0.75 | 0.044 | |
| 10622.1 | 49.5 | 0.79 | 0.114 | |
| 10622.2 | 54.2 | 0.71 | 0.097 | |
| 10624.1B | 48.3 | 0.59 | 0.068 | |
| 10624.2B | 43.3 | 0.51 | 0.092 | |
| 10653.1 | 47.9 | 0.69 | 0.105 | |
| 10653.2 | 47.6 | 0.66 | 0.094 | |
| 10654.1B | 47.7 | 0.74 | 0.156 | |
| 10654.2B | 34.8 | 0.74 | 0.175 | |
| 10671.1 | 34.8 | 0.68 | 0.098 | |
| 10671.2 | 39.6 | 0.68 | 0.128 | |
| 10674.1B | 42.3 | 0.64 | 0.075 | |
| 10674.2B | 46.9 | 0.65 | 0.071 | |
| 10693.1 | 42.3 | 0.67 | 0.211 | |
| 10693.1B | 45.9 | 0.69 | 0.139 | |
| 10693.2B | 47.5 | 0.68 | 0.129 | |
| 10721.2 | 50 | 0.63 | 0.138 | |
| 10722.1 | 49.5 | 0.72 | 0.147 | |
| 10722.2 | 54.2 | 0.71 | 0.131 | |
| 10724.1B | 48.3 | 0.72 | 0.139 | |
| 10724.2B | 43.3 | 0.67 | 0.149 | |
| 10731.1 | 47.9 | 0.66 | 0.242 | |
| 10731.2 | 47.6 | 0.65 | 0.241 | |
| 10732.2 | 47.7 | 0.64 | 0.234 | |
| 10734.1B | 38 | 0.67 | 0.201 | |
| 10734.2B | 43.2 | 0.56 | 0.111 | |
| 10751.1 | 27.2 | 0.66 | 0.114 | |
| 10751.2 | 50.6 | 0.74 | 0.076 | |
| 10754.1B | 50 | 0.78 | 0.087 | |
| 10754.2B | 48.7 | 0.66 | 0.108 | |
| 10762.1 | 50.4 | 0.55 | 0.11 | |
| 10762.2 | 41 | 0.63 | 0.131 | |
| 10772.2 | 49.5 | 0.56 | 0.103 | |
| 10781.1 | 43.1 | 0.48 | 0.135 | |
| 10781.2 | 50.4 | 0.52 | 0.219 | |
| 10784.1B | 47.3 | 0.72 | 0.214 | |
| 10784.2B | 36.7 | 0.6 | 0.186 | |
| 10791.1 | 53 | 0.69 | 0.114 | |
| 10791.2 | 58.1 | 0.7 | 0.123 | |
| 10794.1B | 46.2 | 0.75 | 0.107 | |
| 10794.2B | 47.5 | 0.82 | 0.114 | |
| 10841.1 | 50 | 0.74 | 0.093 | |
| 10841.2 | 49.5 | 0.73 | 0.108 | |
| 10844.1B | 54.2 | 0.69 | 0.081 | |
| 10844.2B | 48.3 | 0.74 | 0.094 | |
| 10861.1 | 43.3 | 0.7 | 0.086 | |
| 10861.2 | 47.9 | 0.62 | 0.113 | |
| 10864.1B | 47.6 | 0.62 | 0.114 | |
| 10864.2B | 47.7 | 0.59 | 0.132 | |
| 10872.1 | 34.8 | 0.69 | 0.113 | |
| 10872.2 | 39.6 | 0.71 | 0.083 | |
| 10874.1B | 42.3 | 0.73 | 0.093 | |
| 10874.2B | 46.9 | 0.72 | 0.095 | |
| 10902.1 | 42.3 | 0.79 | 0.089 | |
| 10902.2 | 45.9 | 0.74 | 0.067 | |
| 10904.1B | 55.5 | 0.87 | 0.091 | |
| 10904.2B | 58.3 | 0.8 | 0.083 | |
| 10931.1 | 53.3 | 0.62 | 0.099 | |
| 10931.2 | 51 | 0.58 | 0.075 | |
| 10934.1B | 53.2 | 0.67 | 0.095 | |
| 10934.2B | 53.2 | 0.63 | 0.086 | |
| 10951.1 | 46.2 | 0.63 | 0.093 | |
| 10951.2 | 46.3 | 0.8 | 0.109 | |
| 10954.1B | 50.6 | 0.77 | 0.084 | |
| 10954.2B | 56.7 | 0.62 | 0.082 | |
| 11011.1 | 28 | 0.76 | 0.093 | |
| 11012.2 | 37.2 | 0.73 | 0.104 | |
| 11014.2B | 58.1 | 0.74 | 0.099 | |
| 11021.1 | 46.2 | 0.69 | 0.109 | |
| 11021.2 | 46.2 | 0.68 | 0.081 | |
| 11023.1B | 47.5 | 0.7 | 0.093 | |
| 11031.1 | 50 | 0.74 | 0.084 | |
| 11031.2 | 49.5 | 0.73 | 0.086 | |
| 11034.1B | 54.2 | 0.74 | 0.091 | |
| 11034.2B | 48.3 | 0.85 | 0.09 | |
| 11041.1 | 43.3 | 0.73 | 0.113 | |
| 11041.2 | 47.9 | 0.74 | 0.073 | |
| 11044.1B | 47.6 | 0.72 | 0.101 | |
| 11044.2B | 47.7 | 0.7 | 0.087 | |
| 11121.1 | 38 | 0.5 | 0.12 | |
| 11123.1 | 43.2 | 0.62 | 0.08 | |
| 11124.2B | 27.2 | 0.38 | 0.18 | |
| 11142.1 | 50.6 | 0.74 | 0.097 | |
| 11142.2 | 50 | 0.78 | 0.087 | |
| 11144.1B | 48.7 | 0.76 | 0.073 | |
| 11144.2B | 50.4 | 0.86 | 0.087 | |
| 11181.1 | 41 | 0.6 | 0.146 | |
| 11182.1 | 49.5 | 0.68 | 0.103 | |
| 11184.1B | 43.1 | 0.56 | 0.123 | |
| 11184.2B | 50.4 | 0.6 | 0.094 | |
| 11201.1 | 50.9 | 0.65 | 0.106 | |
| 11201.2 | 47.3 | 0.55 | 0.119 | |
| 11211.1 | 49.3 | 0.59 | 0.111 | |
| 11214.1B | 46.1 | 0.64 | 0.057 | |
| 11214.2B | 45.4 | 0.66 | 0.099 | |
| 11221.1 | 44.1 | 0.63 | 0.079 | |
| 11221.2 | 49.1 | 0.62 | 0.117 | |
| 11223.2B | 44 | 0.59 | 0.097 | |
| 11224.2B | 51.2 | 0.57 | 0.044 | |
| 11261.2 | 44.3 | 0.78 | 0.114 | |
| 11262.1 | 51.3 | 0.82 | 0.097 | |
| 11264.1B | 47.3 | 0.86 | 0.068 | |
| 11264.2B | 36.7 | 0.8 | 0.092 | |
| 11271.1 | 46.1 | 0.69 | 0.105 | |
| 11271.2 | 45.4 | 0.71 | 0.094 | |
| 11274.1B | 46.6 | 0.66 | 0.156 | |
| 11274.2B | 43.2 | 0.62 | 0.175 | |
| 11282.1 | 35 | 0.79 | 0.098 | |
| 11282.2 | 52.4 | 0.78 | 0.128 | |
| 11284.1B | 50 | 0.82 | 0.075 | |
| 11284.2B | 39 | 0.87 | 0.071 | |
| 11302.1 | 39.6 | 0.72 | 0.211 | |
| 11302.2 | 42.3 | 0.66 | 0.139 | |
| 11311.1 | 46.9 | 0.71 | 0.129 | |
| 11311.2 | 42.3 | 0.73 | 0.138 | |
| 11312.1 | 45.9 | 0.64 | 0.147 | |
| 11313.2B | 27.2 | 0.67 | 0.131 | |
| 11334.1B | 50.6 | 0.64 | 0.139 | |
| 11334.2B | 44.3 | 0.65 | 0.149 | |
| 11361.1 | 45.4 | 0.6 | 0.242 | |
| 11361.2 | 44.1 | 0.56 | 0.241 | |
| 11362.2 | 49.1 | 0.53 | 0.234 | |
| 11401.1 | 44 | 0.55 | 0.201 | |
| 11402.1 | 51.2 | 0.61 | 0.111 | |
| 11402.2 | 44.3 | 0.62 | 0.114 | |
| 11404.1B | 51.3 | 0.64 | 0.076 | |
| 11404.2B | 51.4 | 0.74 | 0.087 | |
| 11491.1 | 37.1 | 0.74 | 0.108 | |
| 11491.2 | 49.4 | 0.79 | 0.11 | |
| 11492.1 | 50.8 | 0.68 | 0.131 | |
| 11494.1B | 35.5 | 0.65 | 0.103 | |
| 11494.2B | 46.5 | 0.7 | 0.135 | |
| 11511.1 | 47.2 | 0.59 | 0.219 | |
| 11511.2 | 46.6 | 0.71 | 0.214 | |
| 11511.22 | 43.2 | 0.69 | 0.186 | |
| 11514.1B | 35 | 0.8 | 0.114 | |
| 11581.1 | 52.4 | 0.62 | 0.123 | |
| 11581.2 | 50 | 0.67 | 0.107 | |
| 11583.1B | 39 | 0.61 | 0.114 | |
| 11583.2B | 51.4 | 0.77 | 0.093 | |
| 11584.2B | 37.1 | 0.59 | 0.108 | |
| 11621.2 | 49.4 | 0.7 | 0.081 | |
| 11622.1 | 50.8 | 0.69 | 0.094 | |
| 11624.1B | 35.5 | 0.48 | 0.086 | |
| 11631.1 | 46.5 | 0.61 | 0.113 | |
| 11631.2 | 47.2 | 0.64 | 0.114 | |
| 11634.1B | 46.6 | 0.48 | 0.132 | |
| 11634.2B | 43.2 | 0.54 | 0.113 | |
| 11653.1 | 35 | 0.53 | 0.083 | |
| 11691.1 | 52.4 | 0.63 | 0.093 | |
| 11691.2 | 49.3 | 0.55 | 0.095 | |
| 11694.1B | 58.9 | 0.66 | 0.089 | |
| 11694.2B | 52.2 | 0.69 | 0.067 | |
| 11732.1 | 49.8 | 0.6 | 0.091 | |
| 11732.2 | 46.5 | 0.65 | 0.083 | |
| 11733.1 | 46.6 | 0.56 | 0.099 | |
| 11733.2 | 50.3 | 0.61 | 0.075 | |
| 11734.1B | 47.6 | 0.69 | 0.095 | |
| 11734.2B | 48.4 | 0.67 | 0.086 | |
| 11741.1 | 52.7 | 0.68 | 0.093 | |
| 11741.2 | 48 | 0.57 | 0.109 | |
| 11862.1 | 50.9 | 0.74 | 0.084 | |
| 11862.2 | 47.3 | 0.7 | 0.082 | |
| 15803.1 | 49.3 | 0.6 | 0.093 | |
| 15803.2 | 46.1 | 0.53 | 0.104 | |
| 15804.1B | 45.4 | 0.62 | 0.099 | |
| 15804.2B | 44.1 | 0.65 | 0.109 | |
| 15823.1 | 49.1 | 0.78 | 0.081 | |
| 15823.2 | 44 | 0.74 | 0.093 | |
| 15823.1B | 51.2 | 0.72 | 0.084 | |
| 15823.2B | 44.3 | 0.66 | 0.086 | |
| 15871.1 | 51.3 | 0.69 | 0.091 | |
| 15871.2 | 47.3 | 0.65 | 0.09 | |
| 15874.1B | 36.7 | 0.49 | 0.113 | |
| 15874.2B | 53 | 0.63 | 0.073 | |

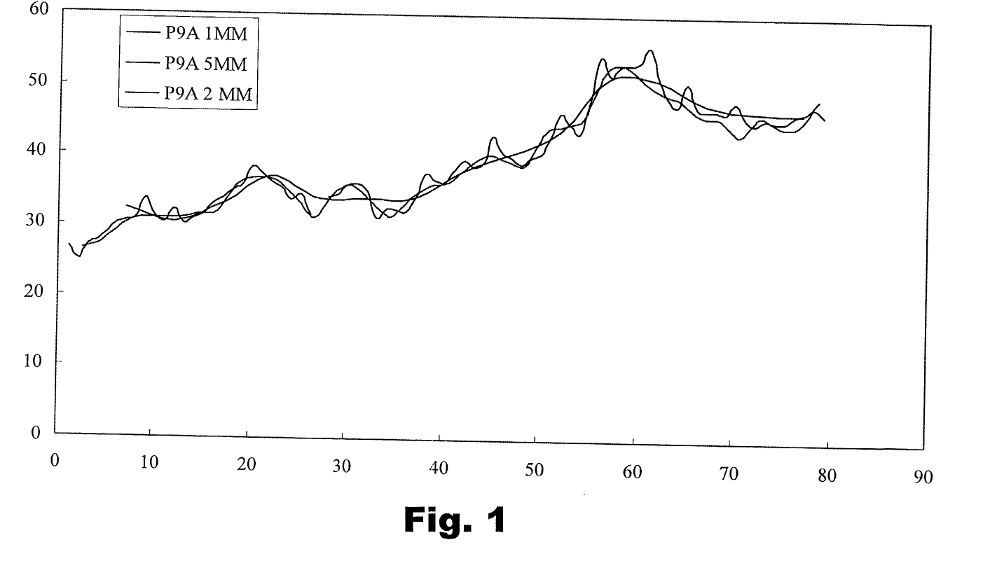

Previous experiments have shown no difference in the fiber properties analyses of poplar samples between these two instruments [Robertson, G., Olson, J., Allen, P., Chan, B. and Seth, R. “Measurement of fiber length, coarseness and shape with the fiber quality analyzer”. TAPPI J. 82(10), 93-98 (1999)]. The outermost ring (age 7) data are presented in Table I. Microfibril angle data for the outermost ring of each core (i.e. age 7), obtained using the SilvisScan-2 technique, are also presented in Table II. FIG. 1 shows the results of a typical SilviScan-2 analysis of an increment core sample from bark to pith at different levels of scanning resolution.

TABLE II
Microfibril angle data for hybrid poplars at age 7.

| TREE | MFA |
|---|---|
| 331- | Ring 7 data |
| 1060 | 29.22 |
| 1063 | 26.15 |
| 1064 | 30.45 |
| 1065 | 27.13 |
| 1067 | 32.09 |
| 1069 | 29.09 |
| 1072 | 30.06 |
| 1073 | 32.24 |
| 1075 | 33.55 |
| 1076 | 34.60 |
| 1078 | 29.58 |
| 1079 | 31.25 |
| 1080 | 23.40 |
| 1082 | 28.43 |
| 1084 | 28.90 |
| 1095 | 30.58 |
| 1101 | 25.48 |
| 1103 | 28.03 |
| 1104 | 35.26 |
| 1114 | 21.56 |
| 1120 | 25.75 |
| 1122 | 17.76 |
| 1126 | 26.14 |
| 1127 | 33.37 |
| 1128 | 25.15 |
| 1130 | 25.87 |
| 1131 | 25.30 |
| 1140 | 24.98 |
| 1149 | 28.42 |
| 1151 | 28.59 |
| 1158 | 25.92 |
| 1169 | 26.54 |
| 1174 | 25.25 |
| 1186 | 27.01 |
| 1580 | 38.19 |
| 1590 | 20.84 |
| 1591 | 30.02 |
| 1592 | 26.09 |
| 1593 | 26.51 |

Significant variability is seen for all three traits—fiber coarseness ranges from 0.042 mg/m to 0.124 mg/m; microfibril angle from 17.8° to 38.2°; maceration yield from 27.2% to 56.1%. Results of the Mapmaker-QTL 1.1 analysis of the data are shown in Table ll. One significant QTL has been found for fiber coarseness, one low significance QTL for microfibril angle and four for macerated pulp yield. The QTL for each fiber property are concident and one of the QTL for maceration yield (P1027_P192/R) is coincident with the low significance QTL detected for Kraft pulp yield (Table VI). These regions may, therefore, represent particularly important areas of the genome for pulp and paper properties.

TABLE III
Significant QTL detected for each examined property

| Trait | Marker/Linkage | LOD Score* | Phen %^{‡} | Length/cM | Weight | Dom. |
|---|---|---|---|---|---|---|
| Fiber | I14_09-F15_10/E | 3.49 | 55.9 | 37.3 | 72.794 | −79.906 |
| Coarseness | | | | | | |
| Microfibril | I14_09-F15_10/E | 2.38* | 39.8 | 37.3 | 0.9445 | 4.4460 |
| angle | | | | | | |
| Maceration | P1258-P75/C | 3.50 | 68.8 | 3.3 | −6.3878 | 6.4285 |
| yield | | | | | | |
| | I17_04-P1275/J | 3.18 | 75.4 | 15.4 | −5.3740 | 7.8547 |
| | P1218-G02_11/J | 4.26 | 73.4 | 13.8 | −5.7903 | 7.9257 |
| | P1027-P192/R | 2.98 | 50.0 | 0.0 | −2.8721 | 5.7712 |

LOD - logarithmic odds score;
^{‡}Phen. % - phenotypic variance explained by the QTL detected; length - recombination distance between genetic markers (in centimorgans, cM); weight and dominance measure the comparative effects of the P. trichocarpa and P. deltoides alleles on the phenotype.
*Low significance QTL reported due to location.

Lignin Composition

Data for the lignin compositional analyses undertaken on the core samples are presented in Table IV.

TABLE IV
Lignin contents for the harvested stems

| Clone | Lignin (%) |
|---|---|
| 14-129 | 24.56 |
| 93-968 | 25.57 |
| 53-242 | 23.31 |
| 53-246 | 24.50 |
| 331-1059 | 24.89 |
| 331-1061 | 25.75 |
| 331-1062 | 24.78 |
| 331-1075 | 24.87 |
| 331-1093 | 25.43 |
| 331-1118 | 23.99 |
| 331-1122 | 24.27 |
| 331-1126 | 23.38 |
| 331-1136 | 24.56 |
| 331-1162 | 22.93 |
| 331-1186 | 24.71 |

These phenotypic data were used in a Mapmaker-QTL 1.1 genetic mapping experiment which resulted in the identification of a single, significant QTL for lignin content (shown in Table V). Due to the extensive industrial and academic interest in the genetic control of this particular woody plant trait, many candidate genes for this region—primarily from the lignin biosynthetic pathway—have already been sequenced, a fact which may enable the rapid characterization of this QTL.

TABLE V
Significant QTL detected for lignin content

| Trait | Marker/Linkage | LOD Score | Phen % | Length/cM | Weight | Dom. |
|---|---|---|---|---|---|---|
| Lignin content | P757-P867/P | 3.32 | 24.7 | 16.7 | 0.5463 | −0.0099 |

Extractives Content—GC/MS Analysis

TABLE VI
Significant QTL detected for individual extractives peaks

| Trait | | | | | | |
|---|---|---|---|---|---|---|
| Compound | Marker/Linkage | LOD Score | Phen % | Length/cM | Weight | Dom. |
| Beta- | P1277-P12612/A | 9.84 | 83.3 | 14.7 | 4.7882 | −5.8067 |
| sitosterol | | | | | | |
| (r.t. 25.831) | P856-A18_06/I | 7.97 | 81.3 | 14.0 | 4.9972 | −5.5280 |
| | win8-G04_20/I | 10.47 | 81.3 | 27.0 | 5.0064 | −5.5178 |
| | P1202-P1221/O | 5.60 | 80.7 | 15.8 | −5.3093 | −4.9808 |
| Sterol | P1277-P12612/A | 5.03 | 69.4 | 14.7 | −0.9132 | −1.1720 |
| (r.t. 25.912) | P1011-C04_04/A | 5.70 | 68.8 | 23.5 | −0.9541 | −1.1478 |
| | P1322-P1310/A | 4.12 | 67.6 | 12.2 | −1.0231 | −0.9421 |
| | P1074-G12_15/B | 5.76 | 65.1 | 19.7 | −1.5614 | −1.4403 |
| | P44-P1054/B | 6.04 | 65.4 | 4.4 | −1.5744 | −1.4237 |
| | H12_03-P1196/B | 3.71 | 58.8 | 8.8 | −1.2545 | −1.0949 |
| | win8-G04_20/I | 5.16 | 64.7 | 27.0 | 1.5482 | −1.4744 |
| | G13_17-C10_21/I | 5.91 | 64.4 | 14.0 | 1.4861 | −1.5144 |
| | P65-P1203/J | 4.86 | 64.6 | 9.1 | 1.5060 | −1.5576 |
| | B15_17-P216/X | 2.97 | 31.5 | 0.4 | −0.5213 | −0.6455 |
| Sterol | win8-G04_20/I | 9.06 | 72.2 | 27.0 | 3.8061 | −3.6553 |
| (r.t. 25.917) | G13_17-C10_21/I | 9.20 | 72.0 | 14.0 | 3.7236 | −3.8242 |
| | I17_04-P1275/J | 8.86 | 72.2 | 15.4 | 3.8034 | −3.6422 |
| | P773-P1055/J | 7.17 | 72.2 | 3.9 | 3.8033 | −3.6495 |
| | P65-P1203/J | 9.21 | 72.0 | 9.1 | 3.7858 | −3.6910 |
| | P1218-G02_11/J | 9.55 | 71.9 | 13.8 | 3.7620 | −3.7391 |
| Sterol | P1277-P12612/A | 12.12 | 90.1 | 14.7 | −0.1879 | −0.3996 |
| (r.t. 26.319) | H19_08-E14_15/C | 6.53 | 81.7 | 19.7 | 0.3026 | −0.2430 |
| | P12182-P1049/C | 5.17 | 75.3 | 19.0 | −0.2181 | −0.2372 |
| | P13292-P1043/M | 6.27 | 79.2 | 12.0 | −0.2791 | −0.2991 |
| | P46-F15_18/X | 8.18 | 80.3 | 17.9 | −0.2996 | −0.2567 |
| | E18_05-P12743/X | 5.00 | 80.3 | 11.5 | −0.3007 | −0.2567 |
| | P1064-B15_17/X | 7.97 | 81.2 | 26.6 | −0.3044 | −0.2468 |
| Sterol/triter | P1277-P12612/A | 5.30 | 80.2 | 14.7 | 0.0157 | −0.1606 |
| pene | | | | | | |
| (r.t. 26.417) | H19_08-E14_15/C | 6.53 | 80.0 | 19.7 | 0.0858 | −0.0726 |
| | P12182-P1049/C | 3.20 | 77.1 | 19.0 | −0.0730 | −0.1091 |
| | P1018-P12242/E | 4.80 | 80.2 | 16.9 | −0.0705 | −0.0957 |
| | P1064-B15_17/X | 3.14 | 80.2 | 26.6 | −0.0782 | −0.0829 |
| Sterol | I14_09-F15_10/E | 3.35 | 65.3 | 37.3 | 0.1014 | −0.0849 |
| (r.t. 27.818) | I17_04-P1275/J | 3.46 | 63.9 | 15.4 | 0.0967 | −0.0985 |
| | P1218-G02_11/J | 4.34 | 63.5 | 13.8 | 0.0959 | −0.1006 |
| | E18_15-C01_16/M | 3.15 | 68.7 | 22.1 | −0.1074 | −0.0778 |
| Sterol/triter | P1277-P12612/A | 18.15 | 95.3 | 14.7 | 1.6108 | −1.6355 |
| pene | | | | | | |
| (r.t. 28.218) | P1011-C04_04/A | 18.99 | 97.3 | 23.5 | 1.5192 | −1.7546 |
| | P1291-P1267/L | 18.13 | 95.5 | 12.9 | 1.5951 | −1.6614 |
| Triterpene/ | P1145-G08_09/M | 3.96 | 78.4 | 12.7 | −2.6340 | −2.3716 |
| ester | | | | | | |
| (r.t. 37.833) | E18_15-C01_16/M | 3.76 | 77.1 | 22.1 | 2.5955 | −3.5004 |
| | P1064-B15_17/X | 4.72 | 81.1 | 26.6 | −2.6098 | −3.6878 |
| Triglyceride | P11642-P1145/M | 3.13 | 56.3 | 4.5 | −1.3120 | −2.0510 |
| (r.t. 40.084) | | | | | | |

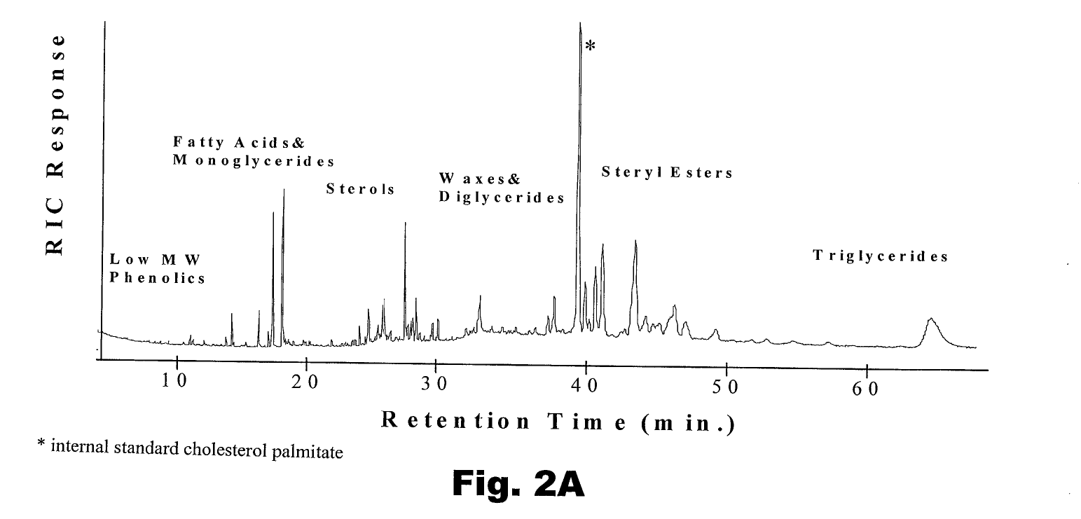

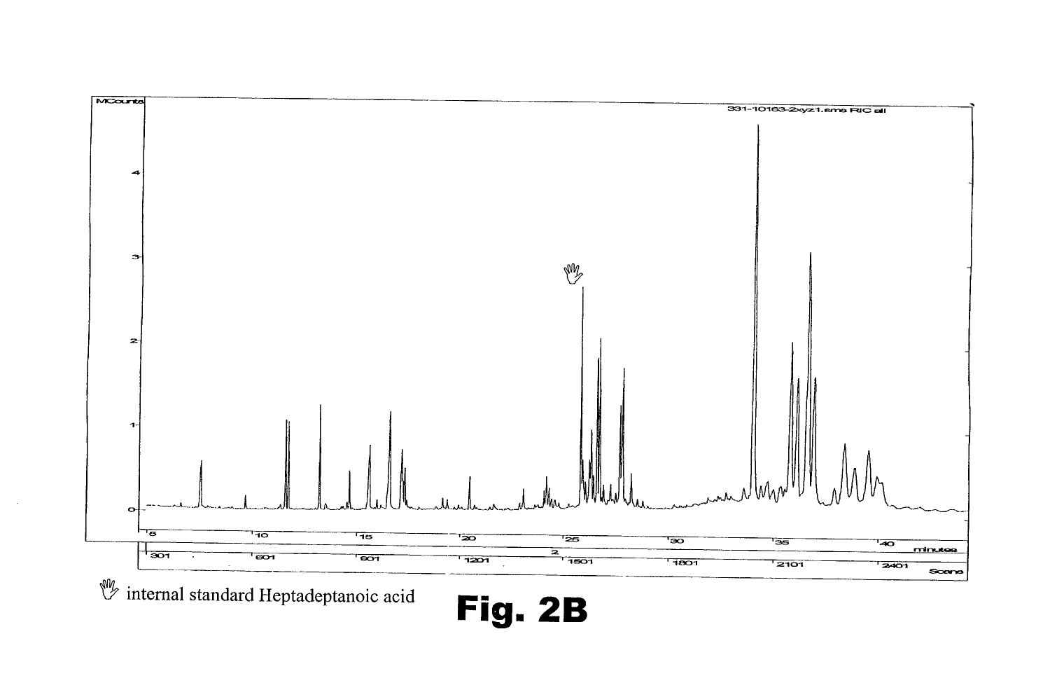

The GCMS method used for compound analysis was that developed and optimized by Fernandez et al. for the analysis of aspen (P. tremuloides) extractives. Peaks were identified via retention time and ion masses. The area of particular interest in the spectrum—containing the sterols and assorted waxes, compounds which are implicated in pitch formation propensity—was delineated as shown in FIG. 2A, at retention times greater than 25 min. The similarity between this aspen spectrum and those obtained from the hybrid poplar clones—a typical spectrum is shown in FIG. 2B—allowed the extrapolation of peak identification table data to the mapping population clones. Identified compounds were quantified, ratio numbers were obtained relative to the internal standard and were then used for QTL experiments. Significant QTL for extractives peaks are presented in Table V.

To date, this application has successfully identified a number of QTL that contain genes involved in the control of sterol and steryl ester content/synthesis in this family of hybrid poplars. The fact that several QTL have been independently detected for a number of related compounds provides strong evidence that the synthesis of a suite of related compounds is controlled by the same discrete genetic regions (implying the existence of a biosynthetic pathway) and that these QTL in particular may be regarded as non-spurious detections. These results both confirm and extend the conclusions of previous research describing clonal-based variation of extractives content in a natural population of aspen (P. tremuloides).

Chipping and Chip Quality of Hybrid Poplar Stems

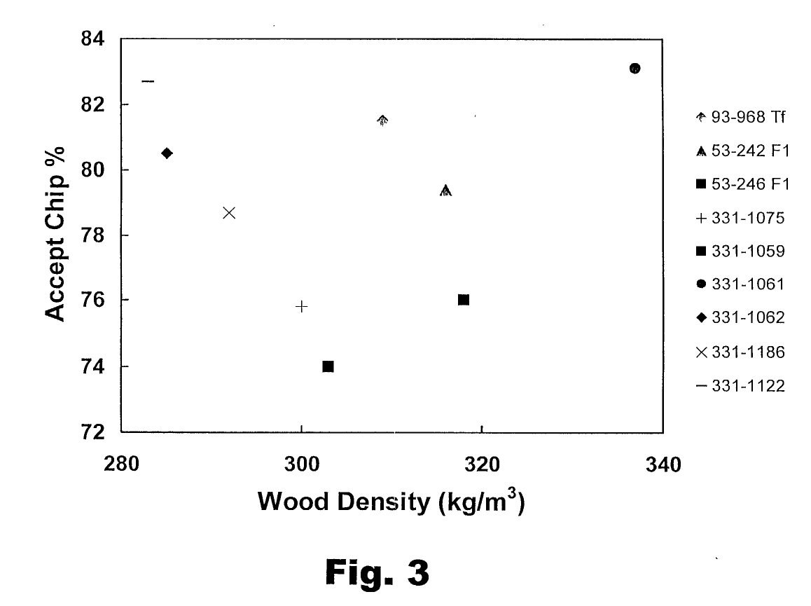

Whole logs of selected hybrid poplar clones were debarked and chipped as described in the experimental section. The wood density and chip quality of selected clones are presented in Table VII. Attempted correlations between the accept chip fraction and the wood density were unsuccessful (FIG. 3).

TABLE VII
Wood density and Chip Quality of Selected Clones

| | 93- | 53- | 53- | 331- | 331- | 331- | 331- | 331- | 331- |
|---|---|---|---|---|---|---|---|---|---|
| | 968 | 242 | 246 | 1059 | 1061 | 1062 | 1075 | 1122 | 1186 |
| Wood Density (kg/m³) | 309 | 316 | 318 | 303 | 337 | 285 | 300 | 283 | 292 |
| 45 mm round (%) | 0.9 | 4.3 | 4.5 | 2.9 | 1.4 | 1.4 | 2.9 | 0.2 | 1.8 |
| 8 mm slot (%) | 15.2 | 15.1 | 18.4 | 21.8 | 9.8 | 16.5 | 20.0 | 14.2 | 17.1 |
| 7 mm round (%) | 81.5 | 79.4 | 76.0 | 74.0 | 83.1 | 80.5 | 75.8 | 82.7 | 78.7 |
| 3 mm round (%) | 2.0 | 1.0 | 0.8 | 1.0 | 2.5 | 1.2 | 0.8 | 2.2 | 1.8 |
| Fines (%) | 0.6 | 0.4 | 0.4 | 0.4 | 0.5 | 0.5 | 0.5 | 0.7 | 0.6 |

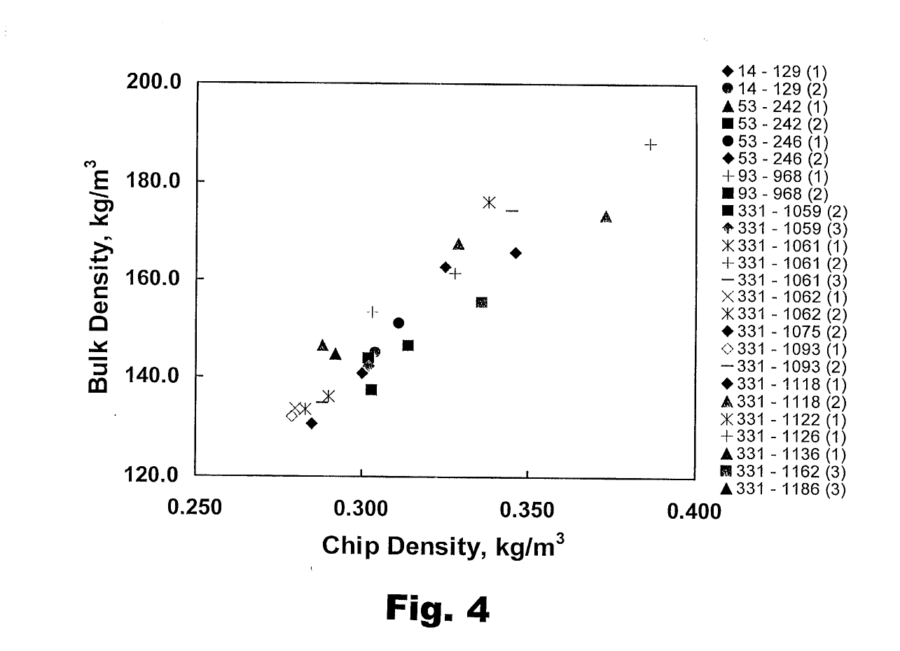

FIG. 4 shows a plot of chip density against bulk density (Table VIII) for the sampled stems.

TABLE VIII
Hybrid Poplar Chip Density And Chip Packing Density
(Bulk Density) Puyallup, Washington Site

| | Chip Density | Bulk Density |
|---|---|---|
| Sample Air Dried Chips | Kg/m³ | Kg/m³ |
| 14-129 (1) | 0.285 | 130.7 |
| 14-129 (2) | 0.304 | 145.1 |
| 53-242 (1) | 0.329 | 167.5 |
| 53-242 (2) | 0.302 | 143.9 |
| 53-246 (1) | 0.311 | 151.0 |
| 53-246 (2) | 0.325 | 162.6 |
| 93-968 (1) | 0.303 | 153.3 |
| 93-968 (2) | 0.314 | 146.5 |
| 331-1059 (2) | 0.303 | 137.5 |
| 331-1059 (3) | 0.302 | 142.3 |
| 331-1061 (1) | 0.338 | 176.1 |
| 331-1061 (2) | 0.328 | 161.4 |
| 331-1061 (3) | 0.345 | 174.3 |
| 331-1062 (1) | 0.280 | 133.8 |
| 331-1062 (2) | 0.290 | 136.2 |
| 331-1075 (2) | 0.300 | 140.8 |
| 331-1093 (1) | 0.279 | 132.1 |
| 331-1093 (2) | 0.288 | 134.8 |
| 331-1118 (1) | 0.346 | 165.7 |
| 331-1118 (2) | 0.373 | 173.3 |
| 331-1122 (1) | 0.283 | 133.5 |
| 331-1126 (1) | 0.386 | 188.0 |
| 331-1136 (1) | 0.288 | 146.5 |
| 331-1162 (3) | 0.336 | 155.4 |
| 331-1186 (3) | 0.292 | 144.7 |

Note:
Chip Thickness = 2-6 mm
Bulk density was done on air dried chips

The two parameters are related by a Pearson correlation coefficient of 0.86 (p=0.000). Higher density chips, such as those obtained from clone 331-1061, are more desirable as they pack better into kraft pulp digesters and mechanical pulp mill plug screw feeders thus ensuring maximum mill production rates. If these clones were to be ranked on the basis of chip value and quality (i.e. low oversized, pins and fines fractions), clones 331-1061, 331-1122, parent 93-968 and triploid 331-1062 would be considered superior material.

Kraft Pulping and Testing

1. Pulping Data

The 25 hybrid poplar trees (comprising 15 distinct genotypes) were chemically pulped according to the conditions outlined above and handsheets were prepared from the corresponding pulps. Calculated data for pulping to Kappa 17, derived from Table IX, are presented in Table X.

TABLE IX
Hybrid Poplar Exploratory Kraft Pulping Data (whole log chip samples)

| Sample | Kappa | % Unsc'd Yield | H Factor | % Res. EA | % EA Consumed | % Rejects |
|---|---|---|---|---|---|---|
| 14- | 27.1 | 55.9 | 800 | 3.0 | 10.0 | 0.7 |
| 129(1) | | | | | | |
| | 17.9 | 54.9 | 1100 | 2.8 | 10.2 | trace |
| | 15.6 | 53.8 | 1400 | 2.5 | 10.5 | 0.1 |
| 14- | 32.2 | 57.6 | 700 | 3.1 | 9.9 | 4.7 |
| 129(2) | | | | | | |
| | 23.1 | 55.1 | 1000 | 2.6 | 10.4 | 1.1 |
| | 17.5 | 53.6 | 1400 | 2.2 | 10.8 | 0.1 |
| 331- | 30.0 | 56.5 | 700 | 2.7 | 10.3 | 3.2 |
| 1059(2) | | | | | | |
| | 19.6 | 54.8 | 1000 | 2.3 | 10.7 | 0.3 |
| | 15.2 | 54.1 | 1400 | 2.1 | 10.9 | 0.2 |
| 331- | 24.6 | 55.4 | 800 | 2.5 | 10.5 | 0.4 |
| 1059(3) | | | | | | |
| | 17.8 | 54.1 | 1100 | 2.2 | 10.8 | 0.2 |
| | 14.9 | 53.6 | 1400 | 2.0 | 11.0 | 0.4 |
| 331- | 28.8 | 54.9 | 800 | 2.4 | 10.6 | 1.0 |
| 1061(1) | | | | | | |
| | 20.9 | 53.9 | 1100 | 2.2 | 10.8 | 0.1 |
| | 17.9 | 52.8 | 1400 | 2.0 | 11.0 | trace |
| 331- | 27.9 | 55.5 | 800 | 2.5 | 10.5 | 1.5 |
| 1061(2) | | | | | | |
| | 17.5 | 54.2 | 1100 | 2.3 | 10.7 | trace |
| | 15.0 | 53.4 | 1400 | 2.1 | 10.9 | trace |
| 331- | 25.3 | 55.2 | 800 | 2.5 | 10.5 | 0.5 |
| 1061(3) | | | | | | |
| | 18.3 | 54.6 | 1100 | 2.3 | 10.7 | 0.2 |
| | 15.3 | 53.5 | 1400 | 2.0 | 11.0 | 0.3 |
| 331- | 25.7 | 55.5 | 800 | 2.7 | 10.3 | 2.4 |
| 1062(1) | | | | | | |
| | 18.9 | 53.7 | 1100 | 2.3 | 10.7 | 0.5 |
| | 14.8 | 53.2 | 1400 | 2.1 | 10.9 | trace |
| 331- | 25.2 | 54.6 | 800 | 2.5 | 10.5 | 0.9 |
| 1062(2) | | | | | | |
| | 18.0 | 53.0 | 1100 | 2.2 | 10.8 | 0.4 |
| | 15.1 | 52.6 | 1400 | 2.1 | 10.9 | trace |
| 331- | 33.3 | 56.0 | 700 | 2.7 | 10.3 | 5.4 |
| 1075(2) | | | | | | |
| | 23.6 | 53.9 | 1000 | 2.4 | 10.6 | 0.7 |
| | 17.0 | 53.2 | 1400 | 2.1 | 10.9 | 0.5 |
| 331- | 27.7 | 54.8 | 800 | 2.6 | 10.4 | 1.7 |
| 1093(1) | | | | | | |
| | 20.7 | 53.3 | 1100 | 2.3 | 10.7 | 0.4 |
| | 17.7 | 53.1 | 1400 | 2.2 | 10.8 | 0.5 |
| 331- | 25.7 | 54.7 | 800 | 2.6 | 10.4 | 1.0 |
| 1093(2) | | | | | | |
| | 17.9 | 53.6 | 1100 | 2.3 | 10.7 | 0.4 |
| | 15.8 | 52.3 | 1400 | 2.0 | 11.0 | trace |
| 331- | 25.8 | 56.2 | 705 | 2.8 | 10.2 | 1.3 |
| 1118(1) | | | | | | |
| | 18.7 | 56.0 | 1000 | 2.6 | 10.4 | 0.4 |
| | 14.3 | 54.7 | 1400 | 2.2 | 10.8 | 0.1 |
| 331- | 25.1 | 56.0 | 800 | 2.8 | 10.2 | 1.3 |
| 1118(2) | | | | | | |
| | 20.7 | 55.0 | 1000 | 2.5 | 10.5 | 0.4 |
| | 15.4 | 54.3 | 1400 | 2.3 | 10.7 | 0.1 |
| 331- | 25.8 | 55.3 | 800 | 2.5 | 10.5 | 1.6 |
| 1122(1) | | | | | | |
| | 18.7 | 53.7 | 1100 | 2.2 | 10.8 | 0.1 |
| | 14.6 | 53.3 | 1400 | 2.1 | 10.9 | 0.1 |
| 331- | 23.2 | 55.8 | 800 | 2.8 | 10.2 | 1.1 |
| 1126(1) | | | | | | |
| | 18.1 | 54.4 | 1100 | 2.5 | 10.5 | 0.1 |
| | 14.7 | 54.1 | 1400 | 2.3 | 10.7 | trace |
| 331- | 38.6 | 54.7 | 800 | 2.1 | 10.9 | 5.4 |
| 1136(1) | | | | | | |
| | 25.7 | 52.6 | 1100 | 1.9 | 12.1 | 1.7 |
| | 20.7 | 51.6 | 1400 | 1.8 | 12.2 | 1.1 |
| | 18.0 | 51.4 | 1634 | 1.7 | 11.3 | na |
| 331- | 24.1 | 54.9 | 800 | 2.9 | 10.1 | 0.5 |
| 1162(3) | | | | | | |
| | 17.1 | 53.4 | 1100 | 2.6 | 10.4 | trace |
| | 14.0 | 52.8 | 1400 | 2.4 | 10.6 | trace |
| 331- | 24.3 | 56.1 | 800 | 2.7 | 10.3 | 0.6 |
| 1186(3) | | | | | | |
| | 17.3 | 54.4 | 1100 | 2.4 | 10.6 | trace |
| | 14.2 | 54.4 | 1400 | 2.2 | 10.8 | 0.1 |
| 53- | 21.5 | 56.0 | 800 | 2.6 | 10.4 | 0.8 |
| 242(1) | | | | | | |
| | 16.9 | 54.5 | 1100 | 2.3 | 10.7 | 0.2 |
| | 14.1 | 54.0 | 1400 | 2.1 | 10.9 | trace |
| 53- | 23.0 | 56.5 | 800 | 2.7 | 10.3 | 2.7 |
| 242(2) | | | | | | |
| | 16.9 | 55.5 | 1100 | 2.5 | 10.5 | 1.0 |
| | 16.4 | 55.1 | 1400 | 2.3 | 10.7 | 2.4 |
| 53- | 23.3 | 55.9 | 800 | 2.7 | 10.3 | 1.4 |
| 246(1) | | | | | | |
| | 16.4 | 54.8 | 1100 | 2.4 | 10.6 | 0.2 |
| | 14.2 | 54.0 | 1400 | 2.2 | 10.8 | trace |
| 53- | 23.1 | 56.6 | 800 | 2.7 | 10.3 | 1.0 |
| 246(2) | | | | | | |
| | 17.4 | 56.1 | 1100 | 2.5 | 10.5 | 0.9 |
| | 12.8 | 55.2 | 1400 | 2.4 | 10.6 | trace |
| 93- | 22.6 | 58.0 | 800 | 2.8 | 10.2 | 2.4 |
| 968(1) | | | | | | |
| | 16.7 | 56.6 | 1100 | 2.6 | 10.4 | 0.2 |
| | 14.2 | 55.5 | 1400 | 2.2 | 10.8 | trace |
| 93- | 18.8 | 58.5 | 800 | 3.1 | 9.9 | 0.9 |
| 968(2) | | | | | | |
| | 13.2 | 57.4 | 1100 | 2.8 | 10.2 | 0.1 |
| | 11.9 | 56.1 | 1400 | 2.5 | 10.5 | trace |

TABLE X
Kraft pulping data for harvested stems (Kappa 17)

| | H-Factor | Unscreened Yield (%) | % EA Consumed |
|---|---|---|---|
| 14-129 | 1230 | 54.4 | 10.3 |
| | 1436 | 53.5 | 10.9 |
| ? 93-968 | 1110 | 56.5 | 10.5 |
| | 883 | 58.0 | 10.0 |
| ? 53-242 | 1092 | 54.7 | 10.7 |
| | 1211 | 55.4 | 10.6 |
| ? 53-246 | 1112 | 54.7 | 10.6 |
| | 1088 | 55.9 | 10.5 |
| 331-1059 | 1213 | 54.4 | 10.8 |
| | 1190 | 54.0 | 10.9 |
| 331-1061 | 1448 | 52.9 | 11.0 |
| | 1200 | 53.9 | 10.8 |
| | 1225 | 54.0 | 10.8 |
| 331-1062 | 1219 | 53.3 | 10.8 |
| | 1207 | 52.9 | 10.8 |
| 331-1075 | 1401 | 53.0 | 10.9 |
| 331-1093 | 1443 | 52.8 | 10.9 |
| | 1236 | 52.9 | 10.8 |
| ?331-1118 | 1135 | 55.3 | 10.6 |
| | 1251 | 54.5 | 10.6 |
| 331-1122 | 1206 | 53.6 | 10.8 |
| 331-1126 | 1177 | 54.4 | 10.5 |
| 331-1136 | 1684 | 51.1 | 11.3 |
| 331-1162 | 1132 | 53.4 | 10.4 |
| ?331-1186 | 1146 | 54.7 | 10.6 |

Bold = top yielding clones

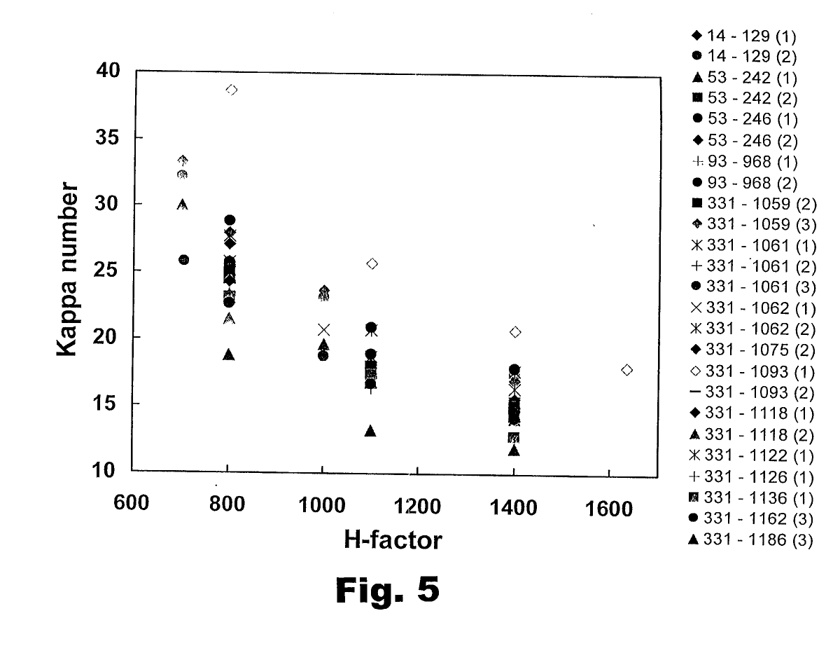

FIG. 5 shows the relationship between H-factor and Kappa number for the pulped stems. In FIG. 4, population parents 93-968 and 14-129 form the boundaries of the variability seen in kappa number at each H-factor value. It is clear that, as was the case for aspen, the variation in H-factor required to achieve a given Kappa number is substantial. For example, to achieve Kappa 17, clone 331-1136 requires approximately 1650H-factor whereas clone 93-968 requires only 1000H-factor (a 40% reduction).

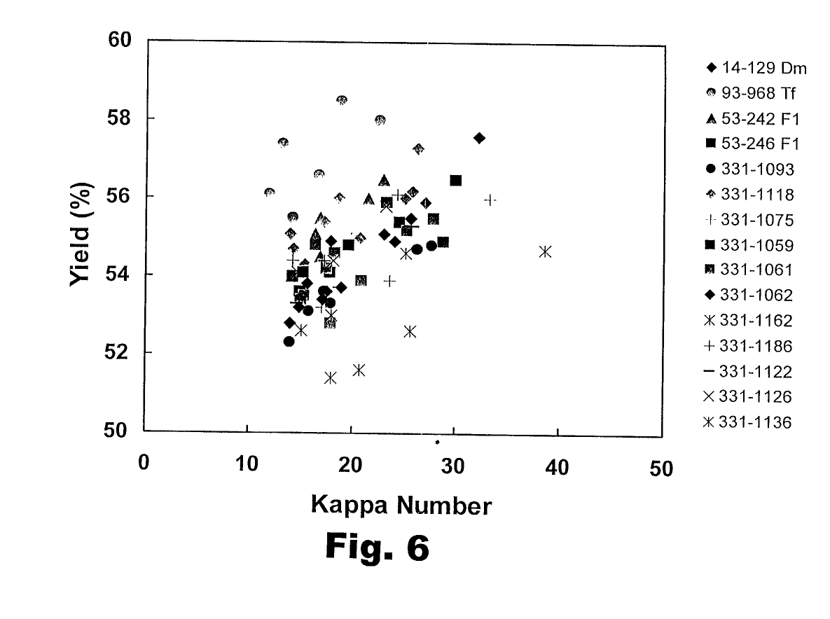

The particular difficulty in pulping clone 331-1136 indicated here may be a function of this clone's high level of calcium accumulation (see below), particularly as this clone's lignin content is not unusually high (24.56% in a population range of 22.93-25.75%, see Table IV. Also like aspen, the swings in yield at a given unbleached kappa number are substantial. All the exploratory kraft pulping data are presented in Table X herewith. At kappa 17 the yield from clone 331-1136 was approximately 51%. This may be an outlier point (excess compression wood due to plantation location, etc.). The lower limit of pulp yield is probably better represented by clones 331-1093 and 331-1062 whereas clone 93-968 exhibits a 57% pulp yield (FIG. 6). In FIG. 6, parent 93-968 (pure P. trichocarpa) forms a distinct envelope whereas the remainder of the clones examined resemble parent 14-129 (P. deltoides). Superior clones are highlighted in Table X. The relationship between ease of pulping and pulp yield is evident (Pearson correlation of −0.828, p=0.000).

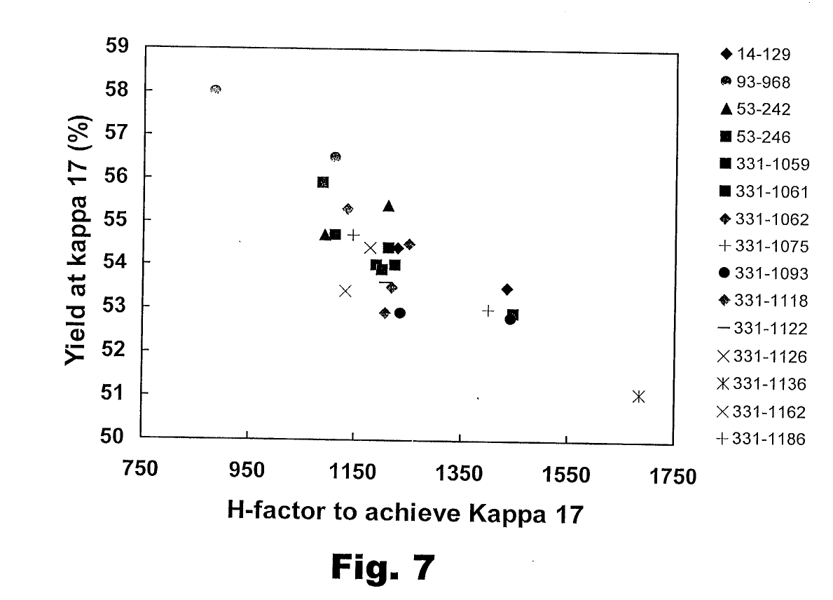

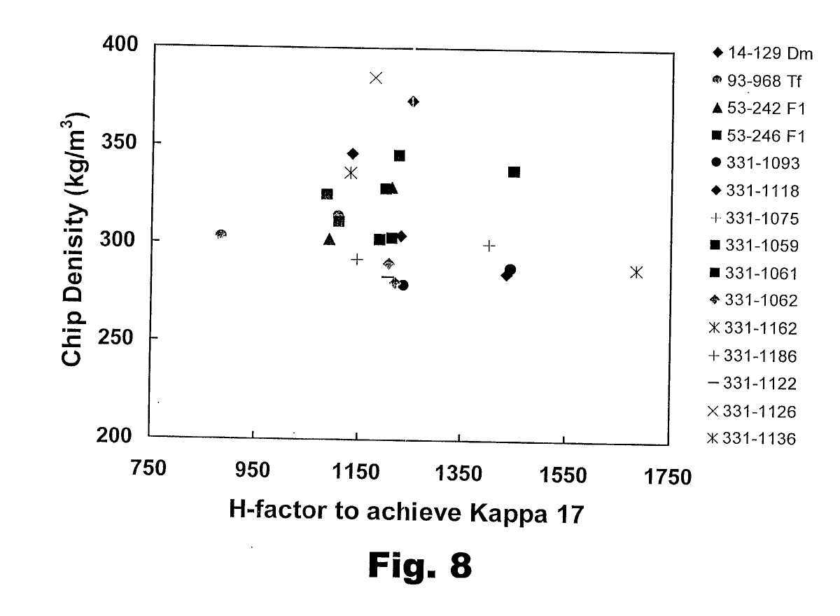

However it should be noted that the variability in yield at a given H-factor is high as evidenced by the relatively poor R² of 0.69, shown in FIG. 7. In FIG. 7, it can be seen that the Parental clones represent the extremes, (clonal lignin content 25.75-22.93%) 331-1162 has the lowest lignin content but gives low pulp yield and average pulping rate, therefore lignin content is not a reliable indicator of pulpability. These results confirm the necessity to pilot pulp clones for proper evaluation of properties. Further, the H-factor required to achieve kappa 17 has been evaluated against the chip density in FIG. 8. It is clear that in addition to lignin content wood density cannot be used to predict ease of kraft pulping (Pearson coefficient −0.194, p=1.000).

Table XI presents the fiber properties data obtained for the pulped clones at Kappa 17. The top three ranked clones in terms of high length and low coarseness are indicated in bold.

TABLE XI
Whole stem pulp fibre properties data

| | LW Fiber Length | Coarseness |
|---|---|---|
| | (mm) | (mg/m) |
| 14-129 | 0.65 | 0.103 |
| | 0.69 | 0.115 |
| 93-968 | 0.66 | 0.097 |
| | 0.76 | 0.113 |
| 53-242 | 0.69 | 0.099 |
| | 0.76 | 0.109 |
| 53-246 | 0.73 | 0.105 |
| | 0.74 | 0.103 |
| 331-1059 | 0.67 | 0.087 |
| | 0.65 | 0.092 |
| 331-1061 | 0.68 | 0.097 |
| | 0.64 | 0.094 |
| | 0.71 | 0.101 |
| ?331-1062 | ?0.80 | ?0.121 |
| | 0.82 | 0.121 |
| 331-1075 | 0.69 | 0.097 |
| 331-1093 | 0.53 | 0.083 |
| | 0.57 | 0.083 |
| 331-1118 | 0.78 | 0.105 |
| | 0.61 | 0.101 |
| ?331-1122 | ?0.79 | ?0.122 |
| 331-1126 | 0.79 | 0.102 |
| 331-1136 | 0.46 | 0.117 |
| ?331-1162 | ?0.80 | ?0.121 |
| 331-1186 | 0.68 | 0.099 |

Bold = top 3 coarse fiberd clones
Italics = top 3 fine fiberd clones

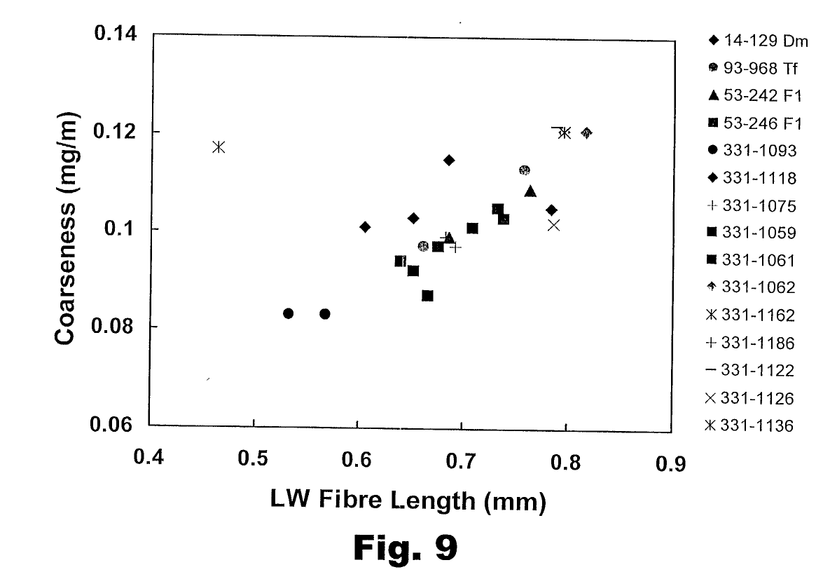

A positive correlation (Pearson coefficient 0.543, p=0.105) can be seen between the fiber length and coarseness data which mirrors that seen for the 7^{th} year ring data and the situation seen in aspen populations (FIG. 9). In FIG. 9, the positive correlation seen here is in contrast to that seen for aspen clones but supports the data obtained for the 7^{th} year growth ring from each hybrid poplar in the previous study. If the outlier point for clone 331-1136 is omitted from the analysis, the correlation becomes much more significant (Pearson coefficient 0.834, p=0.000).

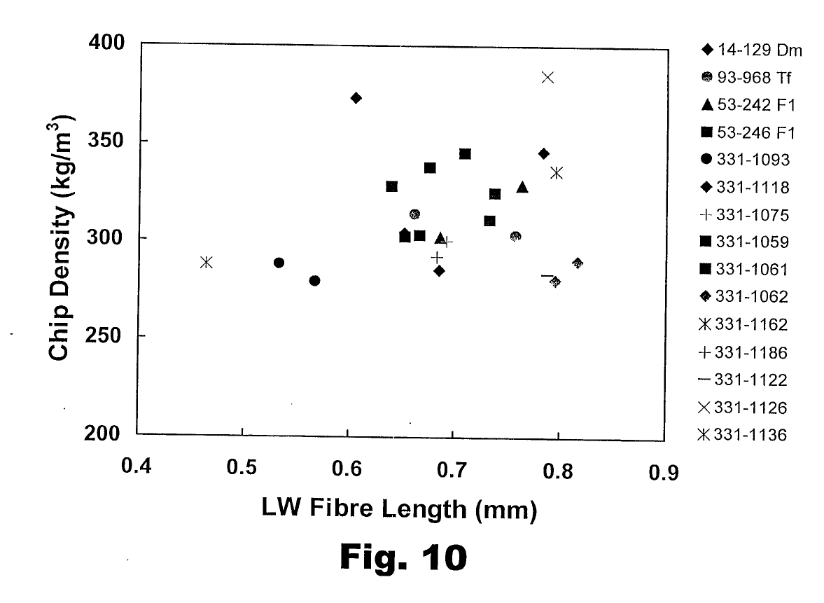

The length-weighted fiber length data were also correlated to chip density values, as shown in FIG. 10. Not unexpectedly, and bearing in mind the fiber length: coarseness relationship, the relationship is poor (Pearson coefficient 0.228, p=1.000) even if outlier points are excluded.

Pulp yield data at kappa 17, were used in a Mapmaker-QTL 1.1 analysis which revealed the presence of a single, low significance QTL for this property—Table XII. The pilot-scale pulping of further clones will likely enhance the statistical significance of the detection of this QTL. Significantly, the QTL kraft pulp yield (the most important trait from an industrial production point of view) correlate with a higher significance QTL for maceration yield but does not coincide with the lignin QTL (Table V).

TABLE XII
Low significance QTL detected for Kraft pulp yield

| Trait | Marker/Linkage | LOD Score | Phen % | Length/cM | Weight | Dom. |
|---|---|---|---|---|---|---|
| Kraft pulp yield | P1027-P192/R | 2.52* | 72.7 | 0.0 | −1.8932 | 0.7270 |

H-factor to kappa 17 data from Table IX were also used in a Mapmaker QTL1.1 analysis. However, no significant QTLs were observed which confirms that, not surprisingly, lignin content is not the single controlling factor in kraft pulping of hybrid poplar. There may be concern that this observation does not seem to relate to measurable physical properties. However, issues such as pulping liquor diffusion are also known to be a major contributor to ease of kraft pulping.

2. Kraft Pulp Properties

Kraft Pulp Strengths

The strength of hardwood pulps is becoming an increasingly important parameter given the economic impetus for lighter weight products which retain strength and optical properties and to reduce the amount of expensive softwood Kraft pulp required for many paper grades. Four point PFI mill beater curves were developed for each of the clonal pulps and the results of all tests are presented in Table XIII.

TABLE XIII
Hybrid Poplar Kraft Pulp and Optical Property data

| | 14-129 (1) | | | | 14-129 (2) | | | | 331-1059 (2) | | | |
|---|---|---|---|---|---|---|---|---|---|---|---|---|
| PFI Revolutions | 0 | 1000 | 3000 | 6000 | 0 | 1000 | 3000 | 6000 | 0 | 1000 | 3000 | 6000 |
| Screened Csf (mL) | 499 | 480 | 414 | 361 | 533 | 479 | 423 | 353 | 453 | 435 | 362 | 322 |
| Apparent Density (kg/m³) | 636 | 703 | 739 | 754 | 618 | 705 | 740 | 767 | 666 | 775 | 784 | 784 |
| Burst Index (kPa · m²/g) | 4.7 | 6.2 | 7.0 | 7.6 | 4.2 | 5.8 | 6.6 | 7.1 | 6.1 | 7.9 | 8.8 | 9.5 |
| Breaking length (km) | 8.7 | 9.3 | 10.6 | 10.5 | 8.2 | 9.1 | 9.7 | 10.1 | 9.3 | 10.6 | 11.3 | 11.6 |
| Tensile Index (N · m/g) | 85.1 | 90.9 | 104.1 | 103.4 | 79.9 | 89.2 | 95.1 | 99.2 | 90.9 | 104.0 | 111.1 | 113.9 |
| Stretch (%) | 1.58 | 2.58 | 3.44 | 3.68 | 1.60 | 2.71 | 2.97 | 3.55 | 3.11 | 4.46 | 5.01 | 5.26 |
| Tear Index (mN · m²/g) (1 | 6.0 | 7.2 | 7.5 | 7.9 | 5.6 | 6.6 | 7.1 | 6.7 | 8.3 | 9.4 | 9.0 | 9.0 |
| Ply) | | | | | | | | | | | | |
| Tear Index (mN · m²/g) (4 | 7.2 | 7.6 | 7.6 | 7.5 | 7.7 | 7.4 | 7.6 | 7.4 | 8.7 | 9.1 | 9.0 | 8.6 |
| Ply) | | | | | | | | | | | | |
| Zero Span Breaking Length | 15.9 | 15.1 | 15.8 | 15.5 | 15.3 | 15.6 | 16.3 | 16.0 | 14.0 | 13.4 | 13.4 | 12.8 |
| (km) | | | | | | | | | | | | |
| Air Resistance (Gurley) | 65.0 | 121.5 | 206.8 | 372.4 | 42.0 | 85.4 | 133.4 | 292.8 | 130.6 | 249.6 | 476.2 | 862.1 |
| (sec/100 mL) | | | | | | | | | | | | |
| Sheffield Roughness | 89 | 52 | 40 | 27 | 107 | 68 | 52 | 33 | 61 | 31 | 22 | 17 |
| (mL/min) | | | | | | | | | | | | |
| Brightness | 37 | | | | 37 | | | | 38 | | | |
| Opacity (%) | 96.0 | 95.9 | 94.4 | 93.0 | 97.3 | 96.1 | 93.9 | 92.3 | 96.8 | 95.2 | 94.0 | 92.1 |
| Scattering Coefficient | 311 | 289 | 258 | 229 | 338 | 286 | 242 | 211 | 327 | 266 | 221 | 197 |
| (cm²/g) | | | | | | | | | | | | |
| | 331-1059 (3) | | | | 331-1061 (1) | | | | 331-1061 (2) | | | |
| PFI Revolutions | 0 | 1000 | 3000 | 6000 | 0 | 1000 | 3000 | 6000 | 0 | 1000 | 3000 | 6000 |
| Screened Csf (mL) | 486 | 454 | 372 | 339 | 524 | 478 | 395 | 346 | 524 | 478 | 395 | 346 |
| Apparent Density (kg/m³) | 663 | 717 | 757 | 765 | 648 | 721 | 755 | 786 | 682 | 734 | 793 | 807 |
| Burst Index (kPa · m²/g) | 6.1 | 7.5 | 8.2 | 8.7 | 4.9 | 6.1 | 6.9 | 7.3 | 4.9 | 6.5 | 7.6 | 8.1 |
| Breaking length (km) | 9.1 | 9.6 | 9.9 | 10.7 | 8.2 | 9.2 | 9.9 | 10.8 | 8.3 | 9.2 | 10.1 | 10.8 |
| Tensile Index (N · m/g) | 88.8 | 93.7 | 97.3 | 105.0 | 80.7 | 90.3 | 97.4 | 106.1 | 81.2 | 90.3 | 99.0 | 105.8 |
| Stretch (%) | 2.78 | 3.91 | 4.77 | 7.95 | 1.96 | 2.90 | 3.45 | 4.25 | 1.99 | 3.35 | 3.73 | 4.45 |
| Tear Index (mN · m²/g) (1 | 7.5 | 8.9 | 8.9 | 9.0 | 7.9 | 8.8 | 8.4 | 8.7 | 6.2 | 8.0 | 7.8 | 8.0 |
| Ply) | | | | | | | | | | | | |
| Tear Index (mN · m²/g) (4 | 8.2 | 8.3 | 8.4 | 8.4 | 8.2 | 8.2 | 8.5 | 8.5 | 7.9 | 8.3 | 8.6 | 8.1 |
| Ply) | | | | | | | | | | | | |
| Zero Span Breaking Length | 14.7 | 13.9 | 14.0 | 13.7 | 16.3 | 15.2 | 15.0 | 13.7 | 15.8 | 16.1 | 16.2 | 16.0 |
| (km) | | | | | | | | | | | | |
| Air Resistance (Gurley) | 119.8 | 177.4 | 325.0 | 537.0 | 75.8 | 147.3 | 219.6 | 449.7 | 55.1 | 101.1 | 201.0 | 359.9 |
| (sec/100 mL) | | | | | | | | | | | | |
| Sheffield Roughness | 62 | 40 | 30 | 23 | 79 | 53 | 41 | 27 | 87 | 59 | 37 | 26 |
| (mL/min) | | | | | | | | | | | | |
| Brightness | 37 | | | | 35 | | | | 38 | | | |
| Opacity (%) | 96.5 | 95.0 | 93.0 | 91.5 | 96.4 | 93.9 | 93.0 | 91.9 | 95.4 | 94.5 | 93.1 | 91.4 |
| Scattering Coefficient | 323 | 253 | 214 | 193 | 298 | 243 | 222 | 200 | 305 | 269 | 232 | 212 |
| (cm²/g) | | | | | | | | | | | | |
| | 331-1061 (3) | | | | 331-1062 (1) | | | | 331-1062 (2) | | | |
| PFI Revolutions | 0 | 1000 | 3000 | 6000 | 0 | 1000 | 3000 | 6000 | 0 | 1000 | 3000 | 6000 |
| Screened Csf (mL) | 552 | 492 | 420 | 353 | 554 | 536 | 469 | 412 | 561 | 527 | 466 | 397 |
| Apparent Density (kg/m³) | 625 | 705 | 736 | 748 | 642 | 716 | 745 | 775 | 619 | 702 | 735 | 757 |
| Burst Index (kPa · m²/g) | 4.3 | 6.1 | 7.1 | 7.4 | 4.9 | 6.1 | 7.1 | 7.6 | 4.9 | 6.2 | 6.7 | 7.6 |
| Breaking length (km) | 7.7 | 8.8 | 9.0 | 10.5 | 9.2 | 9.2 | 10.1 | 10.8 | 8.5 | 9.3 | 10.1 | 10.4 |
| Tensile Index (N · m/g) | 75.9 | 86.0 | 88.7 | 102.6 | 89.8 | 90.6 | 98.9 | 106.0 | 83.3 | 90.9 | 98.9 | 101.7 |
| Stretch (%) | 1.69 | 2.91 | 3.10 | 4.13 | 1.98 | 2.69 | 3.44 | 3.88 | 1.66 | 2.83 | 3.39 | 3.45 |
| Tear Index (mN · m²/g) (1 | 6.2 | 9.0 | 8.6 | 9.2 | 8.6 | 8.7 | 8.6 | 8.5 | 7.2 | 7.2 | 7.8 | 8.4 |
| Ply) | | | | | | | | | | | | |
| Tear Index (mN · m²/g) (4 | 8.2 | 9.3 | 9.0 | 9.0 | 8.9 | 8.7 | 8.5 | 8.2 | 8.7 | 8.9 | 8.5 | 8.2 |
| Ply) | | | | | | | | | | | | |
| Zero Span Breaking Length | 15.9 | 16.3 | 15.2 | 14.2 | 17.6 | 17.0 | 15.7 | 15.8 | 15.2 | 15.0 | 15.0 | 15.3 |
| (km) | | | | | | | | | | | | |
| Air Resistance (Gurley) | 28.4 | 74.8 | 140.6 | 234.1 | 72.5 | 148.7 | 279.1 | 562.1 | 51.7 | 115.6 | 210.5 | 412.4 |
| (sec/100 mL) | | | | | | | | | | | | |
| Sheffield Roughness | 115 | 76 | 55 | 39 | 87 | 55 | 38 | 27 | 109 | 68 | 43 | 28 |
| (mL/min) | | | | | | | | | | | | |
| Brightness | 37 | | | | 36 | | | | 37 | | | |
| Opacity (%) | 95.2 | 93.4 | 92.2 | 90.9 | 94.9 | 93.0 | 91.6 | 89.3 | 95.2 | 92.5 | 90.8 | 89.2 |
| Scattering Coefficient | 304 | 254 | 229 | 204 | 268 | 221 | 193 | 167 | 286 | 233 | 201 | 179 |
| (cm²/g) | | | | | | | | | | | | |
| | 331-1075 (2) | | | | 331-1093 (1) | | | | 331-1093 (2) | | | |
| PFI Revolutions | 0 | 1000 | 3000 | 6000 | 0 | 1000 | 3000 | 6000 | 0 | 1000 | 3000 | 6000 |
| Screened Csf (mL) | 483 | 451 | 375 | 328 | 405 | 393 | 336 | 298 | 425 | 403 | 354 | 294 |
| Apparent Density (kg/m³) | 701 | 781 | 813 | 816 | 734 | 807 | 789 | 861 | 679 | 696 | 742 | 749 |
| Burst Index (kPa · m²/g) | 6.2 | 7.5 | 8.0 | 8.5 | 7.0 | 8.0 | 8.7 | 9.4 | 6.3 | 7.7 | 8.1 | 8.8 |
| Breaking length (km) | 9.8 | 10.3 | 10.9 | 11.5 | 11.3 | 11.6 | 12.2 | 12.1 | 10.5 | 10.2 | 10.7 | 11.5 |
| Tensile Index (N · m/g) | 96.2 | 101.3 | 106.5 | 113.1 | 111.2 | 113.5 | 119.2 | 119.0 | 102.6 | 99.8 | 104.8 | 113.2 |
| Stretch (%) | 2.58 | 3.41 | 3.97 | 4.73 | 2.53 | 3.77 | 4.35 | 4.61 | 2.71 | 3.42 | 3.89 | 4.80 |
| Tear Index (mN · m²/g) (1 | 8.1 | 7.9 | 8.1 | 7.8 | 6.7 | 7.5 | 7.7 | 7.8 | 8.6 | 7.8 | 8.4 | 8.4 |
| Ply) | | | | | | | | | | | | |
| Tear Index (mN · m²/g) (4 | 9.0 | 9.1 | 8.5 | 8.3 | 8.3 | 7.9 | 8.0 | 7.4 | 8.2 | 8.0 | 8.1 | 8.0 |
| Ply) | | | | | | | | | | | | |
| Zero Span Breaking Length | 16.3 | 14.7 | 14.3 | 13.2 | 15.0 | 14.7 | 14.3 | 13.8 | 15.7 | 15.1 | 14.5 | 14.1 |
| (km) | | | | | | | | | | | | |
| Air Resistance (Gurley) | 105.9 | 281.4 | 510.0 | 1152.7 | 274.7 | 409.6 | 719.4 | 1351.2 | 202.8 | 527.0 | 802.0 | 1378.1 |
| (sec/100 mL) | | | | | | | | | | | | |
| Sheffield Roughness | 61 | 34 | 22 | 15 | 37 | 25 | 17 | 13 | 46 | 25 | 16 | 10 |
| (mL /min) | | | | | | | | | | | | |
| Brightness | 35 | | | | 38 | | | | 38 | | | |
| Opacity (%) | 95.3 | 93.0 | 91.8 | 89.0 | 96.1 | 94.2 | 92.4 | 91.0 | 96.1 | 94.0 | 92.8 | 90.3 |
| Scattering Coefficient | 287 | 224 | 201 | 169 | 318 | 260 | 230 | 204 | 323 | 260 | 233 | 203 |
| (cm²/g) | | | | | | | | | | | | |
| | 331-1118 (1) | | | | 331-1118 (2) | | | | 331-1122 (1) | | | |
| PFI Revolutions | 0 | 1000 | 3000 | 6000 | 0 | 1000 | 3000 | 6000 | 0 | 1000 | 3000 | 6000 |
| Screened Csf (mL) | 532 | 487 | 401 | 344 | 573 | 538 | 499 | 443 | 553 | 493 | 453 | 406 |
| Apparent Density (kg/m³) | 585 | 628 | 692 | 708 | 613 | 701 | 734 | 722 | 660 | 734 | 737 | 780 |
| Burst Index (kPa · m²/g) | 4.3 | 5.8 | 6.8 | 7.5 | 4.0 | 6.1 | 6.8 | 7.9 | 4.6 | 6.1 | 6.9 | 7.4 |
| Breaking length (km) | 6.9 | 8.4 | 9.5 | 9.5 | 7.0 | 8.4 | 9.1 | 10.8 | 7.8 | 9.5 | 9.8 | 10.2 |
| Tensile Index (N · m/g) | 67.2 | 82.1 | 92.8 | 93.5 | 68.4 | 82.5 | 89.6 | 106.1 | 76.7 | 92.8 | 95.7 | 99.6 |
| Stretch (%) | 2.42 | 3.86 | 4.67 | 4.74 | 2.01 | 3.22 | 4.24 | 4.80 | 1.68 | 3.07 | 3.52 | 3.82 |
| Tear Index (mN · m²/g) (1 | 7.1 | 8.6 | 8.6 | 9.1 | 6.6 | 8.6 | 9.5 | 10.5 | 7.3 | 8.8 | 8.7 | 8.4 |
| Ply) | | | | | | | | | | | | |
| Tear Index (mN · m²/g) (4 | 8.6 | 8.4 | 8.7 | 8.8 | 8.6 | 9.4 | 9.5 | 10.1 | 8.6 | 9.0 | 8.4 | 8.3 |
| Ply) | | | | | | | | | | | | |
| Zero Span Breaking Length | 13.1 | 12.7 | 13.3 | 13.4 | 14.1 | 14.2 | 14.6 | 15.2 | 14.4 | 14.3 | 14.0 | 14.0 |
| (km) | | | | | | | | | | | | |
| Air Resistance (Gurley) | 26.3 | 65.7 | 112.5 | 209.0 | 13.3 | 28.8 | 50.1 | 101.7 | 57.4 | 104.0 | 244.3 | 312.5 |
| (sec/100 mL) | | | | | | | | | | | | |
| Sheffield Roughness | 137 | 88 | 70 | 50 | 142 | 103 | 99 | 65 | 98 | 73 | 50 | 40 |
| (mL/min) | | | | | | | | | | | | |
| Brightness | 39 | | | | 38 | | | | 36 | | | |
| Opacity (%) | 97.7 | 96.0 | 95.0 | 93.6 | 96.7 | 94.2 | 92.0 | 91.1 | 94.9 | 92.3 | 89.8 | 89.2 |
| Scattering Coefficient | 363 | 290 | 252 | 221 | 345 | 264 | 225 | 197 | 268 | 216 | 185 | 169 |
| (cm²/g) | | | | | | | | | | | | |
| | 331-1126 (1) | | | | 331-1136 (1) | | | | 331-1162 (3) | | | |
| PFI Revolutions | 0 | 1000 | 3000 | 6000 | 0 | 1000 | 3000 | 6000 | 0 | 1000 | 3000 | 6000 |
| Screened Csf (mL) | 577 | 530 | 476 | 422 | 415 | 409 | 373 | 365 | 497 | 457 | 400 | 346 |
| Apparent Density (kg/m³) | 609 | 695 | 723 | 742 | 620 | 652 | 690 | 678 | 648 | 707 | 751 | 760 |
| Burst Index (kPa · m²/g) | 3.4 | 5.4 | 6.5 | 7.2 | 5.9 | 6.9 | 7.4 | 7.6 | 5.2 | 6.8 | 8.1 | 8.5 |
| Breaking length (km) | 6.7 | 8.0 | 8.9 | 10.1 | 8.8 | 9.3 | 10.0 | 10.6 | 9.5 | 10.3 | 11.2 | 11.5 |
| Tensile Index (N · m/g) | 65.6 | 78.5 | 86.9 | 99.0 | 86.2 | 91.2 | 97.6 | 104.0 | 93.4 | 101.3 | 109.9 | 112.3 |
| Stretch (%) | 1.47 | 2.58 | 3.12 | 3.83 | 3.32 | 3.75 | 4.51 | 5.40 | 2.24 | 3.25 | 3.90 | 4.38 |
| Tear Index (mN · m²/g) (1 | 6.0 | 8.5 | 8.2 | 8.5 | 7.8 | 8.5 | 8.3 | 8.3 | 8.5 | 7.8 | 8.3 | 8.3 |
| Ply) | | | | | | | | | | | | |
| Tear Index (mN · m²/g) (4 | 8.3 | 9.2 | 9.1 | 8.7 | 8.1 | 8.0 | 7.5 | 7.7 | 9.8 | 9.7 | 9.7 | 9.7 |
| Ply) | | | | | | | | | | | | |
| Zero Span Breaking Length | 14.9 | 14.8 | 14.7 | 14.7 | 14.2 | 14.7 | 12.9 | 12.4 | 16.8 | 15.5 | 16.4 | 16.7 |
| (km) | | | | | | | | | | | | |
| Air Resistance (Gurley) | 10.6 | 21.3 | 41.7 | 65.0 | 563.9 | 1128.1 | >30 | >30 | 39.0 | 79.3 | 152.3 | 223.8 |
| (sec/100 mL) | | | | | | | min | min | | | | |
| Sheffield Roughness | 161 | 111 | 92 | 76 | 65 | 32 | 20 | 17 | 100 | 68 | 50 | 41 |
| (mL/min) | | | | | | | | | | | | |
| Brightness | 38 | | | | 33 | | | | 39 | | | |
| Opacity (%) | 96.0 | 94.6 | 92.8 | 92.0 | 95.8 | 95.0 | 93.2 | 91.2 | 96.7 | 95.5 | 94.2 | 93.6 |
| Scattering Coefficient | 323 | 273 | 238 | 219 | 269 | 233 | 195 | 165 | 344 | 292 | 251 | 234 |
| (cm²/g) | | | | | | | | | | | | |
| | 331-1186 (3) | | | | 53-242 (1) | | | | 53-242 (2) | | | |
| PFI Revolutions | 0 | 1000 | 3000 | 6000 | 0 | 1000 | 3000 | 6000 | 0 | 1000 | 3000 | 6000 |
| Screened Csf (mL) | 489 | 481 | 418 | 357 | 569 | 510 | 440 | 389 | 513 | 472 | 405 | 350 |
| Apparent Density (kg/m³) | 673 | 716 | 759 | 770 | 631 | 691 | 723 | 741 | 640 | 722 | 779 | 785 |
| Burst Index (kPa · m²/g) | 6.0 | 7.3 | 8.5 | 8.9 | 4.6 | 6.5 | 7.2 | 7.7 | 5.6 | 7.0 | 8.0 | 8.5 |
| Breaking length (km) | 9.3 | 10.2 | 11.4 | 11.3 | 7.8 | 9.3 | 9.6 | 10.4 | 9.0 | 10.1 | 10.4 | 11.4 |
| Tensile Index (N · m/g) | 91.2 | 100.2 | 112.0 | 110.6 | 76.2 | 91.5 | 94.4 | 102.3 | 88.5 | 98.8 | 102.0 | 111.6 |
| Stretch (%) | 2.25 | 3.55 | 4.65 | 4.59 | 1.76 | 3.39 | 3.68 | 4.15 | 2.21 | 3.18 | 3.65 | 4.49 |
| Tear Index (mN · m²/g) (1 | 7.5 | 8.6 | 8.7 | 8.4 | 7.5 | 8.3 | 8.7 | 8.5 | 7.7 | 8.7 | 8.8 | 8.4 |
| Ply) | | | | | | | | | | | | |
| Tear Index (mN · m²/g) (4 | 8.5 | 8.7 | 8.2 | 8.5 | 8.4 | 8.6 | 8.6 | 8.7 | 7.8 | 7.7 | 7.5 | 7.6 |
| Ply) | | | | | | | | | | | | |
| Zero Span Breaking Length | 15.4 | 15.2 | 15.6 | 15.2 | 16.1 | 16.0 | 16.5 | 15.0 | 14.3 | 13.4 | 15.6 | 14.3 |
| (km) | | | | | | | | | | | | |
| Air Resistance (Gurley) | 79.9 | 166.7 | 294.7 | 538.4 | 32.1 | 80.8 | 148.0 | 271.4 | 72.7 | 136.8 | 243.2 | 402.1 |
| (sec/100 mL) | | | | | | | | | | | | |
| Sheffield Roughness | 74 | 45 | 36 | 26 | 106 | 71 | 54 | 35 | 81 | 55 | 35 | 28 |
| (mL/min) | | | | | | | | | | | | |
| Brightness | 38 | | | | 40 | | | | 39 | | | |
| Opacity (%) | 95.7 | 93.9 | 92.4 | 91.3 | 95.1 | 92.2 | 91.0 | 89.8 | 95.5 | 93.6 | 91.8 | 90.0 |
| Scattering Coefficient | 302 | 247 | 222 | 194 | 325 | 250 | 224 | 202 | 301 | 249 | 219 | 193 |
| (cm²/g) | | | | | | | | | | | | |
| | 53-246 (1) | | | | 53-246 (2) | | | | 93-968 (1) | | | |
| PFI Revolutions | 0 | 1000 | 3000 | 6000 | 0 | 1000 | 3000 | 6000 | 0 | 1000 | 3000 | 6000 |
| Screened Csf (mL) | 549 | 491 | 436 | 385 | 550 | 531 | 468 | 389 | 550 | 508 | 429 | 368 |
| Apparent Density (kg/m³) | 651 | 710 | 746 | 765 | 615 | 707 | 737 | 775 | 617 | 657 | 720 | 737 |
| Burst Index (kPa · m²/g) | 4.3 | 6.5 | 7.3 | 7.7 | 4.3 | 6.1 | 7.2 | 7.3 | 5.0 | 6.4 | 7.3 | 7.6 |
| Breaking length (km) | 8.1 | 9.1 | 10.0 | 10.0 | 7.4 | 8.9 | 9.1 | 10.0 | 9.0 | 8.9 | 10.3 | 10.5 |
| Tensile Index (N · m/g) | 79.7 | 89.2 | 98.3 | 98.5 | 72.6 | 87.3 | 89.0 | 98.5 | 87.8 | 87.6 | 100.9 | 102.6 |
| Stretch (%) | 2.08 | 3.54 | 4.15 | 4.28 | 2.00 | 3.59 | 3.78 | 4.76 | 2.11 | 2.97 | 3.80 | 4.11 |
| Tear Index (mN · m²/g) (1 | 7.0 | 8.2 | 8.0 | 8.6 | 7.1 | 7.8 | 8.5 | 8.1 | 8.2 | 8.5 | 8.7 | 8.0 |
| Ply) | | | | | | | | | | | | |
| Tear Index (mN · m²/g) (4 | 7.7 | 8.3 | 8.4 | 8.1 | 8.2 | 8.5 | 8.4 | 8.2 | 8.5 | 8.2 | 8.0 | 8.1 |
| Ply) | | | | | | | | | | | | |
| Zero Span Breaking Length | 15.5 | 14.5 | 14.7 | 15.4 | 14.9 | 14.6 | 14.0 | 15.3 | 16.2 | 15.0 | 15.6 | 14.9 |
| (km) | | | | | | | | | | | | |
| Air Resistance (Gurley) | 48.2 | 114.9 | 195.2 | 306.4 | 32.8 | 77.0 | 146.0 | 207.4 | 39.0 | 82.2 | 146.1 | 261.2 |
| (sec/100 mL) | | | | | | | | | | | | |
| Sheffield Roughness | 92 | 59 | 40 | 30 | 119 | 75 | 54 | 38 | 113 | 76 | 54 | 43 |
| (mL/min) | | | | | | | | | | | | |
| Brightness | 40 | | | | 40 | | | | 41 | | | |
| Opacity (%) | 95.8 | 93.9 | 92.5 | 90.3 | 96.0 | 94.8 | 91.9 | 91.3 | 95.4 | 93.6 | 92.0 | 91.0 |
| Scattering Coefficient | 341 | 272 | 235 | 211 | 347 | 287 | 240 | 226 | 333 | 282 | 248 | 228 |
| (cm²/g) | | | | | | | | | | | | |

| | 93-968 (2) | | | |
|---|---|---|---|---|
| PFI Revolutions | 0 | 1000 | 3000 | 6000 |
| Screened Csf (mL) | 468 | 455 | 409 | 340 |
| Apparent Density (kg/m³) | 555 | 642 | 679 | 690 |
| Burst Index (kPa · m²/g) | 4.5 | 6.0 | 6.9 | 7.5 |
| Breaking length (km) | 8.0 | 9.2 | 10.0 | 10.4 |
| Tensile Index (N · m/g) | 78.7 | 89.9 | 98.0 | 101.9 |
| Stretch (%) | 1.90 | 2.95 | 3.62 | 3.80 |
| Tear Index (mN · m²/g) (1 | 6.1 | 7.2 | 7.6 | 7.6 |
| Ply) | | | | |
| Tear Index (mN · m²/g) (4 | 6.9 | 7.2 | 7.1 | 7.1 |
| Ply) | | | | |
| Zero Span Breaking Length | 14.2 | 14.7 | 14.6 | 14.4 |
| (km) | | | | |
| Air Resistance (Gurley) | 51.3 | 81.1 | 117.7 | 190.4 |
| (sec/100 mL) | | | | |
| Sheffield Roughness | 131 | 91 | 63 | 50 |
| (mL/min) | | | | |
| Brightness | 39 | | | |
| Opacity (%) | 95.3 | 94.2 | 93.4 | 92.7 |
| Scattering Coefficient | 319 | 288 | 263 | 244 |
| (cm²/g) | | | | |

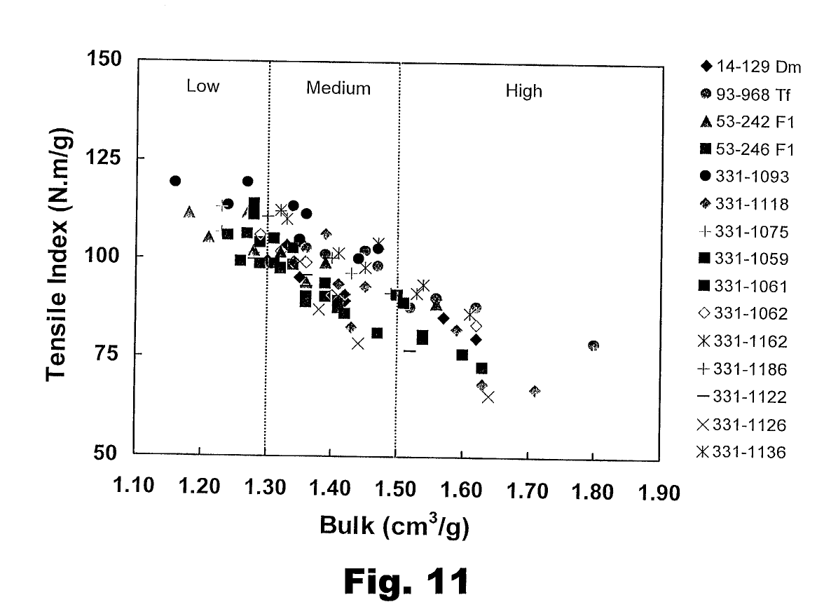

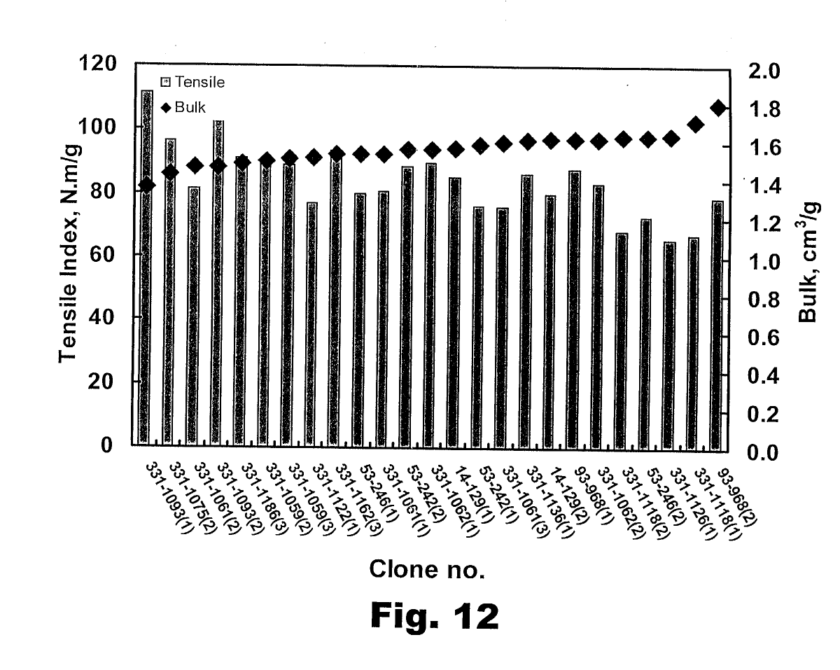

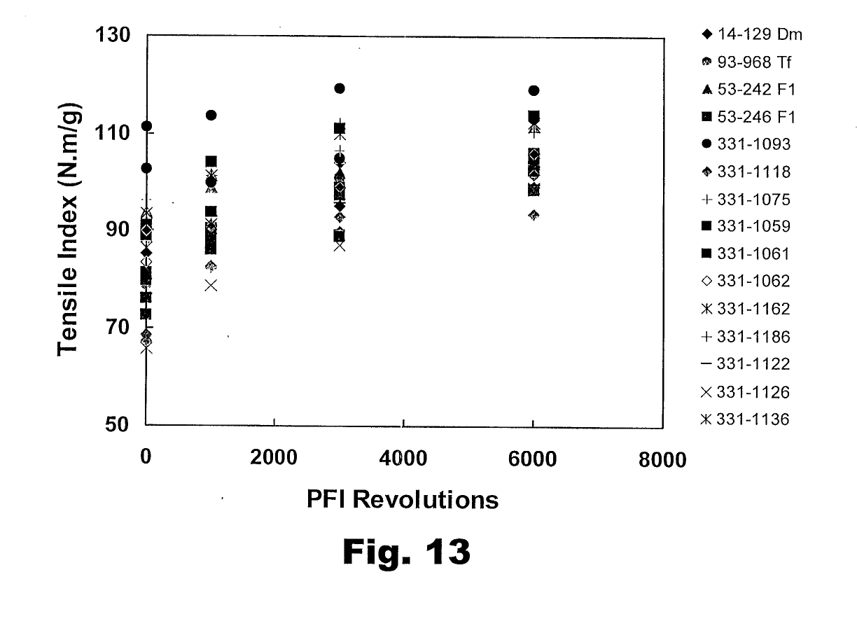

In a plot of tensile index vs. bulk, presented in FIG. 11, it can be seen that there is a strong negative correlation between the properties (Pearson coefficient −0.74, p=0.001). In FIG. 11, negative relationship confirms previous aspen data. Most clones show superior strength properties when compared to average values for Eucalyptus species (tensile index 70 N·m/g). More importantly, some clonal pulps (e.g. 331-1122, 1.26 cm³/g @ 100 N·m/g) are less bulky at given tensile strengths than are others [e.g. 331-1136, 1.45 cm³/g @ 100 N·m/g. (FIG. 12)] This was not predicted from the coarseness data in Table XI (331-1122, 0.122 mg/m vs 331-1136, 0.117 mg/m) and highlights the importance of carrying out pilot scale pulping trials. A coarseness cutoff of <0.1 mg/m is adequate for predicting low bulk/high tensile/fine fibers. It is worth nothing that for pulps prepared from eucalyptus species (the major competitor envisaged for Northern Populus plantation resources)—a tensile index value of 70 N·m/g is considered “standard”. Most of the hybrid poplar pulps examined in this study exceed that strength value even in an unbeaten state (FIG. 13). Additionally, the wide range of tensile indices suggest that there is wide variation in cell wall properties amongst the clones, a possibility which opens up potential multiple end-use applications for the pulps.

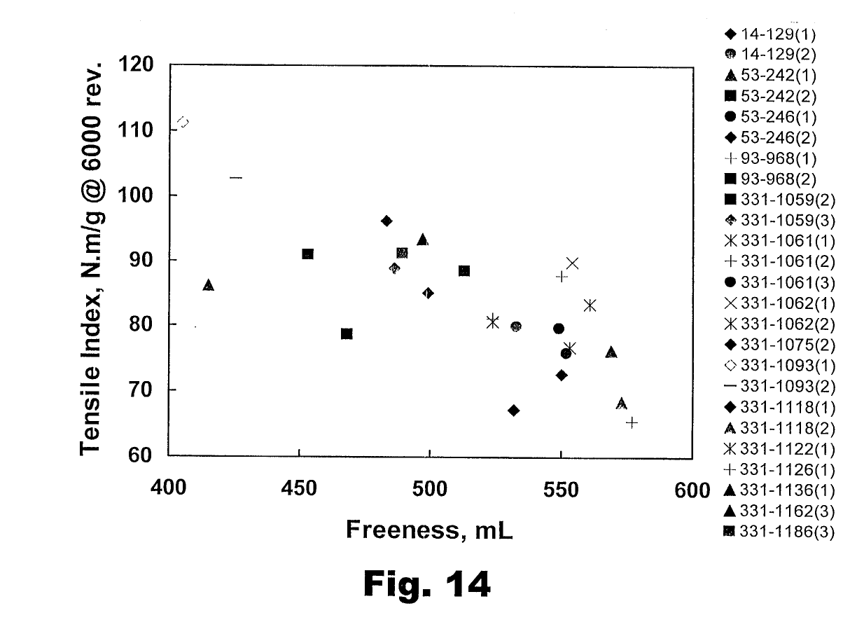

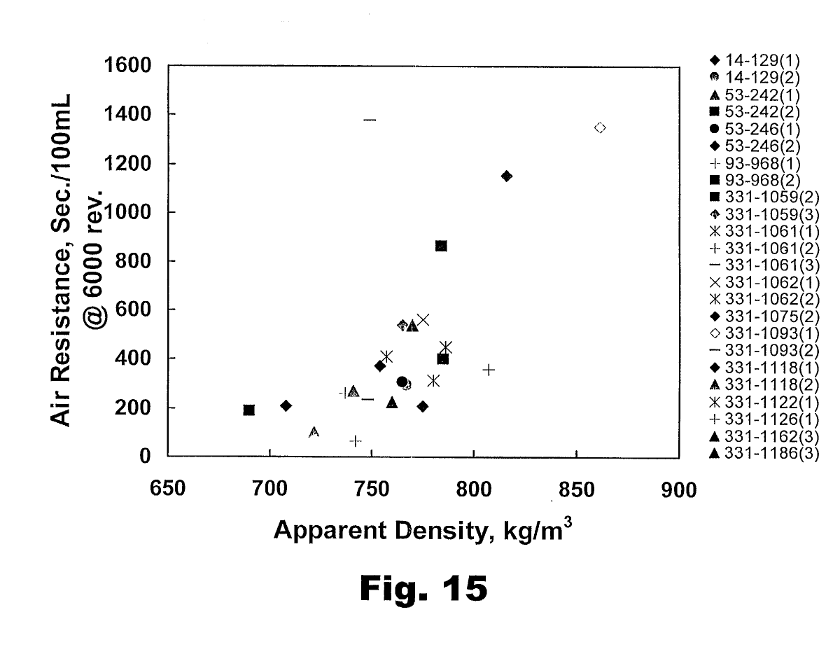

The wide range of cell sizes is further confirmed by the range of tensile indices observed at a given freeness, (a strongly negative relationship between tensile index and freeness properties exists Pearson coefficient −0.74, p=0.001; FIG. 14). Similarly the relationship of air resistance (Gurley) to sheet density, presented in FIG. 15, shows the wide ranging results consequent from cell wall property differences. For example, at beating levels of 6000 PFI revolutions, clones 331-1093 and 331-1075 exhibit the high tensile indices (116.1 and 113.1 N·m/g respectively) coupled with high air resistances (1364.7 and 1152.7 sec/100 mL respectively) which indicate that they possess thinner cell walls than do the other clonal pulps. By contrast, the pulp from clone 53-246 possesses the low tensile index and low air resistance values typical of a thicker cell-walled fiber (98.5 N·m/g, 256.9 sec/100 mL). Interestingly, the high calcium-containing pulp obtained from clone 331-1136 forms an outlier point for this analysis, exhibiting a combination of lower tensile strength (104.0 N·m/g) and very high air resistance (>30 min/100 mL). These variations mirror that seen in a separate study on a population of natural aspen clones. Again the potential for producing pulps for different end-use applications is clear and should be emphasized.

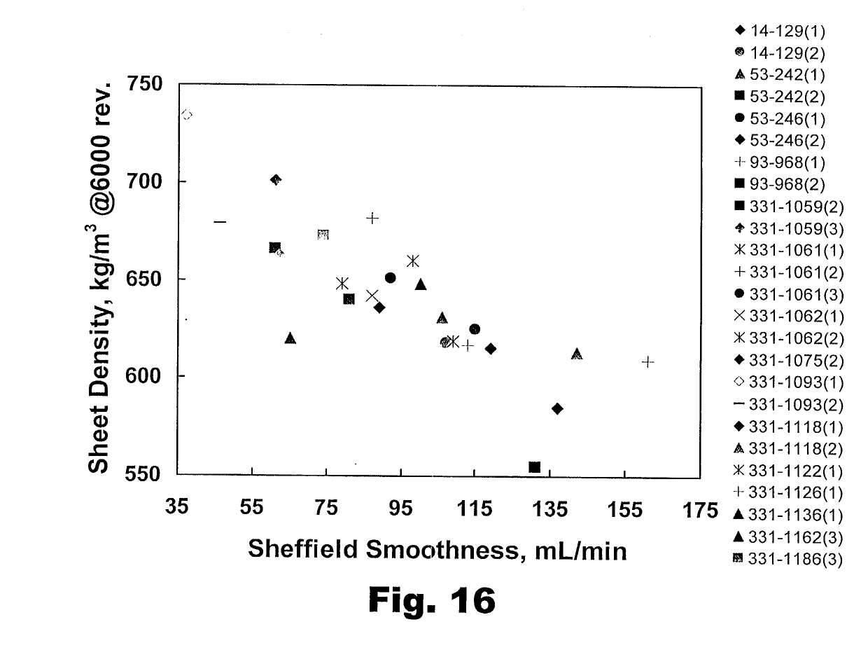

A number of the kraft pulping properties described here were used in a QTL mapping experiment to attempt to determine the chromosomal locations of any genes involved in the control of these important properties. The outcomes of this analysis are presented in the QTL mapping results section. In terms of sheet formation properties, smoothness shows significant relationships with freeness (Pearson coefficient 0.76, p=0.000) tensile strength (Pearson coefficient −0.87, p=0.000), and sheet density (Pearson coefficient −0.81, p=0.000; FIG. 16).

Optical Properties

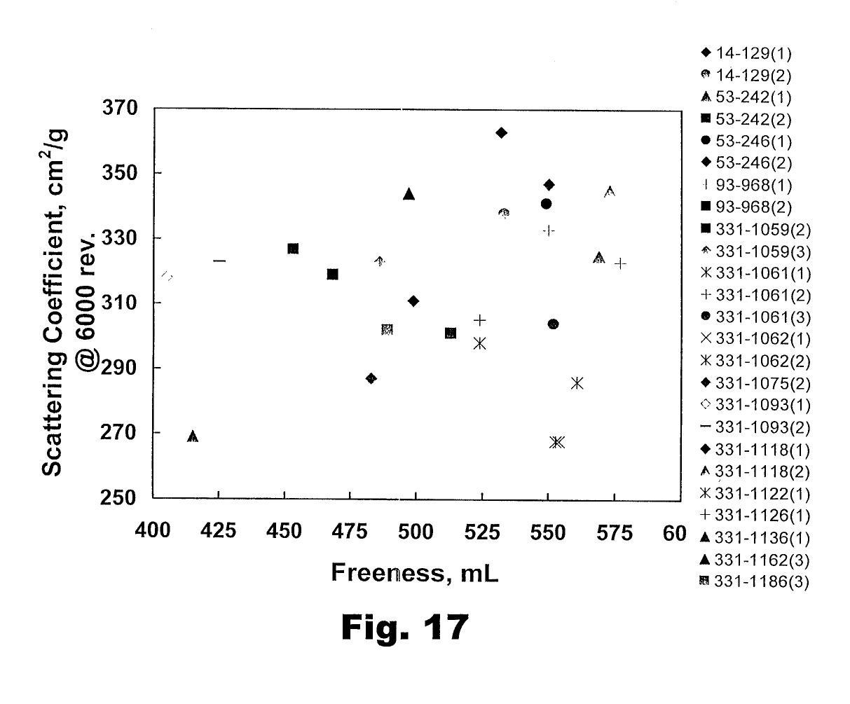

Hardwood kraft pulps principally impart optical and surface properties to paper rather than simply strength parameters. FIG. 17 shows the wide range of pulp scattering coefficients obtained from the unbleached clonal pulps at various freeness levels (at 0 PFI rev., the range is 268-363 cm²/g). A number of the pulps are exceptional (e.g. 331-1118)—even compared to aspen clones. For the purposes of comparison with the major competitive species, it should be noted that typical eucalypt pulps (Eucalyptus nitens samples) give scattering coefficients over a very similar range, 286-360 cm²/g.

3. Handsheet Analyses—Calcium Accumulation

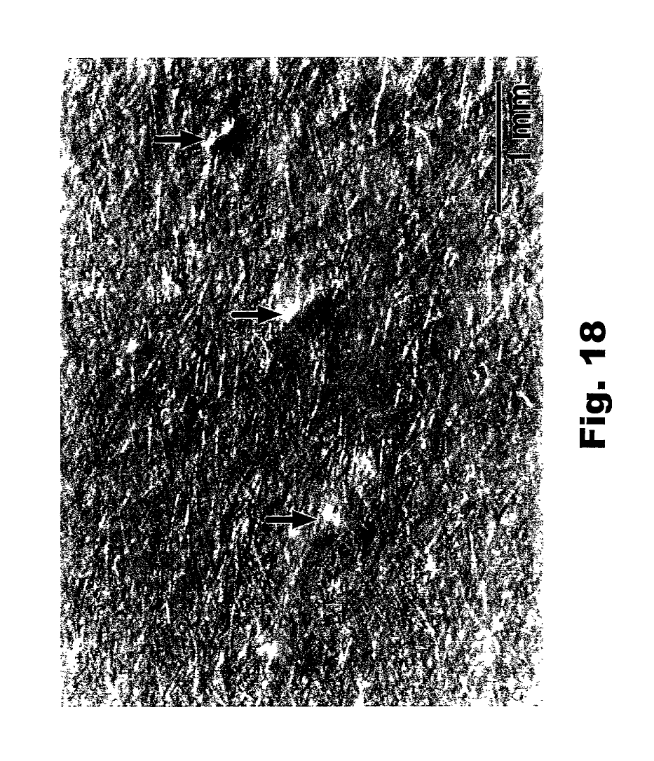

It was readily evident from a visual inspection of the resultant sheets that some unusual surface deformations, in the form of raised “bumps” approximately 1 mm in diameter, were prevalent (FIG. 18). The deformations were present in handsheets made after various levels of beating using standard PFI protocols (0-6000 rev.). It could also be observed that these deformations were present to a greater or lesser degree in the sheets dependent on the clonal source of the corresponding pulps. Sheets from the pulps were rated for the numbers of deformations using an arbitrary scale for visual inspection (similar to the ranking system used for assessing pest damage to hybrid poplars in pest-resistance QTL mapping studies. The ratings for each genotype analyzed are tabulated in Table XIV.

TABLE XIV
Arbitrary scale rating of degree of surface deformation
accumulation in test handsheets

| Genotype | Handsheet Deformation Rating | Number of Clones |
|---|---|---|
| ILL-29 | 1.5 | 2 |
| 93-968 | 3 | 2 |
| 53-246 | 2 | 2 |
| 53-242 | 3 | 2 |
| 331-1059 | 2.5 | 2 |
| 331-1061 | 2 | 3 |
| 331-1062 | 2.5 | 2 |
| 331-1075 | 0 | 1 |
| 331-1093 | 3 | 2 |
| 331-1118 | 3.5 | 2 |
| 331-1122 | 2 | 1 |
| 331-1126 | 0 | 1 |
| 331-1136 | 4 | 1 |
| 331-1162 | 3 | 1 |
| 331-1186 | 3 | 1 |

The results of the MAPMAKER-QTL 1.1 analysis performed using the phenotypic ranking data obtained from handsheet analyses (Table XIII) of each of the poplar clones are presented in Table XV below.

TABLE XV
Significant QTL detected for calcium deposition

| | | LOD | | Length/ | | |
|---|---|---|---|---|---|---|
| Trait | Marker/Linkage | Score | Phen % | cM | Weight | Dom. |
| Calcium | P1150-H07_10/N | 2.94 | 81.7 | 13.8 | 0.3286 | −1.7214 |
| deposits | | | | | | |

4. Microscopy and X-ray Analysis of Crystalline Deposits

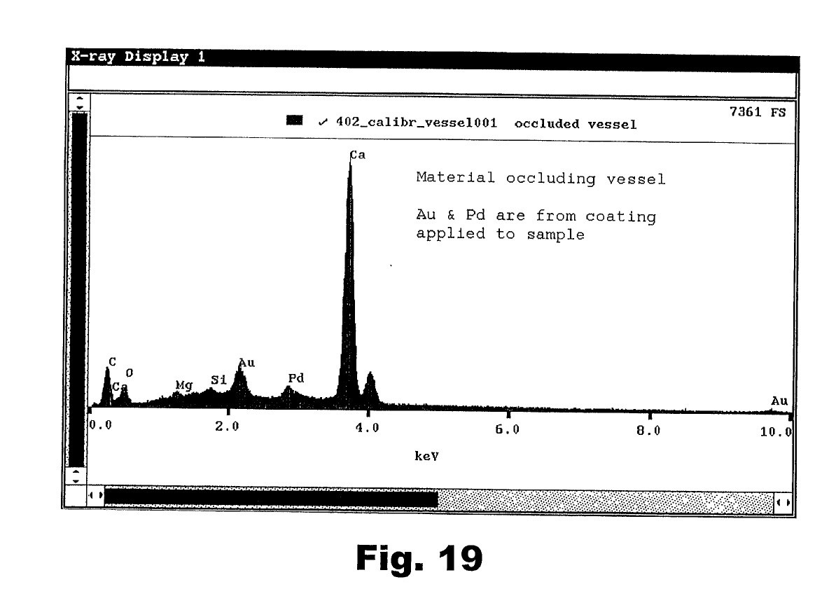

On further investigation, the deformations were found to be caused by a crystalline deposit found in some vessel elements in the pulp samples used to make the handsheets. These deposits were characterized by SEM/EDS and were found to consist primarily of calcium salts (FIG. 19).

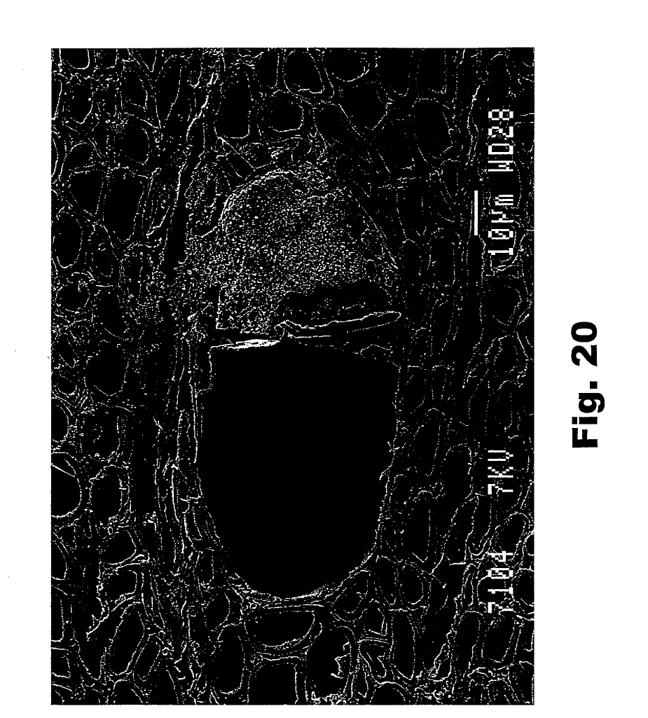

Examination of wood chips taken from the poplar clones by light microscopy and SEM also revealed the calcium deposits and, more intriguingly, their specific and exclusive nature. FIG. 20 shows an electron micrograph of two adjacent vessel elements in a wood chip, one of which is completely occluded with a deposit. By contrast, the adjacent element is completely free of crystals. Contrary to some literature reports, the deposits seen in this application (as examined microscopically) do not appear to be associated with any form of fungal attack or other decay process.

Alkaline Peroxide Refiner Mechanical Pulping

The raw data for the Alkaline Peroxide Refiner Mechanical Pulping (APRMP) from each of 15 hybrid poplar clones consisting of 24 hybrid poplar trees are shown in Table XVI.

TABLE XVI
Properties of APRMP Pulps from Hybrid Poplars

| | 14-129 (1) | | | 14-129 (2) | | |
|---|---|---|---|---|---|---|
| | 1466-4 | 1466-3 | 1466-2 | 1473-4 | 1473-3 | 1473-2 |
| Unscreened CSF (mL) | 202 | 263 | 378 | 178 | 195 | 259 |
| Specific Energy (MJ/kg) | 5.9 | 5.0 | 3.9 | 4.2 | 3.7 | 3.1 |
| Screened CSF (mL) | 208 | 274 | 408 | 181 | 206 | 266 |
| Reject (% o.d. pulp) | 0.0 | 0.0 | 0.1 | 0.0 | 0.0 | 0.1 |
| Apparent Sheet Density (kg/m³) | 388 | 380 | 350 | 464 | 458 | 439 |
| Burst Index (Kpa · m²/g) | 2.0 | 1.8 | 1.5 | 2.7 | 2.6 | 2.5 |
| Breaking length (km) | 4.0 | 3.8 | 2.9 | 5.1 | 4.8 | 4.4 |
| Tensile Index (N · m/g) | 39.1 | 36.8 | 28.4 | 50.1 | 47.5 | 42.8 |
| Stretch (%) | 1.57 | 1.49 | 1.16 | 1.97 | 1.83 | 1.66 |
| Tear Index (mN · m²/g) (4-Ply) | 5.5 | 5.7 | 5.1 | 6.1 | 6.3 | 6.3 |
| Sheffield Roughness (SU) | 137 | 167 | 268 | 105 | 115 | 123 |
| Brightness (%) | 78 | 79 | 79 | 77 | 78 | 78 |
| Opacity (%) | 85.5 | 85.0 | 84.5 | 82.4 | 81.4 | 81.6 |
| Scattering Coefficient (cm²/g) | 510 | 506 | 503 | 416 | 416 | 418 |
| R - 48 fraction (%) | 43.6 | 46.1 | 50.0 | 43.4 | 43.2 | 44.6 |
| Fines (P-200) (%) | 14.1 | 13.1 | 12.0 | 14.1 | 13.9 | 14.2 |
| W. Weighted Average Fibre Length (mm) | 1.00 | 1.06 | 1.20 | 0.99 | 0.97 | 1.03 |
| L. Weighted Average Fibre Length (mm) | 0.78 | 0.80 | 0.84 | 0.78 | 0.78 | 0.79 |
| Arithmetic Average Fibre Length (mm) | 0.54 | 0.54 | 0.54 | 0.54 | 0.54 | 0.54 |
| | 53-242 (1) | | | 53-242 (2) | | |
| | 1458-4 | 1458-3 | 1458-2 | 1452-4 | 1452-3 | 1452-2 |
| Unscreened CSF (mL) | 215 | 250 | 373 | 207 | 269 | 380 |
| Specific Energy (MJ/kg) | 6.8 | 6.1 | 4.9 | 6.8 | 5.7 | 4.4 |
| Screened CSF (mL) | 211 | 275 | 372 | 220 | 262 | 378 |
| Reject (% o.d. pulp) | 0.0 | 0.0 | 0.2 | 0.0 | 0.0 | 0.2 |
| Apparent Sheet Density (kg/m³) | 390 | 377 | 359 | 395 | 386 | 364 |
| Burst Index (Kpa · m²/g) | 2.1 | 2.0 | 1.8 | 2.1 | 2.0 | 1.7 |
| Breaking length (km) | 3.9 | 3.5 | 3.3 | 4.0 | 3.7 | 3.5 |
| Tensile Index (N · m/g) | 38.5 | 34.7 | 32.5 | 39.2 | 36.3 | 34.1 |
| Stretch (%) | 1.67 | 1.40 | 1.44 | 1.52 | 1.38 | 1.41 |
| Tear Index (mN · m²/g) (4-Ply) | 5.7 | 5.8 | 6.1 | 5.3 | 5.4 | 5.5 |
| Sheffield Roughness (SU) | 133 | 156 | 227 | 126 | 158 | 237 |
| Brightness (%) | 75 | 76 | 76 | 75 | 76 | 76 |
| Opacity (%) | 86.5 | 86.0 | 85.2 | 86.9 | 85.8 | 85.2 |
| Scattering Coefficient (cm²/g) | 498 | 498 | 489 | 500 | 492 | 482 |
| R - 48 fraction (%) | 49.1 | 49.2 | 54.1 | 45.4 | 47.2 | 52.5 |
| Fines (P-200) (%) | 16.9 | 17.2 | 14.1 | 14.8 | 14.4 | 12.5 |
| W. Weighted Average Fibre Length (mm) | 1.06 | 1.08 | 1.11 | 0.97 | 1.00 | 1.12 |
| L. Weighted Average Fibre Length (mm) | 0.84 | 0.84 | 0.86 | 0.77 | 0.78 | 0.81 |
| Arithmetic Average Fibre Length (mm) | 0.57 | 0.56 | 0.57 | 0.52 | 0.53 | 0.54 |
| | 53-246 (1) | | | 53-246 (2) | | |
| | 1472-4 | 1472-3 | 1472-2 | 1460-4 | 1461-3 | 1461-2 |
| Unscreened CSF (mL) | 198 | 237 | 372 | 221 | 308 | 388 |
| Specific Energy (MJ/kg) | 5.2 | 4.4 | 3.2 | 6.5 | 5.8 | 4.5 |
| Screened CSF (mL) | 184 | 236 | 374 | 227 | 326 | 416 |
| Reject (% o.d. pulp) | 0.1 | 0.1 | 0.7 | 0.0 | 0.1 | 0.5 |
| Apparent Sheet Density (kg/m³) | 425 | 403 | 382 | 440 | 401 | 374 |
| Burst Index (Kpa · m²/g) | 2.6 | 2.4 | 2.0 | 2.3 | 2.1 | 1.8 |
| Breaking length (km) | 4.6 | 4.3 | 3.8 | 4.4 | 3.8 | 3.3 |
| Tensile Index (N · m/g) | 44.7 | 42.1 | 37.1 | 42.8 | 37.6 | 32.1 |
| Stretch (%) | 1.89 | 1.64 | 1.51 | 1.99 | 1.69 | 1.37 |
| Tear Index (mN · m²/g) (4-Ply) | 6.8 | 6.5 | 6.5 | 6.2 | 6.3 | 6.4 |
| Sheffield Roughness (SU) | 117 | 122 | 213 | 110 | 152 | 231 |
| Brightness (%) | 79 | 79 | 79 | 76 | 76 | 77 |
| Opacity (%) | 82.5 | 81.7 | 81.5 | 86.8 | 85.8 | 85.1 |
| Scattering Coefficient (cm²/g) | 435 | 428 | 427 | 501 | 488 | 473 |
| R - 48 fraction (%) | 46.5 | 48.8 | 52.5 | 47.4 | 50.2 | 55.4 |
| Fines (P-200) (%) | 15.0 | 13.4 | 12.2 | 14.9 | 15.4 | 11.3 |
| W. Weighted Average Fibre Length (mm) | 1.05 | 1.11 | 1.16 | 1.02 | 1.15 | 1.19 |
| L. Weighted Average Fibre Length (mm) | 0.81 | 0.83 | 0.86 | 0.82 | 0.87 | 0.89 |
| Arithmetic Average Fibre Length (mm) | 0.54 | 0.55 | 0.55 | 0.55 | 0.56 | 0.56 |
| | 93-968 (1) | | | 93-968 (2) | | |
| | 1459-5 | 1459-4 | 1459-3 | 1450-3 | 1450-2 | 1451-2 |
| Unscreened CSF (mL) | 246 | 315 | 382 | 222 | 325 | 382 |
| Specific Energy (MJ/kg) | 8.5 | 7.3 | 6.1 | 5.6 | 4.5 | 3.8 |
| Screened CSF (mL) | 256 | 304 | 377 | 236 | 344 | 398 |
| Reject (% o.d. pulp) | 0.0 | 0.1 | 0.1 | 0.0 | 0.1 | 0.9 |
| Apparent Sheet Density (kg/m³) | 399 | 368 | 361 | 405 | 379 | 357 |
| Burst Index (Kpa · m²/g) | 2.2 | 1.9 | 1.8 | 2.2 | 1.9 | 1.8 |
| Breaking length (km) | 4.1 | 3.7 | 3.4 | 4.2 | 3.5 | 3.5 |
| Tensile Index (N · m/g) | 39.8 | 36.3 | 33.1 | 41.2 | 34.6 | 34.3 |
| Stretch (%) | 1.82 | 1.54 | 1.40 | 1.51 | 1.33 | 1.28 |
| Tear Index (mN · m²/g) (4-Ply) | 6.1 | 5.9 | 6.2 | 5.9 | 5.7 | 5.7 |
| Sheffield Roughness (SU) | 127 | 169 | 216 | 124 | 194 | 245 |
| Brightness (%) | 75 | 75 | 76 | 74 | 75 | 75 |
| Opacity (%) | 89.1 | 88.6 | 87.1 | 88.1 | 87.1 | 85.9 |
| Scattering Coefficient (cm²/g) | 534 | 528 | 510 | 522 | 516 | 487 |
| R - 48 fraction (%) | 43.6 | 51.3 | 56.5 | 45.4 | 50.9 | 54.5 |
| Fines (P-200) (%) | 15.5 | 13.5 | 12.6 | 15.5 | 14.0 | 12.5 |
| W. Weighted Average Fibre Length (mm) | 1.09 | 1.15 | 1.22 | 1.05 | 1.09 | 1.28 |
| L. Weighted Average Fibre Length (mm) | 0.87 | 0.89 | 0.92 | 0.81 | 0.83 | 0.90 |
| Arithmetic Average Fibre Length (mm) | 0.61 | 0.60 | 0.61 | 0.56 | 0.56 | 0.58 |
| | 331-1059 (2) | | | 331-1059 (3) | | |
| | 1453-3 | 1457-3 | 1453-2 | 1454-3 | 1455-3 | 1455-2 |
| Unscreened CSF (mL) | 210 | 249 | 329 | 216 | 239 | 312 |
| Specific Energy (MJ/kg) | 8.9 | 7.8 | 7.2 | 9.1 | 8.5 | 7.4 |
| Screened CSF (mL) | 230 | 257 | 336 | 212 | 250 | 314 |
| Reject (% o.d. pulp) | 0.1 | 0.6 | 0.8 | 0.3 | 0.8 | 1.9 |
| Apparent Sheet Density (kg/m³) | 378 | 363 | 352 | 376 | 350 | 350 |
| Burst Index (Kpa · m²/g) | 2.2 | 2.2 | 1.9 | 2.3 | 2.2 | 2.0 |
| Breaking length (km) | 3.9 | 3.8 | 3.5 | 4.2 | 4.0 | 3.7 |
| Tensile Index (N · m/g) | 38.5 | 37.6 | 33.9 | 40.9 | 38.7 | 36.3 |
| Stretch (%) | 1.84 | 1.70 | 1.58 | 2.01 | 1.89 | 1.65 |
| Tear Index (mN · m²/g) (4-Ply) | 5.1 | 6.3 | 5.7 | 6.2 | 6.3 | 6.2 |
| Sheffield Roughness (SU) | 138 | 151 | 181 | 143 | 157 | 187 |
| Brightness (%) | 75 | 75 | 76 | 78 | 78 | 78 |
| Opacity (%) | 88.7 | 87.4 | 87.1 | 87.4 | 86.5 | 86.5 |
| Scattering Coefficient (cm²/g) | 559 | 518 | 528 | 548 | 537 | 530 |
| R - 48 fraction (%) | 46.8 | 51.2 | 51.0 | 49.2 | 50.4 | 53.6 |
| Fines (P-200) (%) | 17.1 | 15.9 | 16.2 | 16.6 | 17.6 | 14.0 |
| W. Weighted Average Fibre Length (mm) | 1.03 | 1.18 | 1.14 | 1.07 | 1.16 | 1.20 |
| L. Weighted Average Fibre Length (mm) | 0.78 | 0.82 | 0.81 | 0.79 | 0.81 | 0.83 |
| Arithmetic Average Fibre Length (mm) | 0.51 | 0.51 | 0.52 | 0.52 | 0.52 | 0.52 |
| | 331-1061 (1) | | | 331-1061 (2) | | |
| | 1476-4 | 1476-3 | 1476-2 | 1474-4 | 1474-3 | 1474-2 |
| Unscreened CSF (mL) | 169 | 237 | 357 | 194 | 265 | 383 |
| Specific Energy (MJ/kg) | 5.0 | 4.0 | 3.0 | 6.0 | 5.1 | 3.9 |
| Screened CSF (mL) | 190 | 248 | 380 | 205 | 264 | 375 |
| Reject (% o.d. pulp) | 0.0 | 0.1 | 0.3 | 0.0 | 0.1 | 0.3 |
| Apparent Sheet Density (kg/m³) | 426 | 399 | 390 | 386 | 381 | 356 |
| Burst Index (Kpa · m²/g) | 2.7 | 2.4 | 2.1 | 2.2 | 2.2 | 1.9 |
| Breaking length (km) | 4.9 | 4.2 | 3.7 | 4.5 | 3.9 | 3.5 |
| Tensile Index (N · m/g) | 48.2 | 41.0 | 36.6 | 44.2 | 38.4 | 34.2 |
| Stretch (%) | 1.81 | 1.39 | 1.40 | 1.83 | 1.40 | 1.32 |
| Tear Index (mN · m²/g) (4-Ply) | 6.2 | 5.6 | 6.1 | 5.6 | 5.7 | 5.7 |
| Sheffield Roughness (SU) | 99 | 130 | 219 | 130 | 156 | 239 |
| Brightness (%) | 76 | 77 | 78 | 76 | 77 | 78 |
| Opacity (%) | 80.5 | 80.5 | 79.8 | 85.7 | 84.2 | 83.8 |
| Scattering Coefficient (cm²/g) | 387 | 394 | 391 | 482 | 471 | 465 |
| R - 48 fraction (%) | 48.1 | 50.2 | 53.8 | 46.9 | 49.3 | 56.0 |
| Fines (p-200) (%) | 15.1 | 14.9 | 9.8 | 14.5 | 11.9 | 12.7 |
| W. Weighted Average Fibre Length (mm) | 1.07 | 1.11 | 1.19 | 1.06 | 1.08 | 1.21 |
| L. Weighted Average Fibre Length (mm) | 0.83 | 0.86 | 0.89 | 0.78 | 0.79 | 0.84 |
| Arithmetic Average Fibre Length (mm) | 0.54 | 0.56 | 0.57 | 0.52 | 0.53 | 0.53 |
| | 331-1061 (3) | | | 331-1062 (1) | | |
| | 1475-5 | 1475-4 | 1475-3 | 1456-4 | 1456-3 | 1456-2 |
| Unscreened CSF (mL) | 219 | 273 | 363 | 220 | 247 | 361 |
| Specific Energy (MJ/kg) | 7.3 | 6.3 | 5.1 | 7.0 | 6.2 | 4.9 |
| Screened CSF (mL) | 226 | 301 | 371 | 231 | 270 | 359 |
| Reject (% o.d. pulp) | 0.0 | 0.1 | 0.1 | 0.0 | 0.1 | 0.5 |
| Apparent Sheet Density (kg/m³) | 359 | 354 | 336 | 374 | 370 | 349 |
| Burst Index (Kpa · m²/g) | 1.9 | 1.7 | 1.6 | 1.9 | 1.9 | 1.6 |
| Breaking length (km) | 3.4 | 3.3 | 2.9 | 3.6 | 3.4 | 3.2 |
| Tensile Index (N · m/g) | 33.5 | 31.9 | 28.2 | 35.5 | 33.2 | 31.4 |
| Stretch (%) | 1.25 | 1.35 | 1.15 | 1.43 | 1.31 | 1.34 |
| Tear Index (mN · m²/g) (4-Ply) | 5.0 | 5.0 | 4.9 | 5.5 | 5.6 | 5.7 |
| Sheffield Roughness (SU) | 168 | 219 | 276 | 132 | 156 | 225 |
| Brightness (%) | 78 | 79 | 80 | 77 | 77 | 77 |
| Opacity (%) | 84.9 | 83.7 | 83.0 | 86.2 | 85.8 | 84.7 |
| Scattering Coefficient (cm²/g) | 490 | 478 | 466 | 498 | 501 | 482 |
| R - 48 fraction (%) | 48.0 | 54.0 | 56.1 | 51.6 | 53.7 | 57.2 |
| Fines (P-200) (%) | 15.4 | 13.6 | 11.4 | 17.4 | 17.0 | 13.5 |
| W. Weighted Average Fibre Length (mm) | 1.04 | 1.06 | 1.18 | 1.13 | 1.22 | 1.30 |
| L. Weighted Average Fibre Length (mm) | 0.82 | 0.81 | 0.85 | 0.87 | 0.89 | 0.92 |
| Arithmetic Average Fibre Length (mm) | 0.52 | 0.53 | 0.53 | 0.55 | 0.55 | 0.56 |
| | 331-1062 (2) | | | 331-1075 (2) | | |
| | 1462-4 | 1462-3 | 1462-2 | 1444-4 | 1444-3 | 1446 |
| Unscreened CSF (mL) | 209 | 273 | 351 | 237 | 284 | 411 |
| Specific Energy (MJ/kg) | 5.2 | 4.3 | 3.5 | 10.8 | 9.5 | 7.9 |
| Screened CSF (mL) | 225 | 289 | 359 | 250 | 297 | 422 |
| Reject (% o.d. pulp) | 0.0 | 0.0 | 0.1 | 0.1 | 0.1 | 0.3 |
| Apparent Sheet Density (kg/m³) | 409 | 397 | 386 | 344 | 324 | 309 |
| Burst Index (Kpa · m²/g) | 2.1 | 2.1 | 1.9 | 1.7 | 1.6 | 1.3 |
| Breaking length (km) | 4.1 | 4.1 | 3.7 | 3.3 | 2.8 | 2.5 |
| Tensile Index (N · m/g) | 40.6 | 40.3 | 36.3 | 32.0 | 27.4 | 24.8 |
| Stretch (%) | 1.39 | 1.46 | 1.29 | 1.36 | 1.21 | 1.23 |
| Tear Index (mN · m²/g) (4-Ply) | 5.4 | 5.4 | 5.4 | 4.8 | 4.7 | 4.3 |
| Sheffield Roughness (SU) | 116 | 135 | 208 | 182 | 235 | 306 |
| Brightness (%) | 77 | 77 | 78 | 75 | 75 | 76 |
| Opacity (%) | 85.4 | 84.0 | 83.4 | 89.3 | 88.6 | 88.1 |
| Scattering Coefficient (cm²/g) | 492 | 460 | 458 | 577 | 556 | 549 |
| R - 48 fraction (%) | 47.2 | 48.6 | 52.9 | 41.0 | 46.4 | 50.0 |
| Fines (P-200) (%) | 15.7 | 15.8 | 13.1 | 18.6 | 17.3 | 14.2 |
| W. Weighted Average Fibre Length (mm) | 1.07 | 1.15 | 1.10 | 0.99 | 1.07 | 1.15 |
| L. Weighted Average Fibre Length (mm) | 0.83 | 0.87 | 0.85 | 0.78 | 0.80 | 0.83 |
| Arithmetic Average Fibre Length (mm) | 0.56 | 0.58 | 0.56 | 0.54 | 0.54 | 0.54 |
| | 331-1093 (1) | | | 331-1093 (2) | | |
| | 1470-4 | 1470-3 | 1470-2 | 1467-4 | 1467-3 | 1467-2 |
| Unscreened CSF (mL) | 160 | 200 | 295 | 184 | 210 | 275 |
| Specific Energy (MJ/kg) | 5.7 | 5.0 | 4.0 | 4.6 | 4.1 | 3.4 |
| Screened CSF (mL) | 171 | 214 | 305 | 192 | 220 | 292 |
| Reject (% o.d. pulp) | 0.0 | 0.1 | 0.4 | 0.0 | 0.0 | 0.1 |
| Apparent Sheet Density (kg/m³) | 384 | 381 | 353 | 427 | 424 | 413 |
| Burst Index (Kpa · m²/g) | 2.4 | 2.2 | 2.0 | 2.5 | 2.4 | 2.1 |
| Breaking length (km) | 4.5 | 4.3 | 3.8 | 4.7 | 4.6 | 4.1 |
| Tensile Index (N · m/g) | 44.5 | 41.7 | 36.8 | 46.3 | 44.8 | 40.5 |
| Stretch (%) | 1.67 | 1.55 | 1.50 | 1.64 | 1.63 | 1.53 |
| Tear Index (mN · m²/g) (4-Ply) | 5.8 | 6.1 | 5.7 | 5.5 | 5.6 | 5.5 |
| Sheffield Roughness (SU) | 120 | 137 | 186 | 108 | 127 | 159 |
| Brightness (%) | 74 | 75 | 76 | 79 | 78 | 79 |
| Opacity (%) | 86.5 | 86.3 | 85.8 | 84.4 | 83.8 | 84.0 |
| Scattering Coefficient (cm²/g) | 522 | 506 | 506 | 493 | 484 | 495 |
| R - 48 fraction (%) | 44.2 | 46.4 | 49.6 | 39.0 | 42.0 | 45.3 |
| Fines (P-200) (%) | 16.6 | 14.8 | 13.2 | 15.6 | 13.4 | 11.4 |
| W. Weighted Average Fibre Length (mm) | 1.04 | 1.06 | 1.21 | 0.96 | 0.97 | 1.00 |
| L. Weighted Average Fibre Length (mm) | 0.74 | 0.75 | 0.79 | 0.73 | 0.73 | 0.74 |
| Arithmetic Average Fibre Length (mm) | 0.51 | 0.51 | 0.52 | 0.52 | 0.52 | 0.52 |
| | 331-1118 (1) | | | 331-1118 (2) | | |
| | 1468-3 | 1468-2 | 1469-2 | 1471-4 | 1471-3 | 1471-2 |
| Unscreened CSF (mL) | 149 | 191 | 283 | 184 | 223 | 358 |
| Specific Energy (MJ/kg) | 4.2 | 3.8 | 3.0 | 6.5 | 5.5 | 4.3 |
| Screened CSF (mL) | 159 | 200 | 296 | 197 | 240 | 383 |
| Reject (% o.d. pulp) | 0.0 | 0.0 | 0.4 | 0.0 | 0.0 | 0.3 |
| Apparent Sheet Density (kg/m³) | 463 | 458 | 393 | 376 | 358 | 340 |
| Burst Index (Kpa · m²/g) | 2.9 | 2.8 | 2.2 | 2.2 | 2.0 | 1.7 |
| Breaking length (km) | 5.3 | 5.0 | 4.3 | 4.1 | 3.6 | 3.1 |
| Tensile Index (N · m/g) | 51.7 | 49.5 | 41.7 | 40.2 | 35.1 | 30.7 |
| Stretch (%) | 1.90 | 1.83 | 1.65 | 1.69 | 1.41 | 1.25 |
| Tear Index (mN · m²/g) (4-Ply) | 6.0 | 6.0 | 6.3 | 5.6 | 5.6 | 6.1 |
| Sheffield Roughness (SU) | 103 | 113 | 161 | 135 | 164 | 264 |
| Brightness (%) | 77 | 78 | 78 | 77 | 77 | 78 |
| Opacity (%) | 83.3 | 82.0 | 82.3 | 86.2 | 86.2 | 85.3 |
| Scattering Coefficient (cm²/g) | 431 | 429 | 439 | 508 | 520 | 496 |
| R - 48 fraction (%) | 40.5 | 40.8 | 47.6 | 42.3 | 45.7 | 48.5 |
| Fines (P-200) (%) | 13.7 | 13.6 | 12.1 | 17.2 | 15.0 | 14.3 |
| W. Weighted Average Fibre Length (mm) | 0.97 | 0.96 | 1.11 | 1.01 | 1.07 | 1.15 |
| L. Weighted Average Fibre Length (mm) | 0.74 | 0.74 | 0.78 | 0.77 | 0.78 | 0.80 |
| Arithmetic Average Fibre Length (mm) | 0.52 | 0.52 | 0.53 | 0.53 | 0.53 | 0.54 |
| | 331-1122 (1) | | | 331-1126 (1) | | |
| | 1447-4 | 1447-3 | 1448-2 | 1465-4 | 1465-3 | 1465-2 |
| Unscreened CSF (mL) | 210 | 300 | 425 | 191 | 255 | 379 |
| Specific Energy (MJ/kg) | 7.3 | 6.1 | 4.3 | 6.3 | 5.2 | 4.0 |
| Screened CSF (mL) | 227 | 313 | 420 | 202 | 267 | 403 |
| Reject (% o.d. pulp) | 0.1 | 0.2 | 0.7 | 0.0 | 0.0 | 0.2 |
| Apparent Sheet Density (kg/m³) | 360 | 339 | 327 | 363 | 345 | 320 |
| Burst Index (Kpa · m²/g) | 1.7 | 1.6 | 1.4 | 1.8 | 1.7 | 1.4 |
| Breaking length (km) | 3.6 | 3.3 | 2.8 | 3.5 | 3.3 | 2.8 |
| Tensile Index (N · m/g) | 35.8 | 31.9 | 27.2 | 34.8 | 31.9 | 27.1 |
| Stretch (%) | 1.33 | 1.37 | 1.11 | 1.45 | 1.40 | 1.25 |
| Tear Index (mN · m²/g) (4-Ply) | 4.0 | 3.9 | 3.8 | 4.8 | 5.0 | 5.0 |
| Sheffield Roughness (SU) | 144 | 220 | 290 | 164 | 221 | 304 |
| Brightness (%) | 75 | 75 | 76 | 75 | 75 | 76 |
| Opacity (%) | 88.0 | 87.2 | 86.3 | 86.3 | 86.2 | 85.2 |
| Scattering Coefficient (cm²/g) | 533 | 518 | 506 | 505 | 497 | 480 |
| R - 48 fraction (%) | 45.6 | 54.1 | 56.0 | 44.3 | 48.6 | 52.1 |
| Fines (P-200) (%) | 15.8 | 14.0 | 11.3 | 20.3 | 15.8 | 14.9 |
| W. Weighted Average Fibre Length (mm) | 1.00 | 1.06 | 1.25 | 1.08 | 1.10 | 1.24 |
| L. Weighted Average Fibre Length (mm) | 0.75 | 0.78 | 0.84 | 0.85 | 0.87 | 0.90 |
| Arithmetic Average Fibre Length (mm) | 0.49 | 0.52 | 0.52 | 0.58 | 0.58 | 0.59 |
| | 331-1162 (3) | | | 331-1186 (3) | | |
| | 1464-4 | 1464-3 | 1464-2 | 1449-4 | 1449-3 | 1449-2 |
| Unscreened CSF (mL) | 170 | 215 | 266 | 188 | 253 | 380 |
| Specific Energy (MJ/kg) | 5.4 | 4.8 | 4.0 | 7.6 | 6.5 | 5.1 |
| Screened CSF (mL) | 197 | 232 | 291 | 212 | 269 | 382 |
| Reject (% o.d. pulp) | 0.0 | 0.0 | 0.1 | 0.0 | 0.0 | 0.1 |
| Apparent Sheet Density (kg/m³) | 417 | 400 | 394 | 409 | 402 | 354 |
| Burst Index (Kpa · m²/g) | 1.9 | 1.8 | 1.8 | 2.4 | 2.1 | 1.7 |
| Breaking length (km) | 3.7 | 3.6 | 3.3 | 4.3 | 3.8 | 3.4 |
| Tensile Index (N · m/g) | 36.5 | 35.5 | 32.8 | 42.0 | 37.4 | 32.9 |
| Stretch (%) | 1.36 | 1.38 | 1.17 | 1.74 | 1.44 | 1.36 |
| Tear Index (mN · m²/g) (4-Ply) | 5.0 | 5.1 | 4.5 | 5.8 | 5.6 | 5.6 |
| Sheffield Roughness (SU) | 115 | 136 | 163 | 104 | 142 | 226 |
| Brightness (%) | 74 | 74 | 74 | 76 | 77 | 78 |
| Opacity (%) | 88.5 | 87.9 | 87.1 | 86.2 | 85.6 | 84.8 |
| Scattering Coefficient (cm²/g) | 530 | 514 | 518 | 505 | 500 | 499 |
| R - 48 fraction (%) | 45.3 | 45.4 | 46.5 | 46.4 | 48.7 | 51.6 |
| Fines (P-200) (%) | 19.5 | 14.1 | 14.1 | 16.0 | 16.1 | 14.6 |
| W. Weighted Average Fibre Length (mm) | 1.06 | 1.10 | 1.06 | 1.00 | 1.04 | 1.12 |
| L. Weighted Average Fibre Length (mm) | 0.84 | 0.86 | 0.84 | 0.79 | 0.80 | 0.83 |
| Arithmetic Average Fibre Length (mm) | 0.57 | 0.57 | 0.57 | 0.52 | 0.52 | 0.53 |

In general, appropriate baseline values of pulp freeness and specific refining energy are the two parameters commonly used to monitor mechanical and optical properties of APRMP pulps. Thus, to facilitate data analysis and discussion, the raw data were standardized by interpolation or extrapolation to a freeness of 200 mL CSF (Table XVII) and a specific refining energy (SRE) of 6.0 MJ/kg (Table XVIII).

TABLE XVII
Properties of APRMP Pulps from Hybrid Poplars at a Constant Freeness of 200 mL CSF

| | | | | Length | | | | | | | | |
|---|---|---|---|---|---|---|---|---|---|---|---|---|
| | Specific | | | Weighted | | | | | | | | |
| | Refining | R - 48 | Fines | Fiber | Sheet | Tensile | | | Bright- | Sheffield | Scattering | |
| | Energy | Fraction | (P-200) | Length | Density | Index | Stretch | Tear Index | ness | Roughness | Coefficient | Opacity |
| Hybrid No. | (MJ/kg) | (%) | (%) | (mm) | (kg/m³) | (N · m/g) | (%) | (mN · m²/g) | (%) | (SU) | (cm²/g) | (%) |
| 14-129 (1) | 5.9 | 43.5 | 14.0 | 0.78 | 392 | 40.0 | 1.60 | 5.5 | 78 | 130 | 510 | 85.5 |
| 14-129 (2) | 3.7 | 43.2 | 13.9 | 0.78 | 459 | 48.2 | 1.87 | 6.3 | 78 | 111 | 416 | 81.9 |
| 53-242 (1) | 7.0 | 48.0 | 17.4 | 0.81 | 392 | 39.0 | 1.68 | 5.7 | 75 | 128 | 498 | 86.6 |
| 53-242 (2) | 6.9 | 44.5 | 15.2 | 0.77 | 399 | 40.0 | 1.54 | 5.3 | 75 | 113 | 503 | 87.3 |
| 53-246 (1) | 5.2 | 47.3 | 14.5 | 0.81 | 417 | 43.8 | 1.80 | 6.7 | 79 | 118 | 432 | 82.2 |
| 53-246 (2) | 6.7 | 46.5 | 15.8 | 0.82 | 448 | 44.5 | 2.09 | 6.2 | 76 | 95 | 506 | 87.0 |
| 93-968 (1) | 9.3 | 40.0 | 17.4 | 0.85 | 413 | 42.5 | 1.94 | 6.1 | 75 | 90 | 547 | 90.1 |
| 93-968 (2) | 5.9 | 43.3 | 16.3 | 0.79 | 417 | 42.5 | 1.56 | 5.9 | 74 | 95 | 528 | 88.7 |
| 331-1059 (2) | 9.1 | 45.0 | 17.5 | 0.79 | 383 | 40.0 | 1.90 | 5.0 | 75 | 127 | 565 | 88.8 |
| 331-1059 (3) | 9.3 | 48.5 | 17.0 | 0.79 | 383 | 41.2 | 2.06 | 6.2 | 78 | 137 | 550 | 87.4 |
| 331-1061 (1) | 4.6 | 48.6 | 15.1 | 0.84 | 417 | 46.0 | 1.75 | 6.2 | 76 | 103 | 389 | 80.5 |
| 331-1061 (2) | 5.9 | 46.8 | 14.5 | 0.78 | 389 | 44.5 | 1.83 | 5.6 | 76 | 127 | 482 | 85.7 |
| 331-1061 (3) | 7.5 | 47.4 | 16.2 | 0.80 | 361 | 34.8 | 1.30 | 5.0 | 78 | 150 | 494 | 85.4 |
| 331-1062 (1) | 7.2 | 50.4 | 18.3 | 0.87 | 382 | 37.0 | 1.48 | 5.5 | 77 | 107 | 504 | 86.6 |
| 331-1062 (2) | 5.3 | 46.5 | 16.7 | 0.84 | 413 | 42.0 | 1.50 | 5.4 | 77 | 105 | 490 | 85.6 |
| 331-1075 (2) | 11.1 | 38.0 | 19.8 | 0.77 | 361 | 34.0 | 1.40 | 5.0 | 75 | 155 | 580 | 89.5 |
| 331-1093 (1) | 5.0 | 45.6 | 15.7 | 0.75 | 382 | 42.7 | 1.59 | 6.0 | 75 | 132 | 511 | 86.4 |
| 331-1093 (2) | 4.3 | 40.0 | 14.8 | 0.73 | 426 | 45.9 | 1.64 | 5.5 | 79 | 114 | 490 | 84.2 |
| 331-1118 (1) | 3.7 | 40.8 | 13.6 | 0.75 | 448 | 49.5 | 1.83 | 6.0 | 78 | 113 | 429 | 82.0 |
| 331-1118 (2) | 6.0 | 42.5 | 17.2 | 0.77 | 376 | 40.2 | 1.69 | 5.6 | 77 | 137 | 508 | 86.2 |
| 331-1122 (1) | 7.5 | 43.8 | 16.5 | 0.74 | 368 | 37.0 | 1.45 | 4.0 | 75 | 128 | 536 | 88.2 |
| 331-1126 (1) | 6.1 | 44.3 | 20.3 | 0.85 | 363 | 34.8 | 1.45 | 4.8 | 75 | 164 | 505 | 86.3 |
| 331-1162 (3) | 5.0 | 45.3 | 19.5 | 0.85 | 415 | 36.5 | 1.36 | 5.0 | 74 | 115 | 530 | 88.5 |
| 331-1186 (3) | 7.3 | 46.3 | 16.1 | 0.79 | 415 | 43.0 | 1.78 | 5.8 | 76 | 94 | 505 | 86.3 |

Specific Refining Energy

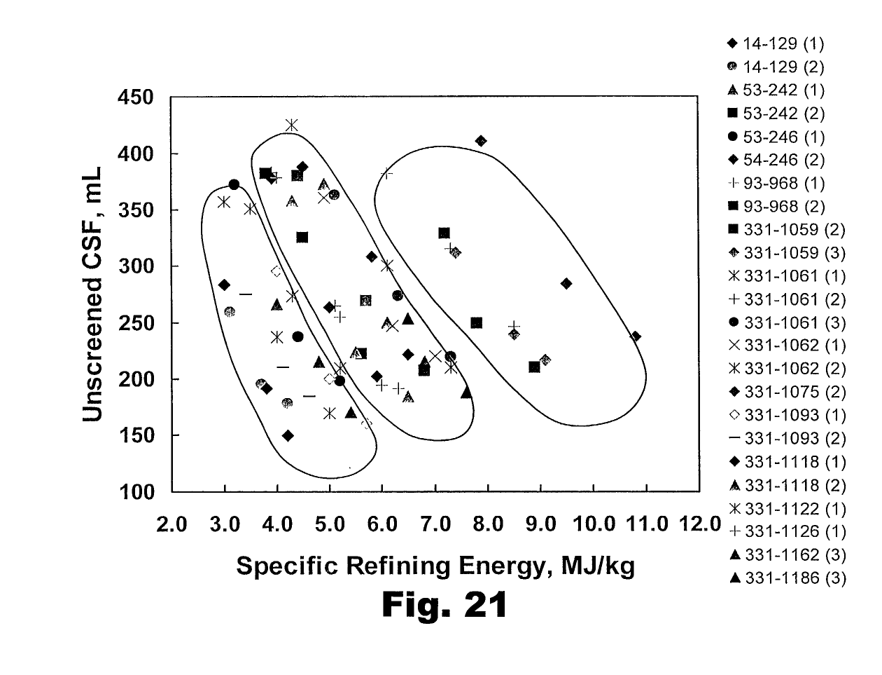

The specific refining energy consumed to reach a given freeness in the range of 150 to 425 mL CSF for the 24 hybrid poplar trees is shown in FIG. 21. The raw data show considerable scatter thanks largely to intraclonal variability which renders clonal effects non-significant (ANOVA p=0.067). Each set of points in FIG. 21 is surrounded by envelopes rather than a best-fit line or curve. The envelopes can be classified into three general groups as shown below.

| High SRE Group | Medium SRE Group | Low SRE Group |
|---|---|---|
| 93-968(1) | 14-129(1) | 14-129(2) |
| 331-1059(2) | 53-242(1) | 53-246(1) |
| 331-1059(3) | 53-242(2) | 331-1061(1) |
| 331-1075(2) | 53-246(2) | 331-1062(2) |
| | 93-968(2) | 331-1093(1) |
| | 331-1061(2) | 331-1093(2) |
| | 331-1061(3) | 331-1118(1) |
| | 331-1062(1) | 331-1162(3) |
| | 331-1118(2) | |
| | 331-1122(1) | |
| | 331-1126(1) | |
| | 331-1186(3) | |

The differences in SRE demand are more evident at 200 mL CSF as clones 93-968(1) and 331-1059(3) require 9.3 MJ/kg SRE whereas clones 14-129(2) and 331-1118(1) require 3.7 MJ/kg SRE or 60% of the energy demand (Table XVII). Clone 331-1075(2) is clearly exceptional as it required 11.1 MJ/kg of specific refining energy to the same freeness level. The three distinct SRE groups shown in FIG. 21 are consistent with previous observations of chemithermomechanical (CTMP) pulping of nine different “wild” aspen clones from Northeast British Columbia.

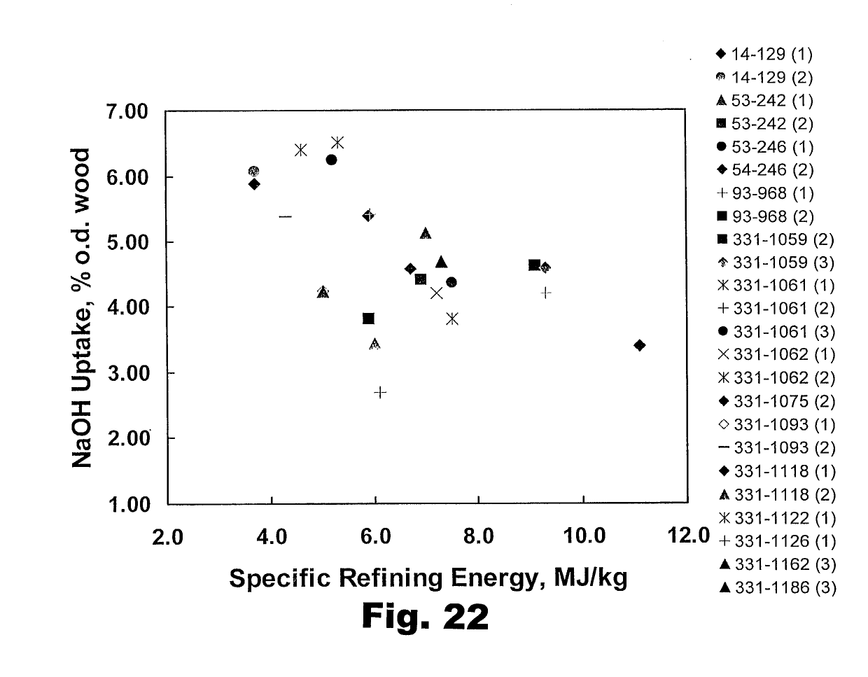

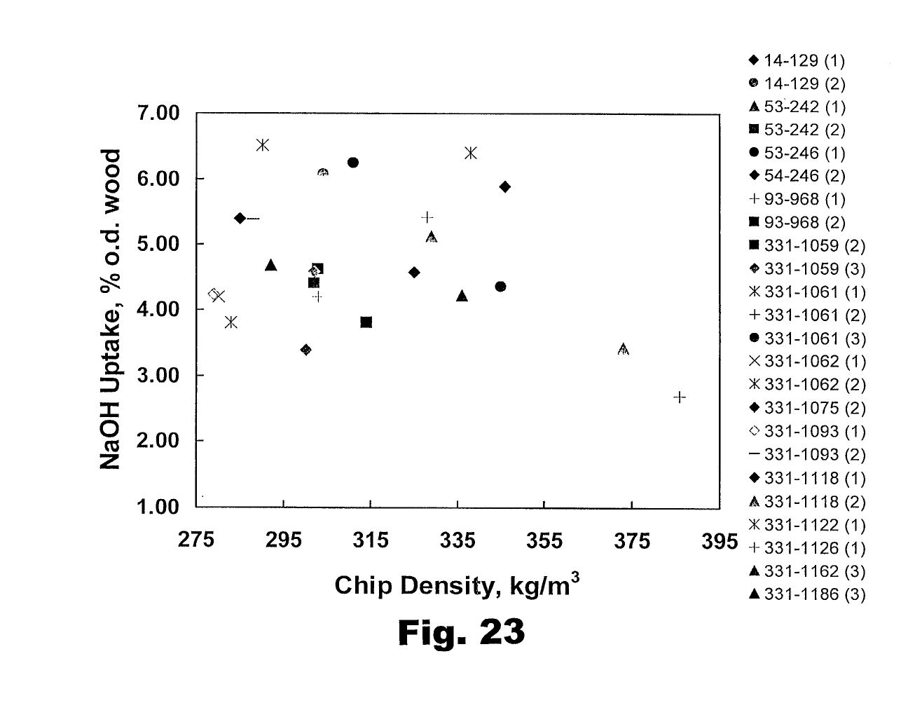

NaOH/H₂O₂ uptake for each tree are shown in Table XIX. The data indicate a much lower chemical uptake for the unusual high energy consumption clone 331-1075(2) than for the other clones investigated in this study. NaOH uptake values for each clone at 200 mL CSF are plotted against SRE in FIG. 22. FIG. 22 shows that high chemical uptake reduces energy demand at a given freeness of 200 mL. The significant negative relationship noted here (Pearson coefficient −0.526, p=0.025) agrees well with previous findings that SRE of hardwood mechanical pulps increases with diminishing chemical uptake, although the variability seen here is greater than that observed for aspen CTMP pulps. The reasons for intraclonal variability in chemical uptake are not clear. The most probable explanation for low chemical uptake by certain clones is likely a function of the cell wall thickness and lumen diameters of earlywood (large) and latewood (small). It has been reported that a thicker S1 wall makes it more difficult for the hardwood fiber to absorb chemical in order to swell and/or collapse. A plot of the NaOH uptake vs. chip density (FIG. 23) also confirms previous observations that wood density does not affect chemical uptake by Populus species chips and further contrasts with data suggesting that earlywood density affects chemical uptake for Eucalyptus nitens.

TABLE XIX
Chip density and chemical uptake for APRMP pulps
Chip thickness = 2-6 mm

| | Chip Density^{a} | NaOH | H₂O₂ |
|---|---|---|---|
| Sample No. | (kg/m³) | (% o.d. wood) | (% o.d. wood) |
| 14-129 (1) | 285 | 5.39 | 3.44 |
| 14-129 (2) | 304 | 6.07 | 3.88 |
| 53-242 (1) | 329 | 5.13 | 3.27 |
| 53-242 (2) | 302 | 4.41 | 2.82 |
| 53-246 (1) | 311 | 6.24 | 3.99 |
| 54-246 (2) | 325 | 4.57 | 2.92 |
| 93-968 (1) | 303 | 4.20 | 2.68 |
| 93-968 (2) | 314 | 3.80 | 2.43 |
| 331-1059 (2) | 303 | 4.63 | 2.95 |
| 331-1059 (3) | 302 | 4.59 | 2.93 |
| 331-1061 (1) | 338 | 6.40 | 4.09 |
| 331-1061 (2) | 328 | 5.41 | 3.46 |
| 331-1061 (3) | 345 | 4.35 | 2.78 |
| 331-1062 (1) | 280 | 4.20 | 2.68 |
| 331-1062 (2) | 290 | 6.51 | 4.24 |
| 331-1075 (2) | 300 | 3.39 | 2.16 |
| 331-1093 (1) | 279 | 4.23 | 2.70 |
| 331-1093 (2) | 288 | 5.38 | 3.43 |
| 331-1118 (1) | 346 | 5.89 | 3.76 |
| 331-1118 (2) | 373 | 3.42 | 2.18 |
| 331-1122 (1) | 283 | 3.80 | 2.43 |
| 331-1126 (1) | 386 | 2.69 | 1.72 |
| 331-1162 (3) | 336 | 4.22 | 2.69 |
| 331-1186 (3) | 292 | 4.69 | 3.00 |

Fiber Properties

As expected, the long-fiber fraction R-48 (retained on the 48-mesh screen of a Bauer-McNett fiber classifier) and LWFL (length-weighted fiber length) increased with increasing freeness and decreasing SRE, whereas the fines content P-200 (passed through the 200-mesh screen of a Bauer McNett fiber classifier) increased with decreasing freeness and increasing SRE as shown in Table XVII. The LWFL values obtained from the mechanical APRMP pulps at a freeness of 200 mL (Table XVII) show a significant correlation (Pearson coefficient 0.479, p=0.018) with the LWFL values observed for the chemical pulps (Table XI) obtained from the same clones. Unexpectedly, the LWFL values for APRMP pulps were consistently longer than those from the chemical pulps obtained from the same trees. The reasons for this observation is not clear. Perhaps, the alkali treatment of hybrid poplar have softened the middle lamella thus allowing the individual fibers to be peeled from the matrix in a longer and a more intact state in the refiner than those from the chemical pulping process.

Strength Properties and Sheet Consolidation

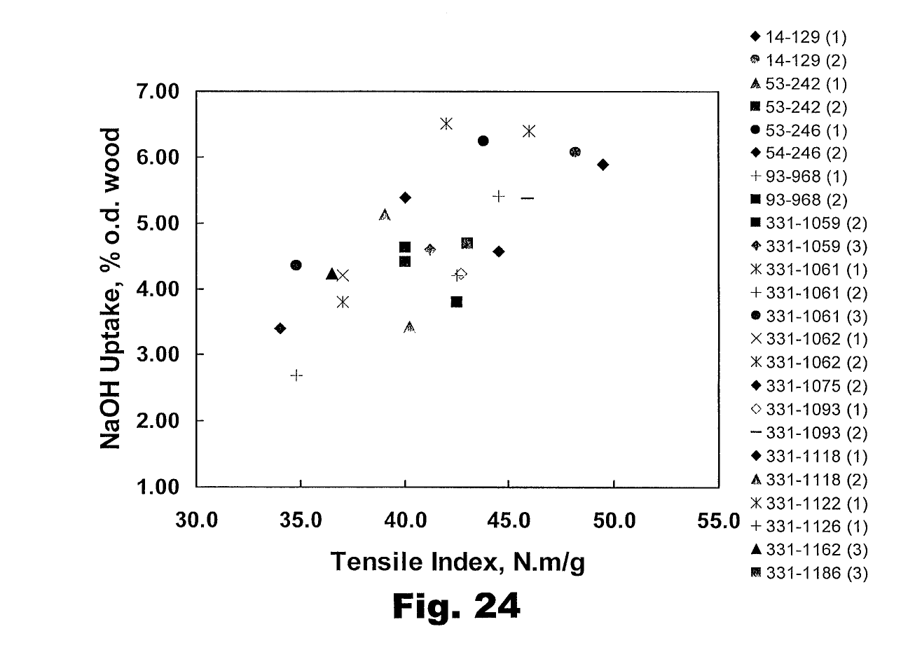

Tensile index increased with decreasing freeness, increasing sheet density, and increasing specific refining energy (Table XVI). In addition, LWFL also has a highly significant negative relationship with APRMP pulp tensile index (Pearson coefficient −0.74, p=0.001). In general, there is considerable variability in tensile strength from the various clones at a given freeness of 200 mL CSF and a given specific refining energy of 6.0 MJ/kg (Tables XVII and XVIII, respectively). At a given freeness of 200 mL CSF the tensile index values range from 34.0 to 49.5 N·m/g. There is also considerable interclonal variability in tensile strength, for example, the three individuals comprising the genotype clone 331-1061 have a mean tensile index of 41.8 N·m/g with a standard deviation of 5.0 N·m/g at a given freeness of 200 mL CSF (Table XVII). In FIG. 24, NaOH uptake is plotted against tensile index. Again, the data are variable, but it is clear that despite this at a given freeness, increasing chemical uptake results in an increase in tensile strength (Pearson coefficient 0.700, p=0.022). This finding is in good agreement with previous work by Johal et al. and Jackson et al. who found that the tensile indices of aspen CTMP pulps increase with increasing chemical uptake. Intraclonal variation is again the largest component of the variability seen in the tear index data at a given freeness of 200 mL CSF (Table XVII).

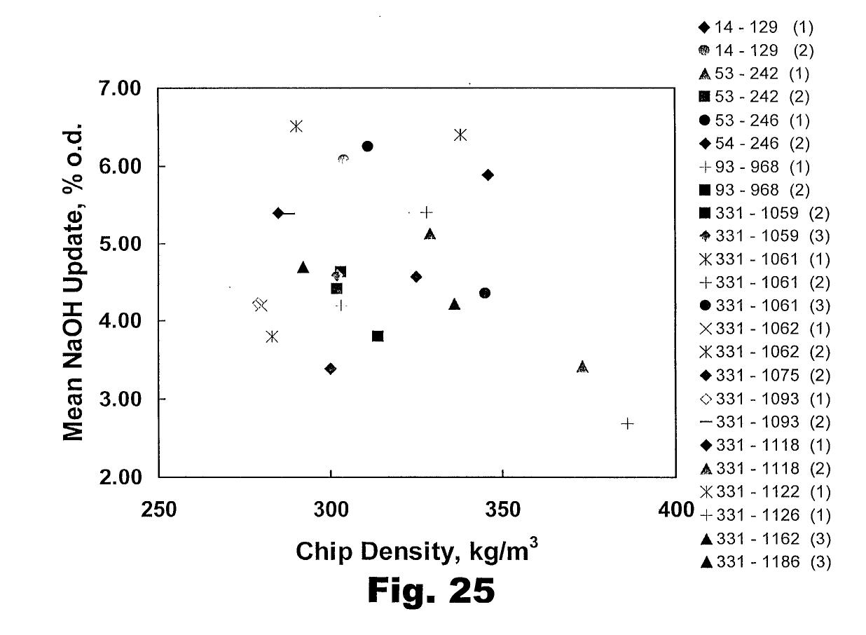

As anticipated, sheet density increases with decreasing freeness and increasing specific refining energy (Table XVII). The extent of the intra- and interclonal variability seen at 200 mL freeness, from 361 kg/M³ to 459 kg/M³, is of the same order as that previously noted for aspen clones and is shown in Table XVII. Whilst some clones (e.g. parent 93-968) produce sheets with similar density properties, others (e.g. parent 14-129) exhibit wide intraclonal variability. The role of alkali uptake at 200 mL freeness in the consolidation of sheet density of hybrid poplar clone APRMP pulps is shown in FIG. 25. The significant positive relationship seen (Pearson coefficient 0.616, p=0.001) indicates the importance of good chemical impregnation to soften fiber cell walls and improve sheet consolidation.

Surface and Optical Properties

As expected, scattering coefficient consistently increased with decreasing freeness and increasing sheet density (Table XVII). Significant positive correlations were observed between SRE and optical properties scattering coefficient (Pearson coefficient 0.779, p=0.000) and printing opacity (Pearson coefficient 0.738, p=0.003).

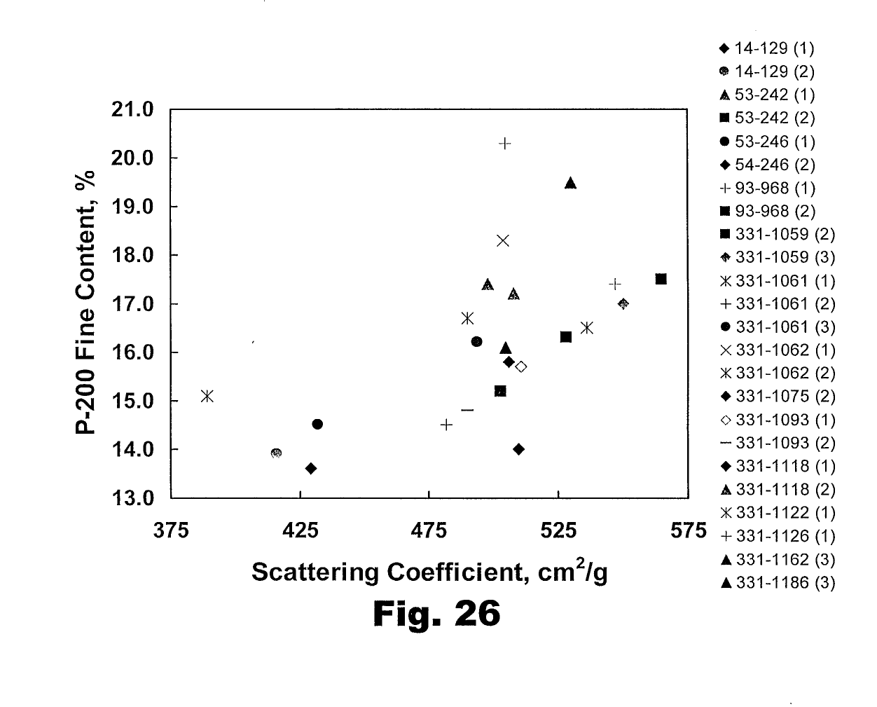

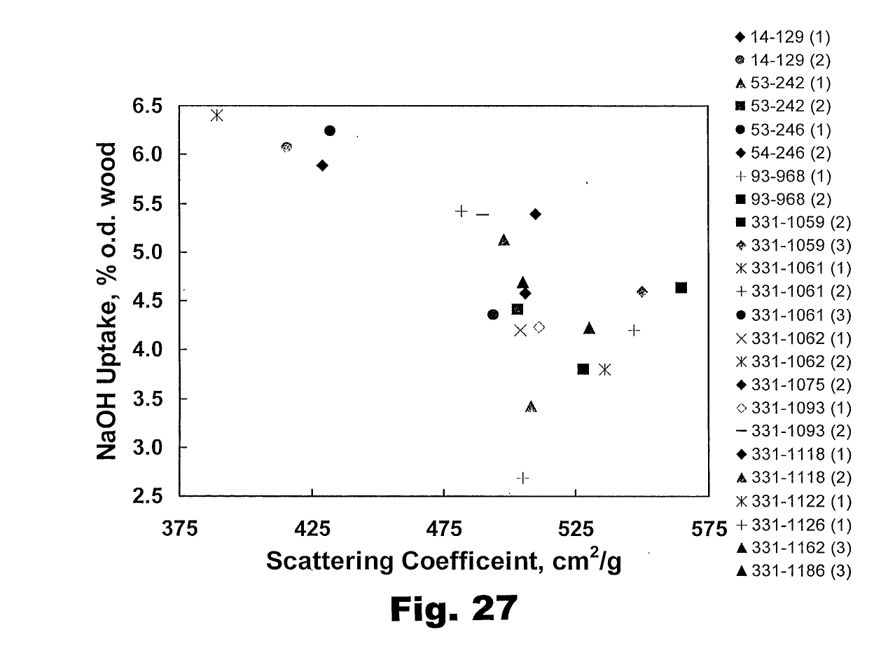

In FIG. 26, the fines content (P-200) is shown as a function of scattering coefficient. The significant positive relationship (Pearson coefficient 0.637, p=0.001) confirms previous observations for aspen in that those clones with the highest fines content also exhibit high scattering coefficients and high opacity values. The negative effect of chip alkali uptake—on light scattering development is indicated in FIG. 27 (Pearson coefficient −0.713, p=0.000). The most probable explanation for this negative effect is that increased alkali uptake makes the fiber separation at the middle lamella easier and thus producing fewer fines. Secondly, the higher alkali uptake makes the fibers more flexible and hydrophilic thus resulting in more fiber bonding and reduced light scattering.

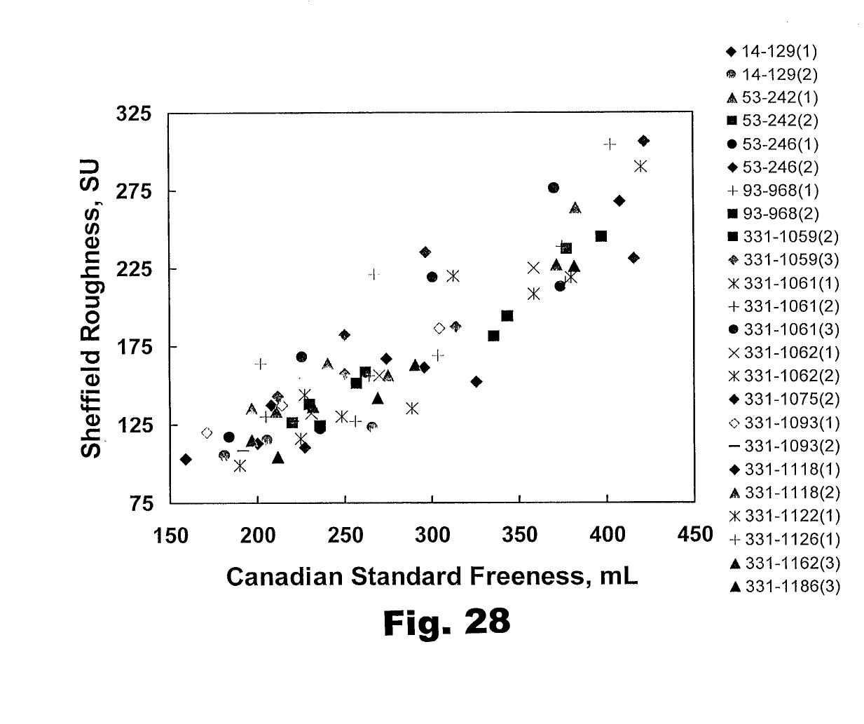

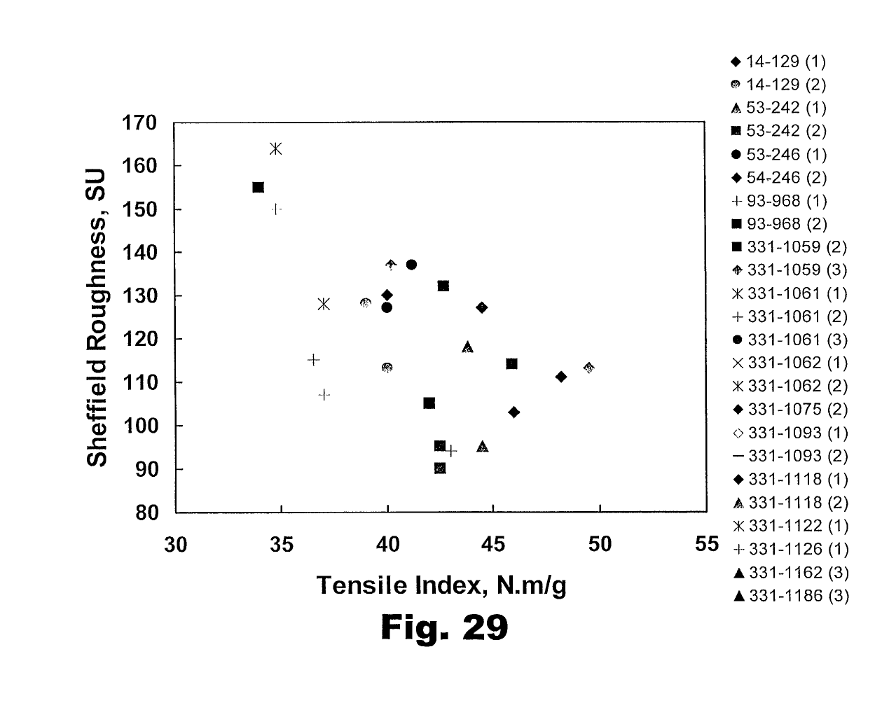

Sheffield roughness increased with increasing freeness (FIG. 28). The plot of Sheffield roughness vs. tensile strength (FIG. 29) indicates that at high tensile index, most clones exhibit excellent sheet surface properties. The significant negative relationship seen (Pearson coefficient −0.602, p=0.002) does not alter the fact that, within this hybrid population, a wide variety of pulp strengths can be had whilst maintaining a constant smoothness level (see Table XX).

TABLE XX
Interclonal variability of strength properties for
given formation properties

| Clone | Tensile index (N · m/g) | Sheffield Smoothness (SU) |
|---|---|---|
| 331-1118 (1) | 49.5 | 113 |
| 331-1162 (3) | 36.5 | 115 |

The brightness of the APRMP pulps from different clones under significantly variable H₂O₂ uptake was surprisingly similar. A tight range of brightness values was obtained from the hybrid poplar pulps, from 74-79%. This compares very well with previous brightness results for aspen clones which showed greater variability over a lower spectrum of values, from 49-69%. The aspen values may be explained by the occurrence in natural stands of highly stained wood and by wide differences in the lignin content of the examined trees.

QTL Mapping Using Pulp Properties Phenotypic Data

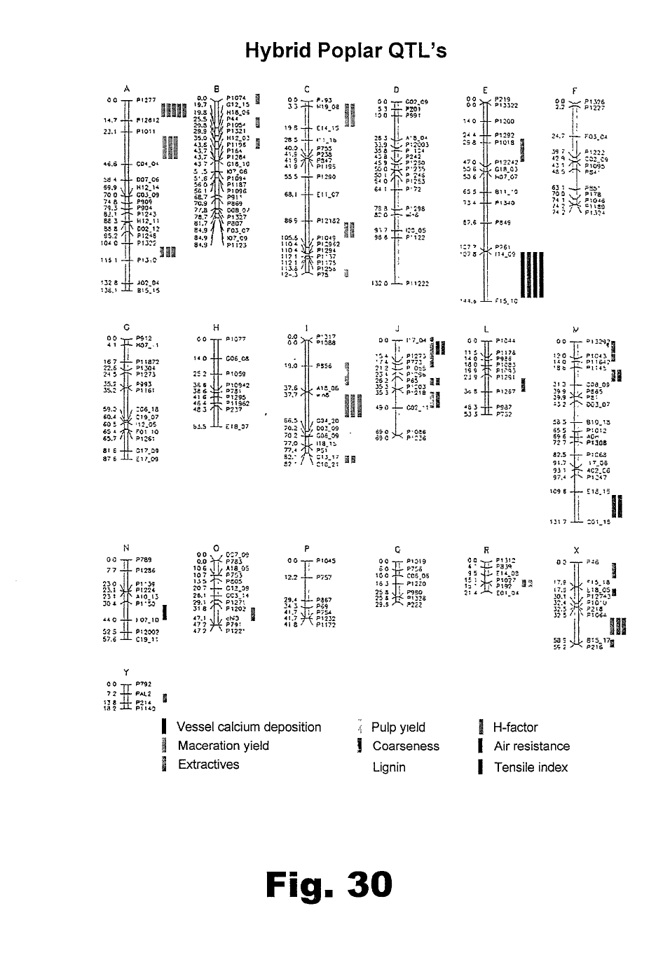

For most of the pulping parameters examined in this study, both intra- and interclonal factors were significant determinators of the population variability encountered. This, coupled with the necessarily small sample size utilized, makes the correlation of genotypic and phenotypic variability statistically challenging. Some data sets did yield significant QTL detections—for example, a putative QTL has been found for H-factor with a LOD score of 4.04 (see FIG. 30 and Table XXI). In FIG. 30, the 19 Populus linkage groups and positioned RFLP, RAPD and STS markers are shown. Positions of detected QTL which exceed the significance threshold LOD score are indicated by colour-coded vertical bars adjacent to the linkage groups. Phenotyping data colour codes are described in the legend. Importantly using the kraft pulping data, a significant QTL for tensile index (LOD score 3.48) and a less significant QTL for air resistance (LOD score 2.62) were detected in a chromosomal position coincident with that detected for fiber coarseness and microfibril angle. These results are depicted in Table XXI. These data suggest that not only does this genetic region contain genes which affect multiple related pulp parameters and is therefore worthy of further investigation, but that the coarseness values obtained from the peracetic acid maceration/FQA fiber analysis technique do indeed accurately reflect the performance of the pulp in terms of a number of important parameters. The observation strongly supports the use of this procedure as a technique for rapid assessment of tree populations for wood quality.

Most of the QTL found, however, had LOD significance scores of approximately the threshold value of 2.90 or lower, indicating a high possibility of spurious detection. QTL mapping of these data is, therefore, not presented here as the data sets are simply not extensive enough for statistical significance. These data will form part of a larger and continuing study on this population of hybrid poplars with the eventual goal of genetic mapping of specific pulping and papermaking characteristics. This is considered to be an important outcome as, as has been clearly shown by this and numerous other reports, it is often highly problematic to accurately predict pulp and papermaking properties from easily measured parameters such as fiber properties, wood density, etc. To actually determine the pulp and paper properties of a clone, it is still necessary to pilot pulp the entire stem. It is anticipated that QTL mapping of a large enough sample set of pilot pulps will enable the detection of the particular subset of genes which directly affect pulp and paper parameters and the development of rapid assessment methods for those properties of immediate industrial value. This study represents the first steps towards eventual achievement of this highly important objective.

TABLE XXI
Significant QTL detected for H factor

| Trait | Marker/Linkage | LOD Score | Phen % | Length/cM | Weight | Dom. |
|---|---|---|---|---|---|---|
| H factor | PAL2-P214/Y | 4.04 | 95.6 | 6.6 | 169.83 | −337.80 |
| Tensile index | I14_09-F15_10/E | 3.48 | 87.2 | 37.3 | 1.5378 | 9.8668 |
| Air resistance | I14_09-F15_10/E | 2.62* | 88.4 | 37.3 | 519.36 | −250.13 |
| (Gurley) | | | | | | |
| Fiber | I14_09-F15_10/E | 3.49 | 55.9 | 37.3 | 72.794 | −79.906 |
| Coarseness** | | | | | | |

*Reported due to significant location.
**Data from Table I repeated to illustrate co-localization with other reported QTL.

QTL Mapping

FIG. 30 illustrates the current status of QTL mapping using the Family 331 hybrid poplar mapping pedigree. The map shows the 19 linkage groups that are approximately equivalent to the 19 Populus chromosomes as vertical bars labelled A-Y as obtained from the University of Washington. Positions of assigned RFLP, RAPD and STS markers are indicated on each linkage group. Assigned QTL regions for each of the traits examined in the study are indicated as colour-coded bars adjacent to the linkage groups. Details on the significance of the QTL and the genetic distances they cover can be found in the appropriate tables, although it is important to note that—with the single exception of kraft pulp yield—each reported QTL exceeds the 95% statistical confidence level, as determined by the LOD threshold score of 2.9.

RAPD Analysis and Polymorphic Product Characterization

Table XVI shows the screened suite of markers associated with the QTL linked to the specific traits of interest examined in this study. Each of these RAPD/RFLP markers was used in a PCR reaction to generate a polymorphic product from the phenotypically selected F2 generation individuals indicated. Table XVI also presents the number of sequences generated from the polymorphic bands isolated. Proposed functionalities for the sequences, based on similarities to sequences already in public databases, can be found in Table XVI. The sequences are tabulated in Table XVII. The polymorphic marker bands have been fully or partially sequenced and functionality has been assigned according to similarity with previously published sequences on public databases (e.g. genbank).

By sequence homology it will now be possible to identify orthologous functional genes in trees of the genus Populus, Picea, Berula, Abies, Larix, Taxus, Ulmus, Prunus, Quercus, malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

| | | # | Product size | |
|---|---|---|---|---|
| Trait | Marker | Sequences | (bp) | Database ID |
| Maceration | I17_04 | 2 | | (AC007018) Arabidopsis thaliana |
| yield | | | | chromosome; |
| | | | | (AP002820) putative transposable |
| | | | | element Tip 100 protein RICE |
| Maceration | G02_11 | 5 | 1138, 990, | (AC006136) putative retroelement |
| yield | | | 1032, 976, 986 | pol polyprotein [Arabidopsis] |
| | | | | (AC009400) hypothetical protein |
| | | | | [Arabidopsis thaliana; |
| | | | | >gi\|13241678\|gb\|AAK16420.1\| |
| | | | | (AF320086) RIRE gag/pol protein |
| | | | | [Zea mays]; unknown; AC020580) |
| | | | | hypothetical protein, 3'partial |
| Yield/H | E01_04 | 3 | 347, 334, 356 | (AC002332) hypothetical protein |
| factor | | | | [Arabidopsis thaliana]; AC007357) |
| | | | | F3F19.15 [Arabidopsis thaliana]; |
| | | | | (AB024037) |
| | | | | emb\|CAB77928.1˜gene_id: MSK1 |
| | | | | 0.2˜similar to unknown |
| Yield/H- | P1027 | 3 | 539, 589, 593 | hypothetical protein, At; putative |
| factor | | | | retroelement; At EST ATTS1136, |
| | | | | putative disease resistance gene. |
| Lignin | P757 | 2 | 281, 199 | Arabidopsis retrotransposon-like |
| | | | | protein, Z97342. |
| Coarseness/ | I14_09 | 3 | 545, 545, 869 | unknown; |
| tensile | | | | low hits: cotton fad aj244890; |
| index/air | | | | poplar agamous (64% in 197 nt); |
| resistance | | | | copia-like polyprotein [Arabidopsis |
| | | | | thaliana] |
| | F15_10 | 2 | 950, 980 | unknown Arabidopsis gene; |
| | | | | Many proline-rich proteins (#1 = |
| | | | | cicer arietinium), +3 frame |
| Extractives | B15 | 2 | 1756, 1693 | endo-1,4-betaglucanase, |
| | | | | fibronectin repeat signature |
| | H19_08 | 1 | 810 | transformer-SR ribonucleoprotein |
| | G13_17 | 2 | 1400, 1628 | several dnaJ-like protein |
| | | | | [Arabidopsis thaliana]; |
| | | | | gi\|1491720\|emb\|CAA67813.1\| |
| | | | | (X99451) extensin-like protein |
| | | | | Dif10 [Lycopersicon esculentum |
| | G12_15 | 1 | 677 | 1 = unknown At protein, |
| | | | | 2 = hypothetical Ca-binding |
| | | | | protein from At |
| | C04_04 | 1 | 357 | genomic DNA T7N9.15 |
| | | | | [Arabidopsis thaliana] |
| | P1054 | 1 | 787 | Cicer arietinum mRNA for glucan- |
| | | | | endo-1,3-beta-glucosidase |
| | P1018 | 1 | 522 | AC007197 Arabidopsis thaliana |
| | | | | chromosome |
| | H12 | 3 | 332, 386, 350 | hypothetical protein (COP1 |
| | | | | regulatory), endoglucanase, |
| | | | | 3-oxo-5-alpha-steroid-4- |
| | | | | dehydrogenase. |
| Calcium | H07_10 | 3 | 977, 978, 754 | (AC003970) Similar to Glucose-6- |
| deposition | | | | phosphate dehydrogenases, At; |
| | | | | AC006267) putative polyprotein |
| | | | | [Arabidopsis thaliana]; |
| | | | | (AC006267) putative polyprotein |
| | | | | [Arabidopsis thaliana] |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method of identifying tree lineage capable of expressing desired biological and/or biochemical phenotypes comprising the steps of:
a) obtaining a nucleic acid sample from the trees of pure species and/or hybrids thereof;
b) obtaining either a restriction pattern (RFLP) or PCR-fingerprint by subjecting said nucleic acid of step (a) to at least one restriction enzyme and/or standard PCR conditions with at least one specific primer;
c) correlating said PCR-fingerprint or restriction pattern of step (b) to at least one selected biological and/or biochemical phenotype of said tree wherein said phenotype is associated with a genetic locus identified by and/or associated with said PCR fingerprint or restriction pattern.

2. The method according to claim 1, wherein said PCR-fingerprint is selected from the group consisting of RAPD, AFLP, CAP and SCAR.

3. The method according to claim 1, wherein said correlating of step (c) further comprises the sequencing of polymorphic DNA products associated with the genetic locus associated with the said phenotype.

4. The method according to claim 1, wherein DNA sequences represent candidate genes or are highly linked to candidate genes for use as DNA markers as in step (c).

5. The method according to claim 4, wherein said DNA sequences are physically and/or genetically linked to candidate genes.

6. The method according to claim 1, wherein said tree of pure species and/or hybrid thereof is naturally or artificially produced.

7. The method according to claim 1, wherein said sample of step (a) is obtained from a leaf, cambium, root, bud, stem, cork, phloem, flower or xylem.

8. The method according to claim 1, wherein said tree is of the genus selected from the group consisting of: Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

9. A method of identifying a genetic marker associated with a genetic locus conferring at least one enhanced property selected from the group consisting of fiber length, fiber coarseness, DBII (diameter at breast height), microfibril angle, density, pulp strength, pulp yield, lignin content, pitch propensity and calcium accumulation in a family of trees, which comprises the steps of:
a) obtaining a sexually mature parent tree exhibiting enhanced properties;
b) obtaining a plurality of progeny trees of said parent tree by performing self or cross-pollination;
c) assessing multiple progeny trees for each of a plurality of genetic markers;
d) identifying genetic markers segregating in an essentially Mendelian ratio and showing linkage with at least some other of said plurality of genetic markers;
e) measuring at least one of said properties in multiple progeny trees; and
f) correlating the presence of enhanced property with a least one marker identified in step d) as segregating in an essentially Mendelian ratio and showing linkage with at least some of said other markers, the correlation of the presence of enhanced properties with a marker indicating that said marker is associated with a genetic locus conferring enhanced; wherein said family of trees comprises a parent tree and its progeny.

10. The method of claim 9, further comprising constructing a genetic linkage map of said parent tree using said plurality of genetic markers.

11. The method of claim 10, wherein said genetic linkage map is a QTL map.

12. The method of claim 9, wherein said genetic marker loci are restriction fragment length polymorphism (RFLPs) or PCR-fingerprint.

13. The method of claim 12, wherein said PCR-fingerprint is selected from the group consisting of RAPD, AFLP, CAP and SCAR.

14. The method of claim 12, wherein said restriction fragment length polymorphism (RFLPs) or PCR-fingerprint are correlated with a locus or with a quantitative traits loci (QTLs).

15. The method of claim 14, wherein said PCR-fingerprint is selected from the group consisting of RAPD, AFLP, CAP and SCAR.

16. The method of claim 9, wherein said parent tree is the seed parent tree to each of said progeny trees, root, leaf or cambium tissue from said progeny trees is assessed for the presence or absence of genetic markers in step c).

17. The method of claim 9, wherein said parent tree is of the genus selected from the group consisting of Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

18. The method of claim 9, wherein said parent tree is a species of Populus trichocarpa, Populus deltoides, Populus tremuloides or a hybrid thereof.

19. A method of producing a plurality of clonal trees that have at least one enhanced property selected from the group consisting of fiber length, fiber coarseness, DBII (diameter at breast height), microfibril angle, density, pulp strength, pulp yield, lignin content, pitch propensity and calcium accumulation, which comprises the steps of:
a) obtaining a sexually mature parent tree exhibiting enhanced property relative to a value characteristic of the average of the genus;
b) obtaining a plurality of progeny trees of said parent tree by performing self or cross-pollination;
c) assessing multiple progeny tress for each of a plurality of genetic markers; d) identifying genetic markers segregating in an essentially Mendelian ratio and showing linkage with at least some other of said plurality of genetic markers;
e) measuring at least one of said properties in multiple progeny trees;
f) correlating the presence of enhanced property with a least one marker identified in step d) as segregating in an essentially Mendelian ratio and showing linkage with at least some of said other markers;
g) selecting a progeny tree containing a marker identified in step f) as associated with a genetic locus conferring enhanced property; and
h) vegetatively propagating said progeny tree selected in step g) to produce a plurality of clonal trees, essentially all of said clonal trees exhibiting enhanced fiber length.

20. The method of claim 19, further comprising constructing a genetic linkage map of said parent tree using said plurality of genetic markers.

21. The method of claim 20, wherein said genetic linkage map is a QTL map.

22. The method of claim 19, wherein said genetic marker loci are restriction fragment length polymorphism (RFLPs) or PCR-fingerprint.

23. The method of claim 22, wherein said PCR-fingerprint is selected from the group consisting of RAPD, AFLP, CAP and SCAR.

24. The method of claim 19, wherein said restriction fragment length polymorphism (RFLPs) or PCR-fingerpring are correlated with a single locus or with a quantitative traits loci (QTLs).

25. The method of claim 24, wherein said PCR-fingerprint is selected from the group consisting of RAPD, AFLP, CAP and SCAR.

26. The method of claim 19, wherein said parent tree is the seed parent tree to each of said progeny trees, root and leaf or cambium tissue from said progeny trees is assessed for the presence or absence of genetic markers in step c).

27. The method of claim 19, wherein said parent tree is of the genus selected from the group consisting of Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

28. The method of claim 19, wherein said parent tree is a species of Populus trichocarpa, Populus deltoides, Populus tremuloides or a hybrid thereof.

29. A stand of clonal enhanced property trees produced by the method of claim 19, the genome of said trees containing the same genetic marker associated with said enhanced property relative to a value characteristic of the average of the genus.

30. A method of producing a family of trees wherein at least about half exhibit at least of enhanced property selected from the group consisting of fiber length, fiber coarseness, DBII (diameter at breast height), microfibril angle, density, pulp strength, pulp yield, lignin content, pitch propensity and calcium accumulation, which comprises the steps of:
a) obtaining a sexually mature parent tree exhibiting enhanced property relative to a value characteristic of the average of the genus;
b) obtaining a plurality of progeny trees of said parent tree by performing self or cross-pollination;
c) assessing multiple progeny tress for each of a plurality of genetic markers;
d) identifying genetic markers segregating in an essentially Mendelian ratio and showing linkage with at least some other of said plurality of genetic markers;
e) measuring at least one of said properties in multiple progeny trees;
f) correlating the presence of enhanced fiber length with a least one marker identified in step d) as segregating in an essentially Mendelian ratio and showing linkage with at least some of said other markers;
g) selecting a progeny tree containing a marker identified in step f) as associated with a genetic locus conferring enhanced property; and
h) sexually propagating said progeny tree selected in step g) to produce a family of trees, at least about half of said family of trees containing a genetic locus conferring enhanced property and said family of trees exhibiting enhanced property.

31. The method of claim 30, further comprising constructing a genetic linkage map of said parent tree using said plurality of genetic markers.

32. The method of claim 31, wherein said genetic linkage map is a QTL map.

33. The method of claim 30, wherein said genetic marker loci are restriction fragment length polymorphism (RFLPs) or PCR-fingerprint.

34. The method of claim 33, wherein said PCR-fingerprint is selected from the group consisting of RAPD, AFLP, CAP and SCAR.

35. The method of claim 33, wherein said restriction fragment length polymorphism (RFLPs) or PCR-fingerprint are correlated with a locus or with a quantitative traits loci (QTLs).

36. The method of claim 35, wherein said PCR-fingerprint is selected from the group consisting of RAPD, AFLP, CAP and SCAR.

37. The method of claim 30, wherein said parent tree is the seed parent tree to each of said progeny trees, root, leaf or cambium tissue from said progeny trees is assessed for the presence or absence of genetic markers in step c).

38. The method of claim 30, wherein said parent tree is of the genus selected from the group consisting of Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

39. The method of claim 30, wherein said parent tree is a species of Populus trichocarpa, Populus deltoides, Populus tremuloides or a hybrid thereof.

40. A genetic map of QTLs of trees associated with enhanced properties as set forth in FIG. 30.

41. The genetic map of claim 40, wherein said enhanced properties are selected from the group consisting of fiber length, fiber coarseness, DBII (diameter at breast height), microfibril angle, density, pulp strength, pulp yield, lignin content, pitch propensity and calcium accumulation.

42. A genetic marker of fiber length of trees, which comprises a 800 bp amplification product, wherein presence of said product in an amplified DNA sample from said trees is indicative of a short fiber length <0.92 mm and absence of said product is indicative of long fiber length >0.92 mm.
